(19) United States
(12) Patent Application Publication
(10) Pub. No.: US 2012/0190701 A1
(43) Pub. Date: Jul. 26, 2012

(54) RENIN INHIBITORS

(75) Inventors: Austin Chih-Yu Chen, San Marcos, CA (US); Sebastien Laliberte, Saint-Lazare (CA); Guillaume Larouche, St-Jean sur Richelieu (CA); Yongxin Han, Montreal (CA); Daniel McKay, Milton, MA (US)

(73) Assignee: Merck Sharp & Dohme Corp., Rahway, NJ (US)

(21) Appl. No.: 13/390,465

(22) PCT Filed: Aug. 17, 2010
(86) PCT No.: PCT/CA2010/001281

§ 371 Date: Feb. 14, 2012

Related U.S. Application Data
(60) Provisional application No. 61/234,827, filed on Aug. 18, 2009.

(51) Int. Cl. A61K 31/438; C07D 491/20; A61K 31/4709; A61K 31/4725; A61P 9/12; A61P 25/22; A61P 9/00; A61P 9/10; A61P 13/12; A61P 25/00; A61P 27/02; A61P 27/06; C07D 491/107; A61P 9/04

(52) U.S. Cl. 514/278; 546/17

(57) ABSTRACT

Renin inhibitors, which are spirocyclic piperidine amides, of structural formula (I) and pharmaceutical compositions thereof useful in the treatment of cardiovascular diseases and renal insufficiency. wherein n, for each instance in which it occurs, is independently 0, 1, or 2; R¹ is hydrogen, C₁₋₆-alkyl or C₃₋₆-cycloalkyl, wherein said C₁₋₆-alkyl or C₃₋₆-cycloalkyl group can be independently substituted with 1-3 halogens; A is (i) a five- or six-membered saturated or unsaturated heterocyclic or carbocyclic monocyclic ring or (ii) a five- or six-membered saturated or unsaturated heterocyclic or carbocyclic ring which is fused to another five- or six-membered saturated or unsaturated heterocyclic or carbocyclic ring, V is a bond or —(C═O)—, —CH(OH)—, —CH₂—or ═CH—; U is a bond or —CH2-, or for the case when V is ═CH—, U is —CH═; X is ═CH—, ═CF—, ═C(OR³)—, or —C═O—; and Y is ═CH—, ═CF—, ═N—, or for the case when X is —C═O—, Y is —N(R³)—.

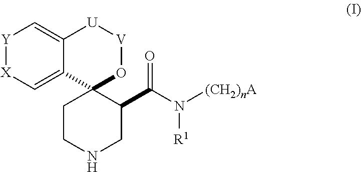

### JOINT RESEARCH AGREEMENT

The claimed invention was made pursuant to activities within the scope of a joint research agreement between Merck & Co., Inc. and Actelion Pharmaceuticals Ltd. executed on Dec. 4, 2003.

### FIELD OF THE INVENTION

The invention relates to novel renin inhibitors of general formula (I). The invention also concerns related aspects including pharmaceutical compositions containing one or more compounds of formula (I) and their use as renin inhibitors, particularly for the treatment of cardiovascular events and renal insufficiency.

### BACKGROUND OF THE INVENTION

In the renin-angiotensin system (RAS), biologically active angiotensin II (Ang II) is generated via a two-step mechanism. The highly specific renin enzyme initially cleaves angiotensinogen to angiotensin I (Ang I), which is then further processed to Ang II by the less specific angiotensin-converting enzyme (ACE). Ang II is currently known to act on four receptor subtypes, AT₁₋₄. AT₁ seems to transmit most of the known functions of Ang II, i.e., vasoconstriction, increased cardiac contractility, renal tubular sodium reabsorption, vascular and cardiac hypertrophy, etc. (see, e.g., Lévy, B. I., Circulation, 2004, 109, 8). AT₂₋₄ are less well-characterized; AT₂ may antagonize the effects of AT₁ (see, e.g., Porrello, E. R. et al., Frontiers in Bioscience, 2009, 14, 958).

Modulation of the RAS thus represents a major target for the treatment of cardiovascular diseases. ACE inhibitors and angiotensin receptor blockers (ARBs) have been used to treat hypertension. In addition, ACE inhibitors are in clinical use for renal protection (Kshirsagar, K. V. et al., American Journal of Kidney Diseases, 2000, 35, 695), the prevention of congestive heart failure (Konstam M. A. et al., Circulation, 1992, 6, 431) and treatment after myocardial infarction (Pfeffer, M. A. et al., N. Engl. J. Med., 1992, 327, 669).

Renin inhibitors present an attractive therapeutic approach due to the specificity of renin (Kleinert H. D., Cardiovasc. Drugs, 1995, 9, 645; McInnes, G. T., J. Human Hypertension, 2007, 21, 766). The only substrate known for renin is angiotensinogen, which can only be processed (under physiological conditions) by renin. By contrast, ACE cleaves bradykinin in addition to Ang I, and Ang I can also be cleaved by chymase, a serine protease (Husain A., J. Hypertens., 1993, 11, 1155). In some patients administration of ACE inhibitors leads to bradykinin accumulation, causing cough and potentially life-threatening angioneurotic edema (Israili Z. H. et al., Annals of Internal Medicine, 1992, 117, 234). Importantly, because chymase is not inhibited by ACE inhibitors, the formation of Ang II can still occur in patients treated with ACE inhibitors.

Blockade of the AT_{I} receptor by ARBs such as losartan results in increased levels of circulating Ang II and it has been suggested that AT₂ receptor stimulation may be harmful in the longer term (see, e.g., Reudelhuber, T. L., Hypertension. 2005, 46, 1261).

As renin inhibitors would be expected to demonstrate a different pharmaceutical profile than ACE inhibitors and ARBs with regard to efficacy in blocking the RAS, they may represent an alternative to some of the more harmful aspects of these agents.

The compounds of the present invention inhibit renin and represent a novel structural class of renin inhibitors. These non-peptidic compounds are orally active and of low molecular weight. They are useful for any of those clinical indications in which renin inhibition may be desirable.

### SUMMARY OF THE INVENTION

The present invention is directed to compounds of structural formula (I)

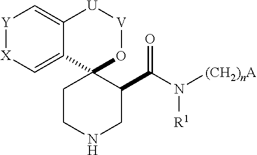

and pharmaceutically acceptable salts thereof, wherein:

n, for each instance in which it occurs, is independently 0, 1, or 2;

R¹ is hydrogen, C₁₋₆-alkyl or C₃₋₆-cycloalkyl, wherein said C₁₋₆-alkyl or C₃₋₆-cycloalkyl group can be independently substituted with 1-3 halogens;

A is (i) a five- or six-membered saturated or unsaturated heterocyclic or carbocyclic monocyclic ring (“monocyclic ring”) or (ii) a five- or six-membered saturated or unsaturated heterocyclic or carbocyclic ring which is fused to another five- or six-membered saturated or unsaturated heterocyclic or carbocyclic ring (“fused ring”),

wherein the heterocyclic ring(s) of (i) or (ii) contains from 1-3 heteroatoms, independently selected from N, O and S, wherein each N is optionally in the form of an oxide and each S is optionally in the form of an oxide selected from the group consisting of S(═O) and S(═O)₂,

wherein the heterocyclic or carbocyclic ring(s) of (i) or (ii) is optionally substituted by 1-4 radicals independently selected from the group consisting of:
(1) halogen,
(2) cyano,
(3) C₁₋₆ alkyl,
(4) C₁₋₆ alkanoyl,
(5) C₁₋₆ alkoxy,
(6) C₂₋₆ alkenyl,
(7) C₃₋₆ cycloalkyl,
(8) carboxy-C₁₋₆ alkyl,
(9) carboxy-C₃₋₆ cycloalkyl,

wherein substituents (3)-(9) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or W, wherein W is morpholine, oxomorpholine, pyrrolidine, succinimide, acylmorpholine, or thiomorpholine 1,1-dioxide;

R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group can be independently substituted with 1-3 halogens;

R³ is hydrogen, C₁₋₄ alkyl or C₃₋₆ cycloalkyl, wherein said C₁₋₄ alkyl or C₃₋₆ cycloalkyl group can be independently substituted with 1-3 halogens;

V is a bond or —(C═O)—, —CH(OH)—, —CH₂— or ═CH—;

U is a bond or —CH₂—, or for the case when V is ═CH—, U is —CH═;

X is ═CH—, ═CF—, ═C(OR³)—, or —(C═O)—; and

Y is ═CH—, ═CF—, ═N—, or for the case when X is —(C═O)—, Y is —N(R³)—.

The present invention further relates to processes for preparation of the compounds as well as pharmaceutical compositions containing one or more of said compounds in free form or in pharmaceutically acceptable salt form, together with one or more customary pharmaceutical excipient(s), as well as methods for inhibition of renin activity and of treatment for conditions in which renin inhibition may have a therapeutic effect. Such conditions include hypertension, congestive heart failure, cardiac hypertrophy, cardiac fibrosis, postinfarction cardiomyopathy, nephropathy, vasculopathy, neuropathy, restenosis following angioplasty, raised intra-ocular pressure, glaucoma, abnormal vascular growth, hyperaldosteronism, and anxiety states.

The present invention also relates to methods of inhibiting renin activity, wherein said method comprises the step of administering a compound according to formula (I) in an amount sufficient to provide an effective amount for renin inhibition in an organism.

### DETAILED DESCRIPTION OF THE INVENTION

The present invention is directed to compounds of Formula (I):

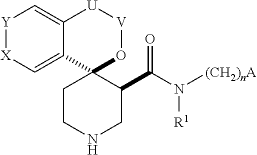

and pharmaceutically acceptable salts thereof, wherein:

n, for each instance in which it occurs, is independently 0, 1, or 2;

R¹ is hydrogen, C₁₋₆-alkyl or C₃₋₆-cycloalkyl, wherein said C₁₋₆-alkyl or C₃₋₆-cycloalkyl group can be independently substituted with 1-3 halogens;

A is (i) a five- or six-membered saturated or unsaturated heterocyclic or carbocyclic monocyclic ring (“monocyclic ring”) or (ii) a five- or six-membered saturated or unsaturated heterocyclic or carbocyclic ring which is fused to another five- or six-membered saturated or unsaturated heterocyclic or carbocyclic ring (“fused ring”),

wherein the heterocyclic or carbocyclic ring(s) of (i) or (ii) is optionally substituted by 1-4 radicals independently selected from the group consisting of:

(1) halogen,

(2) cyano,

(3) C₁₋₆ alkyl,

(4) C₁₋₆ alkanoyl,

(5) C₁₋₆ alkoxy,

(6) C₂₋₆ alkenyl,

(7) C₃₋₆ cycloalkyl,

(8) carboxy-C₁₋₆ alkyl,

(9) carboxy-C₃₋₆ cycloalkyl,

wherein substituents (3)-(9) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or W, wherein W is morpholine, oxomorpholine, pyrrolidine, succinimide, acylmorpholine, or thiomorpholine 1,1-dioxide;

R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group can be independently substituted with 1-3 halogens;

R³ is hydrogen, C₁₋₄ alkyl or C₃₋₆ cycloalkyl, wherein said C₁₋₄ alkyl or C₃₋₆ cycloalkyl group can be independently substituted with 1-3 halogens;

V is a bond or —(C═O)—, —CH(OH)—, —CH₂— or ═CH—;

U is a bond or —CH₂—, or for the case when V is ═CH—, U is —CH═;

X is ═CH—, ═CF—, ═C(OR³)—, or —(C═O)—; and

Y is ═CH—, ═CF—, ═N—, or for the case when X is —(C═O)—, Y is —N(R³)—.

In a preferred embodiment, a compound of formula (Ia) is provided:

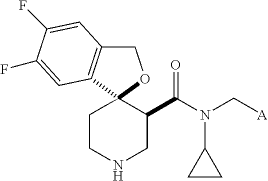

wherein A is (i) a five- or six-membered saturated or unsaturated heterocyclic or carbocyclic monocyclic ring (“monocyclic ring”) or (ii) a five- or six-membered saturated or unsaturated heterocyclic or carbocyclic ring which is fused to another five- or six-membered saturated or unsaturated heterocyclic or carbocyclic ring (“fused ring”),

wherein the heterocyclic ring(s) of (i) or (ii) contains from 1-3 heteroatoms, independently selected from N, O and S, wherein each N is optionally in the form of an oxide and each S is optionally in the form of an oxide selected from the group consisting of S(═O) and S(═O)₂,

wherein the heterocyclic or carbocyclic ring(s) of (i) or (ii) is optionally substituted by 1-4 radicals independently selected from the group consisting of:

(1) halogen,

(2) cyano,

(3) C₁₋₆ alkyl,

(4) C₁₋₆ alkanoyl,

(5) C₁₋₆ alkoxy,

(6) C₂₋₆ alkenyl,

(7) C₃₋₆ cycloalkyl,

(8) carboxy-C₁₋₆ alkyl,

(9) carboxy-C₃₋₆ cycloalkyl,

wherein substituents (3)-(9) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or W, wherein W is morpholine, oxomorpholine, pyrrolidine, succinimide, acylmorpholine, or thiomorpholine 1,1-dioxide;

R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group can be independently substituted with 1-3 halogens;

R³ is hydrogen, C₁₋₄ alkyl or C₃₋₆ cycloalkyl, wherein said C₁₋₄ alkyl or C₃₋₆ cycloalkyl group can be independently substituted with 1-3 halogens.

More particularly, A is selected from
(1) substituted or unsubstituted aryl, selected from benzyl or naphthyl; and
(2) substituted or unsubstituted heteroaryl, selected from quinolinyl; isoquinolinyl; pyridinyl; benzopyridinyl; or indolyl.

Specific examples of compounds of formula I, and pharmaceutically acceptable salts thereof, include the following:
(cis-1,3′)-N-Cyclopropyl-N-[3-(2-methoxyethoxy)-5-(3-methoxypropyl)benzyl]-3-oxo-3H-spiro[2-benzofuran-1,4′-piperidine]-3′-carboxamide (Example 1);
(cis-1,3′)-N-Cyclopropyl-3-hydroxy-N-[3-(2-methoxyethoxy)-5-(3-methoxypropyl)benzyl]-3H-spiro[2-benzofuran-1,4′-piperidine]-3′-carboxamide (Example 2);
(cis-1,3′)-N-Cyclopropyl-N-[3-(2-methoxyethoxy)-5-(3-methoxypropyl)benzyl]-3H-spiro[2-benzofuran-1,4′-piperidine]-3′-carboxamide (Example 3);
(cis-1,3′)-N-[2-Chloro-5-(2-methoxyethyl)benzyl]-N-cyclopropyl-3-oxo-3H-spiro[2-benzofuran-1,4′-piperidine]-3′-carboxamide (Example 4);
(cis-1,3′)-N-Cyclopropyl-N-[3-(2-methoxyethoxy)-5-(3-methoxypropyl)benzyl]-3,4-dihydrospiro[isochromene-1,4′-piperidine]-3′-carboxamide (Example 5);
(cis-1,3′)-N-Cyclopropyl-5,6-difluoro-N-[3-(2-methoxyethoxy)-5-(3-methoxypropyl)benzyl]-3-oxo-3H-spiro[2-benzofuran-1,4′-piperidine]-3′-carboxamide (Example 6);
(cis-1,3′)-N-[2-Chloro-5-(2-methoxyethyl)benzyl]-N-cyclopropyl-5,6-difluoro-3-oxo-3H-spiro[2-benzofuran-1,4′-piperidine]-3′-carboxamide (Example 7);
(cis-1,3′)-N-Cyclopropyl-5,6-difluoro-N-[3-(2-methoxyethoxy)-5-(3-methoxypropyl)benzyl]-3H-spiro[2-benzofuran-1,4′-piperidine]-3′-carboxamide (Example 8);
(cis-1,3′)-N-[2-Chloro-5-(2-methoxyethyl)benzyl]-N-cyclopropyl-5,6-difluoro-3H-spiro[2-benzofuran-1,4′-piperidine]-3′-carboxamide (Example 9);
(cis-1,3′)-N-Cyclopropyl-N-[3-(2-methoxyethoxy)-5-(3-methoxypropyl)benzyl]spiro[isochromene-1,4′-piperidine]-3′-carboxamide (Example 10)
(cis-1,3′)-N-[2-Chloro-5-(2-methoxyethyl)benzyl]-N-cyclopropylspiro[isochromene-1,4′-piperidine]-3′-carboxamide (Example 11);
(cis-1,3′)-N-Cyclopropyl-N-[3-(2-methoxyethoxy)-5-(3-methoxypropyl)benzyl]-3-oxo-3,4-dihydrospiro[isochromene-1,4′-piperidine]-3′-carboxamide (Example 12);
(cis-1,3′)-N-[2-Chloro-5-(2-methoxyethyl)benzyl]-N-cyclopropyl-5-methyl-6-oxo-5,6-dihydro-3H-spiro[furo[3,4-c]pyridine-1,4′-piperidine]-3′-carboxamide (Example 13);
(cis-1,3′)-N-[2-Chloro-5-(2-methoxyethyl)benzyl]-N-cyclopropyl-6-methoxy-3H-spiro[furo[3,4-c]pyridine-1,4′-piperidine]-3′-carboxamide (Example 14);
(cis-1,3′)-N-Benzyl-N-cyclopropyl-5,6-difluoro-3H-spiro[2-benzofuran-1,4′-piperidine]-3′-carboxamide (Example 15);
(cis-1,3′)-N-Cyclopropyl-N-(2,3-dichlorobenzyl)-5,6-difluoro-3H-spiro[2-benzofuran-1,4′-piperidine]-3′-carboxamide (Example 16);
(cis-1,3′)-N-Cyclopropyl-N-(2,3-dimethylbenzyl)-5,6-difluoro-3H-spiro[2-benzofuran-1,4′-piperidine]-3′-carboxamide (Example 17);
(cis-1,3′)-N-Cyclopropyl-5,6-difluoro-N-(4-quinolinylmethyl)-3H-spiro[2-benzofuran-1,4′-piperidine]-3′-carboxamide (Example 18);
(cis-1,3′)-N-[3-Bromo-5-(3-hydroxypropyl)-4-methylbenzyl]-N-cyclopropyl-5,6-difluoro-3H-spiro[2-benzofuran-1,4′-piperidine]-3′-carboxamide (Example 19);
(cis-1,3′)-N-[3-Bromo-5-(3-methoxypropyl)-4-methylbenzyl]-N-cyclopropyl-5,6-difluoro-3H-spiro[2-benzofuran-1,4′-piperidine]-3′-carboxamide (Example 20);
(cis-1,3′)-N-Cyclopropyl-5,6-difluoro-N-(1-naphthylmethyl)-3H-spiro[2-benzofuran-1,4′-piperidine]-3′-carboxamide (Example 21);
(cis-1,3′)-N-Cyclopropyl-N-(3,4-dichlorobenzyl)-5,6-difluoro-3H-spiro[2-benzofuran-1,4′-piperidine]-3′-carboxamide (Example 22);
(cis-1,3′)-N-(5-Bromo-2-chlorobenzyl)-N-cyclopropyl-5,6-difluoro-3H-spiro[2-benzofuran-1,4′-piperidine]-3′-carboxamide (Example 23);
(cis-1,3′)-N-Cyclopropyl-N-[3-(difluoromethoxy)benzyl]-5,6-difluoro-3H-spiro[2-benzofuran-1,4′-piperidine]-3′-carboxamide (Example 24);
(cis-1,3′)-N-Cyclopropyl-5,6-difluoro-N-[(2-methyl-1-napthyl)methyl]-3H-spiro[2-benzofuran-1,4′-piperidine]-3′-carboxamide (Example 25);
(cis-1,3′)-N-Cyclopropyl-5,6-difluoro-N-(8-quinolinylmethyl)-3H-spiro[2-benzofuran-1,4′-piperidine]-3′-carboxamide (Example 26);
(cis-1,3′)-N-Cyclopropyl-N-(2,3-dimethoxybenzyl)-5,6-difluoro-3H-spiro[2-benzofuran-1,4′-piperidine]-3′-carboxamide (Example 27);
(cis-1,3′)-N-Cyclopropyl-5,6-difluoro-N-[(2-methyl-3-pyridinyl)methyl]-3H-spiro[2-benzofuran-1,4′-piperidine]-3′-carboxamide (Example 28);
(cis-1,3′)-N-Cyclopropyl-5,6-difluoro-N-[2-methoxy-5-(2-methoxyethyl)benzyl]-3H-spiro[2-benzofuran-1,4′-piperidine]-3′-carboxamide (Example 29);
(cis-1,3′)-N-Cyclopropyl-5,6-difluoro-N-(1-isoquinolinylmethyl)-3H-spiro[2-benzofuran-1,4′-piperidine]-3′-carboxamide (Example 30);
(cis-1,3′)-N-[(3-Bromo-1-naphthyl)methyl]-N-cyclopropyl-5,6-difluoro-3H-spiro[2-benzofuran-1,4′-piperidine]-3′-carboxamide (Example 31);
(cis-1,3′)-N-Cyclopropyl-5,6-difluoro-N-{[3-(3-methoxypropyl)-1-naphthyl]methyl}-3H-spiro[2-benzofuran-1,4′-piperidine]-3′-carboxamide (Example 32);
(cis-1,3′)-N-Cyclopropyl-5,6-difluoro-N-[(6-methyl-2-pyridinyl)methyl]-3H-spiro[2-benzofuran-1,4′-piperidine]-3′-carboxamide (Example 33);
(cis-1,3′)-N-Cyclopropyl-5,6-difluoro-N-[(3-methyl-2-pyridinyl)methyl]-3H-spiro[2-benzofuran-1,4′-piperidine]-3′-carboxamide (Example 34);
(cis-1,3′)-N-(2-Chlorobenzyl)-N-cyclopropyl-5,6-difluoro-3H-spiro[2-benzofuran-1,4′-piperidine]-3′-carboxamide (Example 35);
(cis-1,3′)-N-(2-Bromobenzyl)-N-cyclopropyl-5,6-difluoro-3H-spiro[2-benzofuran-1,4′-piperidine]-3′-carboxamide (Example 36);
(cis-1,3′)-N-Cyclopropyl-5,6-difluoro-N-[2-(trifluoromethyl)benzyl]-3H-spiro[2-benzofuran-1,4′-piperidine]-3′-carboxamide (Example 37);
(cis-1,3′)-N-Cyclopropyl-5,6-difluoro-N-[2-methyl-3-(trifluoromethyl)benzyl]-3H-spiro[2-benzofuran-1,4′-piperidine]-3′-carboxamide (Example 38);
(cis-1,3′)-N-[(4-Bromo-1-naphthyl)methyl]-N-cyclopropyl-5,6-difluoro-3H-spiro[2-benzofuran-1,4′-piperidine]-3′-carboxamide (Example 39);
(cis-1,3′)-N-[3-Chloro-5-(trifluoromethyl)benzyl]-N-cyclopropyl-5,6-difluoro-3H-spiro[2-benzofuran-1,4′-piperidine]-3′-carboxamide (Example 40);
(cis-1,3′)-N-(2-Chloro-4-fluorobenzyl)-N-cyclopropyl-5,6-difluoro-3H-spiro[2-benzofuran-1,4′-piperidine]-3′-carboxamide (Example 41);
methyl 3-chloro-4-[(cyclopropyl{[(cis-1,3′)-5,6-difluoro-3H-spiro[2-benzofuran-1,4′-piperidin]-3′-yl]carbonyl}amino)methyl]benzoate (Example 42);
(cis-1,3′)-N-[2-Chloro-3-(trifluoromethyl)benzyl]-N-cyclopropyl-5,6-difluoro-3H-spiro[2-benzofuran-1,4′-piperidine]-3′-carboxamide (Example 43);
(cis-1,3′)-N-Cyclopropyl-5,6-difluoro-N-[2-methyl-5-(trifluoromethyl)benzyl]-3H-spiro[2-benzofuran-1,4′-piperidine]-3′-carboxamide (Example 44);
(cis-1,3′)-N-Cyclopropyl-5,6-difluoro-N-[5-(2-methoxyethyl)-2,3-dimethylbenzyl]-3H-spiro[2-benzofuran-1,4′-piperidine]-3′-carboxamide (Example 45);
(cis-1,3′)-N-Cyclopropyl-5,6-difluoro-N-[5-(3-methoxypropyl)-2,3-dimethylbenzyl]-3H-spiro[2-benzofuran-1,4′-piperidine]-3′-carboxamide (Example 46);
(cis-1,3′)-N-{3-[2-(Acetylamino)ethyl]benzyl}-N-cyclopropyl-5,6-difluoro-3H-spiro[2-benzofuran-1,4′-piperidine]-3′-carboxamide (Example 47);
(cis-1,3′)-N-Cyclopropyl-5,6-difluoro-N-{[2-(3-methoxypropyl)-4-quinolinyl]methyl}-3H-spiro[2-benzofuran-1,4′-piperidine]-3′-carboxamide (Example 48);
(cis-1,3′)-N-{3-[(Acetylamino)methyl]benzyl}-N-cyclopropyl-5,6-difluoro-3H-spiro[2-benzofuran-1,4′-piperidine]-3′-carboxamide (Example 49);
(cis-1,3′)-N-[(6-Bromo-8-quinolinyl)methyl]-N-cyclopropyl-5,6-difluoro-3H-spiro[2-benzofuran-1,4′-piperidine]-3′-carboxamide (Example 50);
(cis-1,3′)-N-[(2-Bromo-3-pyridinyl)methyl]-N-cyclopropyl-5,6-difluoro-3H-spiro[2-benzofuran-1,4′-piperidine]-3′-carboxamide (Example 51);
(cis-1,3′)-N-[(6-Chloro-3-pyridinyl)methyl]-N-cyclopropyl-5,6-difluoro-3H-spiro[2-benzofuran-1,4′-piperidine]-3′-carboxamide (Example 52);
(cis-1,3′)-N-[(2-Chloro-3,5-bis(3-methoxypropyl)benzyl]-N-cyclopropyl-5,6-difluoro-3H-spiro[2-benzofuran-1,4′-piperidine]-3′-carboxamide (Example 53);
(cis-1,3′)-N-Cyclopropyl-5,6-difluoro-N-{[6-(3-methoxypropyl)-8-quinolinyl]methyl}-3H-spiro[2-benzofuran-1,4′-piperidine]-3′-carboxamide (Example 54);
(cis-1,3′)-N-[5-Bromo-2-methyl-3-(trifluoromethyl)benzyl]-N-cyclopropyl-5,6-difluoro-3H-spiro[2-benzofuran-1,4′-piperidine]-3′-carboxamide (Example 55);
(cis-1,3′)-N-cyclopropyl-5,6-difluoro-N-[(4-fluoro-1H-indol-3-yl)methyl]-3H-spiro[2-benzofuran-1,4′-piperidine]-3′-carboxamide (Example 56);
(cis-1,3′)-N-Cyclopropyl-5,6-difluoro-N-(1H-pyrrolo[2,3-b]pyridine-3-ylmethyl)-3H-spiro[2-benzofuran-1,4′-piperidine]-3′-carboxamide (Example 57);
(cis-1,3′)-N-Cyclopropyl-5,6-difluoro-N-[(4-fluoro-1-naphthyl)methyl]-3H-spiro[2-benzofuran-1,4′-piperidine]-3′-carboxamide (Example 58)
(cis-1,3′)-N-[2-Chloro-3-(trifluoromethyl)benzyl]-N-cyclopropyl-5,6-difluoro-3-oxo-3H-spiro[2-benzofuran-1,4′-piperidine]-3′-carboxamide (Example 59);
(cis-1,3′)-N-Cyclopropyl-5,6-difluoro-N-[2-methyl-3-(trifluoromethyl)benzyl]-3-oxo-3H-spiro[2-benzofuran-1,4′-piperidine]-3′-carboxamide (Example 60); and
(cis-1,3′)-N-Cyclopropyl-5,6-difluoro-3-oxo-N-[2-(trifluoromethyl)benzyl]-3H-spiro[2-benzofuran-1,4′-piperidine]-3′-carboxamide (Example 61).

The general terms used hereinbefore in Formula (I) and hereinafter have, within this disclosure, the following meanings, unless otherwise indicated. Where the plural form is used for compounds, salts, pharmaceutical compositions, diseases and the like, this is intended to mean also a single compound, salt, or the like.

Structural depictions of compounds may show a terminal methyl group as “—CH₃”, “CH₃”, “-Me”, “Me” or “ [symbol] -” (i.e., these have equivalent meanings). A terminal ethyl group may be depicted as “—CH₂CH₃”, “CH₂CH₃”, “-Et”, “Et” or“ [symbol] ” (i.e., these have equivalent meanings).

The term “alkyl”, alone or in combination with other groups, unless indicated otherwise, means saturated, straight and branched chain groups with one to six carbon atoms (which may be represented by “C₁₋₆ alkyl”). When the intended meaning is other than this, for example, when the number of carbon atoms is in the range of one to four carbon atoms, this meaning is represented in like fashion as “C₁₋₄ alkyl”. Example of alkyl groups are methyl, ethyl, n-propyl, iso-propyl, n-butyl, n-pentyl, n-hexyl and etc.

The term “alkenyl”, alone or in combination with other groups, unless indicated otherwise, means unsaturated (i.e. having at least one double bond) straight and branch chain groups with two to six carbon atoms (which may be represented by C₂₋₆ alkenyl). When the intended meaning is other than this, for example, when the number of carbon atoms is in the range of two to four carbon atoms, this meaning is represented in like fashion as C₂₋₄ alkenyl.

The term “alkoxy”, alone or in combination with other groups, refers to an R-0 group, wherein R is an alkyl group. Example of alkoxy groups are methoxy, ethoxy, propoxy, iso-propoxy, isobutoxy, tert-butoxy and etc.

The term “halogen” means fluorine, chlorine, bromine or iodine. In specific embodiments, halogen is fluorine, chlorine or bromine. In particular embodiments, halogen is fluorine or chlorine.

The term “cycloalkyl”, alone or in combination with other groups, unless indicated otherwise, means a saturated cyclic hydrocarbon ring system with three to six carbon atoms, e.g. cyclopropyl, cyclobutyl, cyclopentyl or cyclohexyl. This may be represented by “C₃₋₆ cycloalkyl”.

The term “alkanoyl”, alone or in combination with other groups, unless indicated otherwise, refers to saturated, straight and branched chain groups with one to six carbon atoms with one of the said, unbranching carbon being a carbonyl group (i.e. C═O). This may be represented by “C₁₋₆ alkanoyl”. Examples of alkanoyl groups are —(C═O)H, —(C═O)CH₃, —CH₂(C═O)H, —(C═O)CH₂CH₃, —CH₂(C═O)CH₃ and etc.

The term “carboxy”, alone or in combination with other groups, unless indicated otherwise, refers to —(C═O)O—.

The term “carbocycle” (and variations thereof such as “carbocyclic” or “carbocyclyl”) are used as herein, unless otherwise indicated, refers to a C₃ to C₈ monocyclic saturated or unsaturated ring. The carbocycle may be attached to the rest of the molecule at any carbon atom which results in a stable compound. Saturated carbocyclic rings are also referred to as cycloalkyl rings.

The term “monocycle” (and variations thereof such as “monocyclic”) as used herein refers to a single ring which may be substituted or unsubstituted with one or more substituents as described herein.

The term heterocycle” (and variations thereof such as “heterocyclic” or “heterocyclyl”) broadly refers to a stable four- to eight-membered, saturated or unsaturated monocyclic ring which contains one or more heteroatoms selected from N, O, and S and a balance of carbon atoms); wherein any one or more of the nitrogen and sulfur atoms is optionally oxidized, and any one or more of the nitrogen heteroatoms is optionally quaternized. Unless otherwise specified, when the heterocyclic ring has substituents, it is understood that the substituents may be attached to any atom in the ring, whether a heteroatom or a carbon atom, provided that a stable chemical structure results.

The term “aryl”, alone or in combination, relates to a phenyl, naphthyl or indanyl group. In specific embodiments, the “aryl” is phenyl.

The term “heteroaryl”, alone or in combination, means six-membered aromatic rings containing one to four nitrogen atoms; benzofused six-membered aromatic rings containing one to four nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing two heteroatoms independently selected from oxygen, nitrogen and sulfur and benzofused derivatives of such rings; five-membered aromatic rings containing three nitrogen atoms and benzofused derivatives there of; a tetrazolyl ring, a thiazinyl ring; or coumarinyl. Examples of such ring systems are furanyl, thienyl, pyrrolyl, pyridinyl, pyrimidinyl, indolyl, quinolinyl, isoquinolinyl, imidazolyl, triazinyl, thiazolyl, isothiazolyl, pyridazinyl, pyrazolyl, oxazolyl, isoxazolyl, benzothienyl, quinazolinyl, quinoxalinyl and etc.

The present invention also encompasses a pharmaceutical formulation comprising a pharmaceutically acceptable carrier and the compound of Formula (I) or a pharmaceutically acceptable crystal form or hydrate thereof.

In the compounds of generic Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula (I) can be prepared without undue experimentation by conventional techniques well known to those skilled in the art or by processes analogous to those described in the Schemes and Examples herein using appropriate isotopically-enriched reagents and/or intermediates.

Renin inhibitors, such as those disclosed herein, can be used for the treatment of essential hypertension. Compounds disclosed here are orally-bioavailable and thus should ideally be dosed orally. Alternative modes of administration such as through skin (e.g. transdermal), mucosal membranes (e.g. inhaler, lozenge, suppository) can also be employed when appropriate.

Because the compounds of the present invention inhibit renin, they are useful for blood pressure regulation and indications in which renin inhibition may be useful. Such indications include reduction of intra-ocular pressure, treatment of hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, anxiety states and cognitive disorders.

Compounds of Formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholytics, beta-adrenergic antagonists, alpha-adrenergic antagonists or with other drugs beneficial for the prevention or the treatment of the above-mentioned diseases.

Compounds of Formula (I), optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, mucosally (including sublingual, buccal, rectal, nasal or vaginal administrations), parenterally (including subcutaneous injection, bolus injection, intra-arterial, intravenous, intramuscular, intrasternal injection or infusion administration techniques), by inhalation spray, transdermal, such as passive or iontophoretic delivery, or topical administration, in the form of a unit dosage of a pharmaceutical composition containing an effective amount of the compound and conventional non-toxic pharmaceutically-acceptable carriers, adjuvants and vehicles. Examples of dosage forms include, but are not limited to: tablets, caplets, capsules, such as soft elastic gelatin capsules, cachets, troches, lozenges, dispersions, suppositories, ointments, cataplasms (poultices), pastes, powders, dressings, creams, plasters, solutions, patches, aerosols (e.g., nasal sprays or inhalers), gels, liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs, liquid dosage forms suitable for parenteral administration to a patient, and sterile solids (e.g., crystalline or amorphous solids) that can be reconstituted to provide liquid dosage forms suitable for parenteral administration to a patient. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

### Methods of Synthesis

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvent such as THF, DMF, Et₂O, DME and toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times given are for illustration only. The structure and purity of all final products were ascertained by TLC, mass spectrometry, ¹H NMR and high-pressure liquid chromatography (HPLC). Chemical symbols have their usual meanings. The following abbreviations have also been used: v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)). Unless otherwise specified, all variables mentioned below have the meanings as provided above.

Generally, compounds of the present invention can be prepared via the alkylation of an appropriately functionalized spirocycle amide II; itself readily synthesized from spirocycle ester I via, for example, treatment with amine III in the presence of iPrMgCl, with an appropriately substituted alkylating agent IV (LG=leaving group). Removal of the BOC-protecting group from the resulting spirocycle amide V (Scheme 1) would then furnish spirocycle piperidine VI. Alternatively, spirocycle ester I can also be directly converted into spirocycle amide V by reacting it with an appropriately functionalized amine VII in the presence of a suitable mediator such as iPrMgCl or AlMe₃.

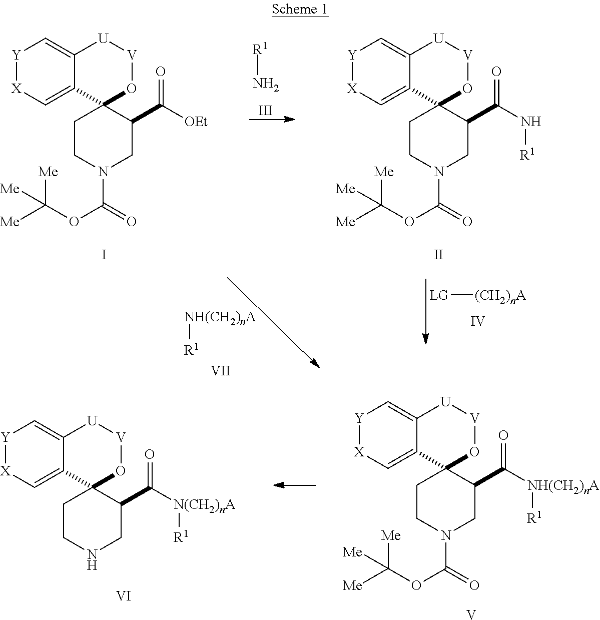

Compounds of the present invention can also be accessed via an initial amide formation between amine VII and α-ketoester VIII, followed by the addition of Grignard reagent derived from an appropriately functionalized arene onto α-ketoamide IX. After the unmasking of a handle suitable for spirocyclization in tertiary alcohol X (PG=protecting group), subsequent intramolecular ring closure with amide XI (FG=functional group) would then furnish spirocycle amide XII. Finally, BOC removal can be accomplished under typical conditions (Scheme 2).

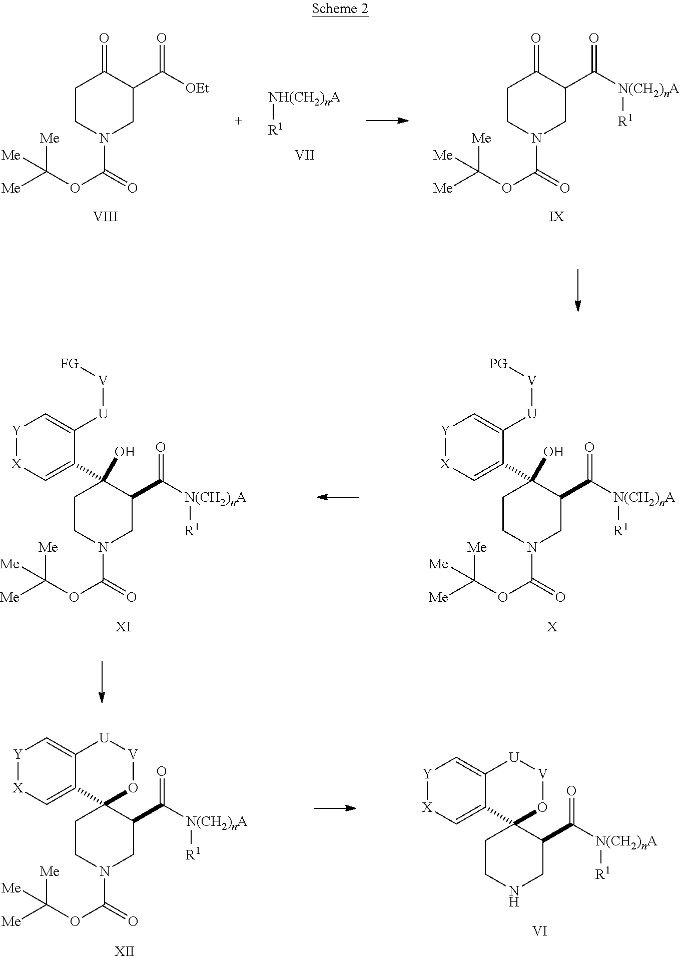

Representative compounds of the invention can be synthesized in accordance with the general synthetic scheme above and are illustrated in the examples that follow. The methods for preparing the various starting materials used in the schemes and examples are well within the knowledge of persons skilled in the art.

### Intermediate 1

### tert-Butyl (1R,3′S)-3′-[(cyclopropylamino)carbonyl]-5,6-difluoro-1′H,3H-spiro[2-benzofuran-1,4′-piperidine]-1′-carboxylate

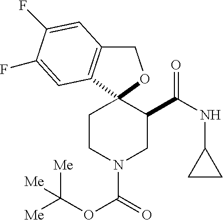

### Step 1

### (2-Bromo-4,5-difluorophenyl)methanol

To a refluxing THF solution (0.42 M) of 2-bromo-4,5-difluorobenzoic acid (1 eq.) was added dropwise neat borane-methyl sulfide complex (1.25 eq.) over a period of 30 min. The resulting solution was heated at reflux for another 2 h before the crude reaction mixture was diluted with ether and carefully quenched with 10% aq. HCl. The aqueous wash was then separated and back-extracted with ether. The combined organic extracts were then washed sequentially with 1 N aq. NaOH, water and brine. The organic extract was dried over Na₂SO₄, filtered and the filtrate concentrated in vacuo to furnish the title compound as a white solid.

### Step 2

### [(2-Bromo-4,5-difluorobenzyl)oxy](tert-butyl)dimethylsilane

To a DMF solution (0.42 M) of (2-bromo-4,5-difluorophenyl)methanol (1 eq.) from the previous step and tert-butyl(chloro)dimethylsilane (1.1 eq.) was added imidazole (1.5 eq.) in one rapid portion. The resulting solution was stirred at RT for 14 h before the crude reaction mixture was diluted with hexanes and washed sequentially with water, 10% aq. HCl, 2 N aq. NaOH, water and brine. The organic extract was dried over Na₂SO₄, filtered and the filtrate concentrated in vacuo to furnish the title compound as a colorless oil.

### Step 3

### 1-tert-Butyl 3-ethyl 4-[2-({[tert-butyl(dimethyl)silyl]oxy}methyl)-4,5-difluorophenyl]-5,6-dihydro-1,3(2H)-pyridinedicarboxylate

To a 2:1 (v/v) toluene: ethanol solution (0.15 M) of 1-(1,1-dimethylethyl) 3-ethyl 4-(4,4,5,5-tetramethyl-1,3,2-dioxaborolan-2-yl)-5,6-dihydro-1,3(2H)-pyridinedicarboxylate (1 eq.) and [(2-bromo-4,5-difluorobenzyl)oxy](tert-butyl)dimethylsilane (1.2 eq.) from the previous step was added sodium carbonate (2 M aq. solution, 3 eq.). The suspension was evacuated and back-filled with N₂. Finally, [1,1′-bis(diphenylphosphino)ferrocene]dichloropalladium(II) (0.03 eq.) was added in one rapid portion and the reaction suspension was heated at 80° C. for 14 h. The reaction was then quenched with the addition of ether and sat. aq. NH₄Cl. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, Hex→1:1 (v/v) Hex:EtOAc) afforded the title compound as a colorless oil that solidified upon standing.

### Step 4

### 1′-tert-Butyl 3′-ethyl (1R,3′5)-5,6-difluoro-1′H,3H-spiro[2-benzofuran-1,4′-piperidine]-1′,3′-dicarboxylate

To a THF solution (0.11 M) of 1-tert-butyl 3-ethyl 4-[2-({[tert-butyl(dimethyl)silyl]oxy}methyl)-4,5-difluorophenyl]-5,6-dihydro-1,3(2H)-pyridinedicarboxylate (1 eq.) from the previous step was added TBAF (1 M THF solution, 1.5 eq.). The resulting mixture was then stirred at RT for 2 h. The volatiles were subsequently removed in vacuo and the resulting residue was partitioned between ether and 10% aq. HCl. The organic layer was separated and washed further with water and brine, dried over Na₂SO₄, filtered and the filtrate concentrated in vacuo. Purification of the crude product thus obtained by way of column chromatography (SiO₂, 9:1 (v/v) Hex:EtOAc→3:7 (v/v) Hex:EtOAc) and subsequent chiral separation (Chiralpak AD, 80:10:10:0.25 (v/v/v/v) Hex: MeOH: iPrOH: triethylamine, slower eluting fraction) afforded the title compound as a white solid.

### Step 5

### Intermediate 1

To a THF solution (0.15 M) of cyclopropylamine (3 eq.) was added isopropyl magnesium chloride (2 M THF solution, 3.5 eq.) at 0 dropwise. After 20 min, F-tert-butyl 3′-ethyl (1R,3′S)-5,6-difluoro-1′H,3H-spiro[2-benzofuran-1,4′-piperidine]-1′,3′-dicarboxylate (1 M THF solution, 1 eq.) from the previous step was then added. The reaction mixture thus obtained was allowed to warm slowly to RT over 14 h before it was quenched with sat. aq. NH₄Cl. The aqueous layer was separated and back-extracted with EtOAc. The combined organic extracts were washed further with water and brine, dried over Na₂SO₄, filtered and the filtrate concentrated in vacuo. Purification of the crude product thus obtained by way of column chromatography (SiO₂, 3:2 (v/v) Hex:EtOAc→EtOAc) afforded the title compound as a white solid.

### Intermediate 2

### tert-Butyl (1R,3′S)-3′-[(cyclopropylamino)carbonyl]-5,6-difluoro-3-oxo-1′H,3H-spiro[2-benzofuran-1,4′-piperidine]-1′-carboxylate

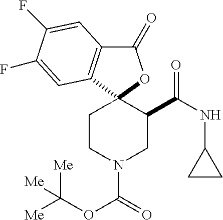

To an acetonitrile solution (0.15 M) of Intermediate 1 (1 eq.) and ruthenium trichloride (0.03 eq.) was added sodium bromate (0.13 M aq. solution, 1.3 eq.). After 8 h, the reaction mixture was diluted with water and EtOAc. The aqueous layer was separated and back-extracted with EtOAc. The combined organic extracts were washed sequentially with 10% aq. Na₂SO₃, 10% aq. HCl, 1 N aq. NaOH, water and brine, dried over Na₂SO₄, filtered and the filtrate concentrated in vacuo. Purification of the crude product thus obtained by way of column chromatography (SiO₂, 4:1 (v/v) Hex:EtOAc→EtOAc) afforded the title compound as a white solid.

The Grignard reagents in Table 1 were synthesized as follows.

TABLE 1

| COMPOUND | STRUCTURE |
|---|---|
| GRIGNARD 1 | 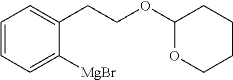 |
| GRIGNARD 2 | 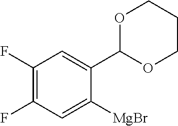 |
| GRIGNARD 3 | 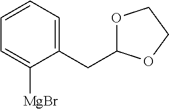 |

### Grignard 1

### Bromo {2-[2-(tetrahydro-2H-pyran-2-yloxy)ethyl]phenyl}magnesium

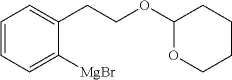

### Step 1

### 2-[2-(2-Bromophenyl)ethoxy]tetrahydro-2H-pyran

To a dichloromethane solution (0.5 M) of 2-(2-bromophenyl)ethanol (1 eq.) and 3,4-dihydro-2H-pyran (1.1 eq.) was added PPTS (0.05 eq.) and the resulting solution was stirred at RT for 14 h. The volatiles were then removed in vacuo and the resulting solution was partitioned between ether and 1 N aq. NaOH. The aqueous layer was separated and back-extracted with ether. The combined organic extracts were washed further with water and brine, dried over Na₂SO₄ and filtered. Concentration of the filtrate thus obtained in vacuo afforded the title compound as a colorless oil.

### Step 2

### Grignard 1

2-[2-(2-Bromophenyl)ethoxy]tetrahydro-2H-pyran (0.5 M THF solution, 1 eq.) from the previous step was added to a THF suspension (0.18 M) of freshly-activated magnesium turnings (2 eq.) and iodine (0.02 eq.) at a rate as to maintain a gentle reflux. The resulting dark suspension was heated at reflux for another 30 min before unreacted magnesium turnings were removed via filtration. The concentration of the title compound in THF thus obtained was determined via titration to be 0.11 M.

### Grignard 2

### Bromo[2-(1,3-dioxan-2-yl)-4,5-difluorophenyl]magnesium

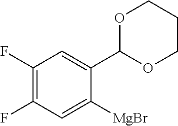

### Step 1

### 2-Bromo-4,5-difluorobenzaldehyde

To a dichloromethane suspension (0.2 M) of (2-bromo-4,5-difluorophenyl)methanol (1 eq., Intermediate 1, Step 1) and sodium bicarbonate (1.5 eq.) was added DMP (1.2 eq.) in one rapid portion. The resulting suspension was stirred at RT for 2 h before it was diluted with ether and washed sequentially with 5% aq. NaHSO₃, 1 N aq. NaOH, water and brine. The organic extract was then dried over Na₂SO₄, filtered through a pad of SiO₂ and the filtrate concentrated in vacuo. Purification of the crude product thus obtained by way of column chromatography (SiO₂, Hex→1:1 (v/v) Hex:EtOAc) afforded the title compound as a white solid.

### Step 2

### 2-(2-Bromo-4,5-difluorophenyl)-1,3-dioxane

To a reaction vessel containing a benzene solution (0.14 M) of 2-bromo-4,5-difluorobenzaldehyde (1 eq.) from the previous step, 1,3-propanediol (1 eq.) and TsOH (0.05 eq.) was attached a Dean-Stark apparatus. The reaction mixture was then heated at reflux. After 20 h the reaction mixture was cooled to RT, diluted with hexanes and ether, and washed sequentially with 1 N aq. NaOH, water and brine. The organic extract was then dried over Na₂SO₄, filtered through a pad of SiO₂ and the filtrate concentrated in vacuo. Purification of the crude product thus obtained by way of column chromatography (SiO₂, Hex→3:1 (v/v) Hex:EtOAc) afforded the title compound as a colorless oil that solidified upon standing.

### Step 3

### Grignard 2

2-(2-Bromo-4,5-difluorophenyl)-1,3-dioxane (0.2 M THF solution, 1 eq.) from the previous step was added to a 1 M THF suspension of Rieke magnesium (2 eq.) at a rate as to keep the internal reaction temperature below 45° C. The resulting dark suspension was then allowed to stir at RT for 3 h before unreacted Rieke magnesium was removed via filtration. The concentration of the title compound in THF thus obtained was determined via titration to be 0.076 M.

### Grignard 3

### Bromo[2-(1,3-dioxolan-2-ylmethyl)phenyl]magnesium

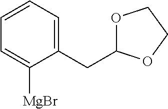

2-(2-Bromobenzyl)-1,3-dioxolane (0.3 M THF solution, 1 eq.) was added to a THF suspension (0.23 M) of freshly-activated magnesium turnings (2 eq.) and iodine (0.02 eq.) at a rate as to maintain a gentle reflux. The resulting dark suspension was heated at reflux for another 30 min before unreacted magnesium turnings were removed via filtration. The concentration of the title compound in THF thus obtained was determined via titration to be 0.21 M.

The alkylation reagents in Table 2 were synthesized as follows.

TABLE 2

| COMPOUND | STRUCTURE |
|---|---|
| ALKYLATION REAGENT 1 | 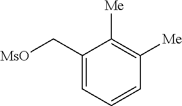 |
| ALKYLATION REAGENT 2 | 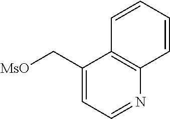 |
| ALKYLATION REAGENT 3 | 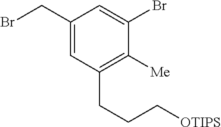 |
| ALKYLATION REAGENT 4 | 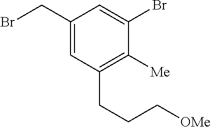 |
| ALKYLATION REAGENT 5 | 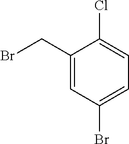 |
| ALKYLATION REAGENT 6 | 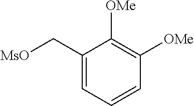 |
| ALKYLATION REAGENT 7 | 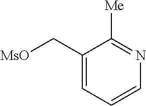 |
| ALKYLATION REAGENT 8 | 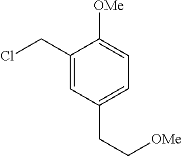 |
| ALKYLATION REAGENT 9 | 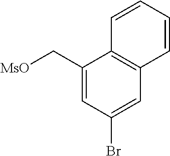 |
| ALKYLATION REAGENT 10 | 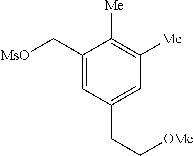 |
| ALKYLATION REAGENT 11 | 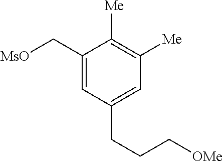 |
| ALKYLATION REAGENT 12 | 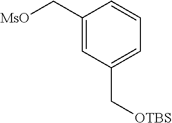 |
| ALKYLATION REAGENT 13 | 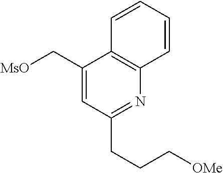 |
| ALKYLATION REAGENT 14 | 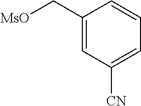 |
| ALKYLATION REAGENT 15 | 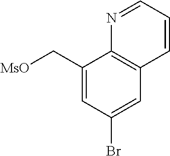 |
| ALKYLATION REAGENT 16 | 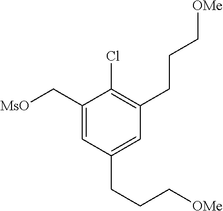 |
| ALKYLATION REAGENT 17 | 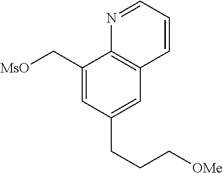 |
| ALKYLATION REAGENT 18 | 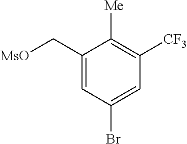 |
| ALKYLATION REAGENT 19 | 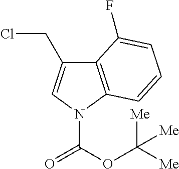 |
| ALKYLATION REAGENT 20 | 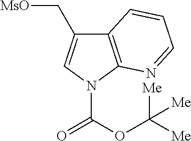 |

### Alkylation Reagent 1

### 2,3-Dimethylbenzyl methanesulfonate

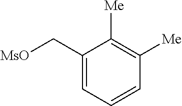

### Step 1

### (2,3-Dimethylphenyl)methanol

To a refluxing THF solution (0.5 M) of 2,3-dimethylbenzoic acid (1 eq.) was added dropwise neat borane-methyl sulfide complex (1.25 eq.) over a period of 10 min. After the completion of addition, the resulting mixture was heated at reflux for another 2 h. The reaction mixture was then cooled to RT, diluted with ether and carefully quenched with 10% aq. HCl. The aqueous layer was separated and back-extracted with ether. The combined organic extracts were washed further with 1 N aq. NaOH and brine, dried over Na₂SO₄, filtered and the filtrate concentrated in vacuo. Further purification of the crude product thus obtained by way of column chromatography (SiO₂, CH₂Cl₂) afforded the title compound as a white solid.

### Step 2

### Alkylation Reagent 1

To a dichloromethane solution (0.2 M) of (2,3-dimethylphenyl)methanol (1 eq.) from the previous step was added triethylamine (1.2 eq.) and then methanesulfonyl chloride (1.1 eq.) at 0° C. The reaction mixture was stirred at 0° C. for 20 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound as a colorless oil.

### Alkylation Reagent 2

### 4-Quinolinylmethyl methanesulfonate

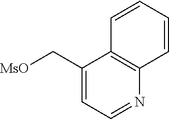

To a dichloromethane solution (0.2 M) of 4-quinolinylmethanol (1 eq.) was added triethylamine (1.2 eq.) and then methanesulfonyl chloride (1.1 eq.) at 0° C. The reaction mixture was stirred at 0° C. for 15 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound as a pale yellow oil.

### Alkylation Reagent 3

### {3-[3-Bromo-5-(bromomethyl)-2-methylphenyl]propoxy}(triisopropyl)silane

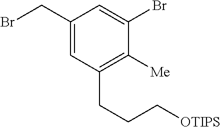

### Step 1

### Methyl 3-bromo-5-[(1E)-3-methoxyprop-1-en-1-yl]-4-methylbenzoate

To a DMF solution (0.1 M) of methyl 3,5-dibromo-4-methylbenzoate (1 eq.) and 4,4,5,5-tetramethyl-2-[(1E)-3-(methyloxy)-1-propen-1-yl]-1,3,2-dioxaborolane (1.1 eq.) was added trans-bis(triphenylphosphine) palladium(II) bromide (0.05 eq.). The vessel was repeatedly evacuated and back-filled with nitrogen. Finally, Na₂CO₃ (2 M aq. solution, 3 eq.) was added and the resulting mixture was heated at 100° C. for 2 h. The now black suspension was cooled to RT, diluted with water and extracted with ether. The combined organic extracts were washed further with water and brine, dried over Na₂SO₄, filtered and the filtrate concentrated in vacuo. Purification of the crude product by way of flash chromatography (SiO₂, 9:1 (v/v) Hex:EtOAc→1:1 (v/v) Hex:EtOAc) afforded the title compound as a colorless oil.

### Step 2

### Methyl 3-bromo-5-(3-methoxypropyl)-4-methylbenzoate

To a refluxing toluene solution (0.1 M) of methyl 3-bromo-5-[(1E)-3-methoxyprop-1-en-1-yl]-4-methylbenzoate (1 eq.) from the previous step was added portionwise benzenesulfonyl hydrazide (6 eq.) over 2 h. After heating at reflux for another hour, the now black reaction suspension was cooled to RT, quenched with sat. aq. NaHCO₃ and extracted with ether. The combined organic extracts were then washed with brine, dried over Na₂SO₄, filtered and the filtrate concentrated in vacuo. Purification of the crude product by way of flash chromatography (SiO₂, 9:1 (v/v) Hex:EtOAc→1:1 (v/v) Hex:EtOAc) afforded the title compound as a colorless oil.

### Step 3

### Methyl 3-bromo-5-(3-hydroxypropyl)-4-methylbenzoate

To a chloroform solution (0.1 M) of methyl 3-bromo-5-(3-methoxypropyl)-4-methylbenzoate (1 eq.) from the previous step was added iodotrimethylsilane (6 eq.). The resulting red solution was stirred at RT in darkness for 18 h. The reaction was quenched with methanol before the volatiles were removed in vacuo. The resulting residue was then partitioned between ether and 1 N aq. NaOH. The aqueous layer was separated and back-extracted with ether. The combined organic extracts were washed further with water and brine, dried over Na₂SO₄, filtered and the filtrate concentrated in vacuo. Purification of the crude product by way of flash chromatography (SiO₂, 9:1 (v/v) Hex:EtOAc→3:7 (v/v) Hex:EtOAc) afforded the title compound as a colorless oil.

### Step 4

### Methyl 3-bromo-4-methyl-5-{3-[(triisopropylsilyl)oxy]propyl}benzoate

To a DMF solution (0.17 M) of methyl 3-bromo-5-(3-hydroxypropyl)-4-methylbenzoate (1 eq.) from the previous step was added imidazole (1.5 eq.) and chlorotriisopropylsilane (1.1 eq.). The resulting solution was allowed to stir at RT for 16 h. The crude reaction mixture was then diluted with hexanes and washed sequentially with water, 10% aq. HCl, 1 N aq. NaOH, water and brine. The organic extract was dried over Na₂SO₄ and filtered. Concentration of the filtrate in vacuo afforded the title compound as a colorless oil.

### Step 5

### (3-Bromo-4-methyl-5-{3-[(triisopropylsilyl)oxy]propyl}phenyl)methanol

To a dichloromethane solution (0.08 M) of methyl 3-bromo-4-methyl-5-{3-[(triisopropylsilyl)oxy]propyl}benzoate (1 eq.) from the previous step was added DIBA1-H (1.5 M toluene solution, 1.2 eq.) dropwise at −78° C. The resulting solution was allowed to warm slowly to RT over 2 h. The reaction mixture was then diluted with ether and carefully quenched with 10% HCl. The organic layer was separated, washed further with 1 N aq. NaOH, water and brine, dried over Na₂SO₄, filtered and the filtrate concentrated in vacuo. Purification of the crude product by way of flash chromatography (SiO₂, 9:1 (v/v) Hex:EtOAc→1:1 (v/v) Hex:EtOAc) afforded the title compound as a colorless oil.

### Step 6

### {3-[3-Bromo-5-(bromomethyl)-2-methylphenyl]propoxy}(triisopropyl)silane

To a dichloromethane solution (0.2 M) of (3-bromo-4-methyl-5-{3-[(triisopropylsilyl)oxy]propyl}phenyl)methanol (1 eq.) from the previous step was added carbon tetrabromide (2 eq.) and triphenylphosphine (2 eq.). The resulting solution was stirred at RT for 16 h. The volatiles were then removed in vacuo and the resulting residue was triturated with hexanes. The insolubles were removed via filtration through a short pad of silica gel and the filtrate was concentrated in vacuo to furnish the title compound as a colorless oil.

### Alkylation Reagent 4

### 3-[3-Bromo-5-(bromomethyl)-2-methylphenyl]propyl methyl ether

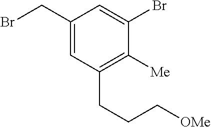

### Step 1

### [3-Bromo-5-(3-methoxypropyl)-4-methylphenyl]methanol

To a dichloromethane solution (0.1 M) of methyl 3-bromo-5-(3-methoxypropyl)-4-methylbenzoate (1 eq., Alkylation Reagent 3, Step 2) was added DIBA1-H (1.5 M toluene solution, 1.2 eq.) dropwise at −78° C. The resulting solution was allowed to warm slowly to RT over 2 h. The reaction mixture was then diluted with ether and carefully quenched with 10% HCl. The organic layer was separated, washed further with 1 N aq. NaOH, water and brine, dried over Na₂SO₄, filtered and the filtrate concentrated in vacuo. Purification of the crude product by way of flash chromatography (SiO₂, 9:1 (v/v) Hex:EtOAc→3:7 (v/v) Hex:EtOAc) afforded the title compound as a colorless oil.

### Step 2

### 3-[3-Bromo-5-(bromomethyl)-2-methylphenyl]propyl methyl ether

To a dichloromethane solution (0.2 M) of [3-bromo-5-(3-methoxypropyl)-4-methylphenyl]methanol (1 eq.) from the previous step was added carbon tetrabromide (2 eq.) and triphenylphosphine (2 eq.). The resulting solution was stirred at RT for 3 h. Silica gel and celite were then added and the resulting suspension was filtered. The filtrate was concentrated in vacuo and the crude product thus obtained was directly subjected to column chromatography (SiO₂, Hex→3:7 (v/v) Hex:EtOAc) to afford the title compound as a colorless oil.

### Alkylation Reagent 5

### 4-Bromo-2-(bromomethyl)-1-chlorobenzene

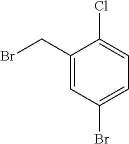

To a dichloromethane solution (0.1 M) of 5-bromo-2-chlorobenzyl alcohol (1 eq.) was added carbon tetrabromide (1.2 eq.), triphenylphosphine (1.7 eq.) and 2,6-lutidine (1 eq.). The resulting solution was stirred at RT for 16 h. The reaction was quenched with sat. aq. NH₄Cl and then extracted with ether. The combined organic extracts were washed further with sat. NaHCO₃, dried over Na₂SO₄, filtered and the filtrate concentrated in vacuo. Purification of the crude product thus obtained via column chromatography (SiO₂, 95:5 (v/v) Hex:EtOAc→4:1 (v/v) Hex:EtOAc) followed by trituration in hexanes afforded the title compound as a white solid.

### Alkylation Reagent 6

### 2,3-Dimethoxybenzyl methanesulfonate

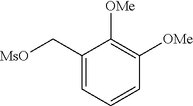

To a dichloromethane solution (0.1 M) of (2,3-dimethoxyphenyl)methanol (1 eq.) was added triethylamine (1.2 eq.) and then methanesulfonyl chloride (1.1 eq.) at 0° C. The reaction mixture was stirred at 0° C. for 3 h before it was quenched with sat. aq. NaHCO₃. The aqueous layer was separated and back-extracted with EtOAc. The combined organic extracts were then dried over Na₂SO₄, filtered and the filtrate concentrated in vacuo to furnish the title compound as a pale yellow oil.

### Alkylation Reagent 7

### (2-Methyl-3-pyridinyl)methyl methanesulfonate

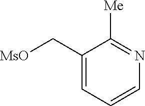

To a dichloromethane solution (0.1 M) of (2-methyl-3-pyridinyl)methanol (1 eq.) was added triethylamine (1.2 eq.) and then methanesulfonyl chloride (1.1 eq.) at 0° C. The reaction mixture was stirred at 0° C. for 3 h before it was quenched with sat. aq. NaHCO₃. The aqueous layer was separated and back-extracted with EtOAc. The combined organic extracts were then dried over Na₂SO₄, filtered and the filtrate concentrated in vacuo to furnish the title compound as a yellow oil.

### Alkylation Reagent 8

### 2-(Chloromethyl)-1-methoxy-4-(2-methoxyethyl)benzene

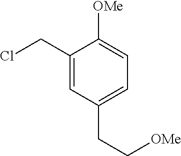

### Step 1

### 2-(4-Methoxyphenyl)ethanol

To a THF solution (0.2 M) of 4-methoxyphenylacetic acid (1 eq.) was added borane-methylsulfide complex (1.25 eq.) dropwise at 0° C. The resulting solution was allowed to warm slowly to RT over 1 h. The reaction mixture was then diluted with ether and carefully quenched with 10% HCl. The organic layer was separated, washed further with 1 N aq. NaOH, dried over Na₂SO₄ and filtered. Concentration of the filtrate in vacuo afforded the title compound as a colorless oil that solidified upon standing.

### Step 2

### 1-Methoxy-4-(2-methoxyethyl)benzene

To a THF solution (0.2 M) of 2-(4-methoxyphenyl)ethanol (1 eq.) from the previous step was added sodium hydride (60% (w/w) dispersion in oil, 1.3 eq.) and iodomethane (1.5 eq.). The resulting solution was stirred at 55° C. for 2 h. The reaction mixture was then cooled to RT and quenched with ice. The aqueous layer was separated and back-extracted with ether. The combined organic extracts were dried over Na₂SO₄, filtered and the filtrate concentrated in vacuo. Purification of the crude product thus obtained via column chromatography (SiO₂, 95:5 (v/v) Hex:EtOAc→7:3 (v/v) Hex:EtOAc) afforded the title compound as a pale yellow oil.

### Step 3

### 2-(Chloromethyl)-1-methoxy-4-(2-methoxyethyl)benzene

To a mixture of 1-methoxy-4-(2-methoxyethyl)benzene (1 eq.) from the previous step, formaldehyde (37% (w/w) aq. solution, 1.2 eq.) and zinc chloride (0.25 eq.) was bubbled HCl gas until saturation. An ice-water bath was used to keep the internal reaction temperature below 40° C. The reaction mixture was then quenched with ice and extracted with dichloromethane. The combined organic extracts were washed further with sat. aq. Na₂CO₃ and water, dried over Na₂SO₄, filtered and the filtrate concentrated in vacuo. Purification of the crude product thus obtained via column chromatography (SiO₂, 95:5 (v/v) Hex:EtOAc→3:2 (v/v) Hex:EtOAc) afforded the title compound as a colorless oil.

### Alkylation Reagent 9

### (3-Bromo-naphthyl)methyl methanesulfonate

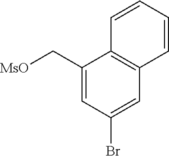

### Step 1

### 5-Bromo-1H,3H-benzo[de]isochromene-1,3-dione

To a nitric acid suspension (0.33 M) of 1H,3H-benzo[de]isochromene-1,3-dione (1 eq.) was added bromine (0.74 eq.) dropwise at 70° C. After 15 min, the now homogeneous reaction mixture was cooled to RT and stirred at RT for 16 h. Water was then added to the reaction mixture and title compound was isolated as a tan solid via filtration.

### Step 2

### 3-Bromo-1-naphthoic acid

To an aqueous suspension (0.2 M) of 5-bromo-1H,3H-benzo[de]isochromene-1,3-dione (1 eq.) from the previous step was added sodium hydroxide (2 M aq. solution, 3 eq.) and yellow mercuric oxide (0.6 M acetic acid solution, 1 eq.). The resulting suspension was heated at reflux for 3 days. The reaction mixture was then cooled to RT and then carefully acidified to a pH of ˜1 with conc. HCl. The resulting mixture was heated again to reflux for another 4 h. Upon cooling to RT, the suspension was diluted further with water before the title compound was isolated as a white solid via filtration.

### Step 3

### Methyl 3-bromo-1-naphthoate

To an ether suspension (0.2 M) of 3-bromo-1-naphthoic acid (1 eq.) from the previous step was added freshly prepared diazomethane (0.5 M ether solution) until no further gaseous evolution could be discerned and a persistent yellow color was obtained. The excess diazomethane was then quenched with a few drops of neat acetic acid. Removal of the volatiles in vacuo furnished the title compound as a pale yellow oil.

### Step 4

### (3-Bromo-1-naphthyl)methanol

To a THF solution (0.5 M) of methyl 3-bromo-1-naphthoate (1 eq.) from the previous step was added lithium aluminum hydride (1 M ether solution, 3 eq.) at 0° C. After 20 min, the excess lithium aluminum hydride was quenched with acetone. The crude reaction mixture was poured into sat. aq. Rochelle's salt and extracted with EtOAc. The combined organic extracts were washed further with water and brine, dried over Na₂SO₄, filtered and the filtrate concentrated in vacuo. Further purification of the crude product thus obtained by way of column chromatography (SiO₂, 9:1 (v/v) Hex:EtOAc→1:9 (v/v) Hex:EtOAc) followed by prep. HPLC (Chiralcel OJ, 7:3 Hex:iPrOH) afforded the title compound as a white solid.

### Step 5

### (3-Bromo-naphthyl)methyl methanesulfonate

To a dichloromethane solution (0.1 M) of (3-bromo-1-naphthyl)methanol (1 eq.) from the previous step was added triethylamine (1.2 eq.) and then methanesulfonyl chloride (1.1 eq.) at 0° C. The reaction mixture was stirred at 0° C. for 3 h before it was quenched with sat. aq. NaHCO₃. The aqueous layer was separated and back-extracted with EtOAc. The combined organic extracts were then dried over Na₂SO₄, filtered and the filtrate concentrated in vacuo to furnish the title compound as a white solid.

### Alkylation Reagent 10

### 5-(2-Methoxyethyl)-2,3-dimethylbenzyl methanesulfonate

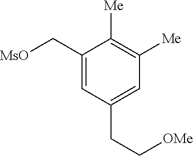

### Step 1

### 5-Bromo-2,3-dimethylbenzoic acid

To an acetic acid solution (0.2 M) of 2,3-dimethylbenzoic acid (1 eq.), nitric acid (12 eq.) and bromine (1.1 eq.) was added dropwise silver nitrate (2.5 M aq. solution, 1.3 eq.) over a period of 30 min. After 1 h of stirring at RT, the reaction mixture was diluted with water and extracted with EtOAc. The combined organic extracts were washed further with brine, dried over Na₂SO₄, filtered and the filtrate concentrated in vacuo. Concentration of the filtrate in vacuo and trituration of the crude product thus obtained in hexanes afforded the title compound as an off-white solid.

### Step 2

### Methyl 5-bromo-2,3-dimethylbenzoate

To a THF solution (1 M) of 5-bromo-2,3-dimethylbenzoic acid (1 eq.) from the previous step was added freshly prepared diazomethane (0.5 M ether solution) until no further gaseous evolution could be discerned and a persistent yellow color was obtained. The excess diazomethane was then quenched with a few drops of neat acetic acid. Removal of the volatiles in vacuo furnished the title compound as a pale yellow oil.

### Step 3

### Methyl 2,3-dimethyl-5-vinylbenzoate

To a DMF solution (0.3 M) of methyl 5-bromo-2,3-dimethylbenzoate (1 eq.) from the previous step was added vinyl tri-n-butyl tin (1.2 eq.) and tetrakis(triphenylphosphine)palladium (0.05 eq.). The resulting mixture was degassed with nitrogen before it was heated 80° C. for 16 h. After cooling to RT, the reaction mixture was diluted with water and extracted with ether. The combined organic extracts were then treated with 10% aq. NaF for 18 h, washed further with water and brine, dried over Na₂SO₄, filtered and the filtrate concentrated in vacuo. Purification of the crude product thus obtained via column chromatography (SiO₂, 95:5 (v/v) Hex:EtOAc→4:1 (v/v) Hex:EtOAc) afforded the title compound as a colorless oil.

### Step 4

### Methyl 5-(2-hydroxyethyl)-2,3-dimethylbenzoate

To a THF solution (0.15 M) of 9-BBN dimmer (1.5 eq.) was added methyl 2,3-dimethyl-5-vinylbenzoate (1 eq.) from the previous step and the resulting mixture was allowed to stir at RT for 16 h. At −30° C., the reaction was quenched with the sequential addition of H₂O₂ (30% (w/w) aq. solution, 3 eq.) and sodium hydroxide (3 N aq. solution, 1.6 eq.). After 1 h of stirring at −10° C., the mixture was diluted further with water and extracted with ether. The combined organic extracts were washed further with water and brine, dried over Na₂SO₄, filtered and the filtrate concentrated in vacuo. Purification of the crude product thus obtained via column chromatography (SiO₂, 9:1 (v/v) Hex:EtOAc→1:2 (v/v) Hex:EtOAc) afforded the title compound as a white solid.

### Step 5

### Methyl 5-(2-methoxyethyl)-2,3-dimethylbenzoate

To a THF solution (0.15 M) of methyl 5-(2-hydroxyethyl)-2,3-dimethylbenzoate (1 eq.) from the previous step was added sodium hydride (60% (w/w) dispersion in oil, 1.3 eq.) and iodomethane (1.5 eq.). The resulting solution was stirred at 55° C. for 2 h. The reaction mixture was then cooled to RT and quenched with ice. The aqueous layer was separated and back-extracted with ether. The combined organic extracts were dried over Na₂SO₄, filtered and the filtrate concentrated in vacuo. Purification of the crude product thus obtained via column chromatography (SiO₂, 95:5 (v/v) Hex:EtOAc→3:2 (v/v) Hex:EtOAc) afforded the title compound as a white semi-solid.

### Step 6

### [5-(2-Methoxyethyl)-2,3-dimethylphenyl]methanol

To an ether solution (0.15 M) of methyl 5-(2-methoxyethyl)-2,3-dimethylbenzoate (1 eq.) from the previous step was added lithium aluminum hydride (2.2 eq.) at −78° C. The cooling bath was then removed and the resulting mixture was stirred at RT for 3 h. The reaction mixture was then carefully quenched with water and extracted with EtOAc. The combined organic extracts were dried over Na₂SO₄, filtered and the filtrate concentrated in vacuo. Purification of the crude product thus obtained via column chromatography (SiO₂, 95:5 (v/v) Hex:EtOAc→2:3 (v/v) Hex:EtOAc) afforded the title compound as a colorless oil.

### Step 7

### 5-(2-Methoxyethyl)-2,3-dimethylbenzyl methanesulfonate

To a dichloromethane solution (0.1 M) of [5-(2-methoxyethyl)-2,3-dimethylphenyl]methanol (1 eq.) from the previous step was added triethylamine (1.2 eq.) and then methanesulfonyl chloride (1.1 eq.) at 0° C. The reaction mixture was stirred at 0° C. for 3 h before it was quenched with sat. aq. NaHCO₃. The aqueous layer was separated and back-extracted with EtOAc. The combined organic extracts were then dried over Na₂SO₄, filtered and the filtrate concentrated in vacuo to furnish the title compound as a colorless oil.

### Alkylation Reagent 11

### 5-(3-Methoxypropyl)-2,3-dimethylbenzyl methanesulfonate

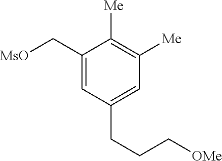

### Step 1

### Methyl 5-[(1E)-3-methoxy-1-propen-1-yl]-2,3-dimethylbenzoate

To a DMF solution (0.15 M) of methyl 5-bromo-2,3-dimethylbenzoate (1 eq., Alkylation Reagent 10, Step 2) and 4,4,5,5-tetramethyl-2-[(1E)-3-(methyloxy)-1-propen-1-yl]-1,3,2-dioxaborolane (1.5 eq.) was added trans-bis(triphenylphosphine) palladium(II) bromide (0.1 eq.). The vessel was repeatedly evacuated and back-filled with nitrogen. Finally, Na₂CO₃ (2 M aq. solution, 3 eq.) was added and the resulting mixture was heated at 90° C. for 14 h. The now black suspension was cooled to RT, quenched with sat. aq. NH₄Cl and extracted with EtOAc. The combined organic extracts were washed further with water and brine, dried over Na₂SO₄, filtered and the filtrate concentrated in vacuo. Purification of the crude product by way of flash chromatography (SiO₂, 95:5 (v/v) Hex:EtOAc→3:2 (v/v) Hex:EtOAc) afforded the title compound.

### Step 2

### Methyl 5-(3-methoxypropyl)-2,3-dimethylbenzoate

To an ethyl acetate solution (0.05 M) of methyl 5-[(1E)-3-methoxy-1-propen-1-yl]-2,3-dimethylbenzoate (1 eq.) from the previous step was added palladium black (10% (w/w) over activated carbon, 0.3 eq.). The vessel was evacuated and back-filled with hydrogen. Under a balloon-maintained hydrogen atmosphere, the reaction suspension was stirred at RT for 3 h. The reaction mixture was then diluted with dichloromethane and filtered through a pad of celite. Concentration of the filtrate in vacuo afforded the title compound.

### Step 3

### [5-(3-Methoxypropyl)-2,3-dimethylphenyl]methanol

To an ether solution (0.15 M) of methyl 5-(3-methoxypropyl)-2,3-dimethylbenzoate (1 eq.) from the previous step was added lithium aluminum hydride (3 eq.) at −78° C. The cooling bath was then removed and the resulting mixture was stirred at RT for 3 h. The reaction mixture was then carefully quenched with water and extracted with ether. The combined organic extracts were dried over Na₂SO₄, filtered and the filtrate concentrated in vacuo. Purification of the crude product thus obtained via column chromatography (SiO₂, 9:1 (v/v) Hex:EtOAc→2:3 (v/v) Hex:EtOAc) afforded the title compound as a colorless oil.

### Step 4

### 5-(3-Methoxypropyl)-2,3-dimethylbenzyl methanesulfonate

To a dichloromethane solution (0.1 M) of [5-(3-methoxypropyl)-2,3-dimethylphenyl]methanol (1 eq.) from the previous step was added triethylamine (1.2 eq.) and then methanesulfonyl chloride (1.1 eq.) at 0° C. The reaction mixture was stirred at 0° C. for 3 h before it was quenched with sat. aq. NaHCO₃. The aqueous layer was separated and back-extracted with EtOAc. The combined organic extracts were then dried over Na₂SO₄, filtered and the filtrate concentrated in vacuo to furnish the title compound as a colorless oil.

### Alkylation Reagent 12

### 3-({[tert-Butyl(dimethyl)silyl]oxy}methyl)benzyl methanesulfonate

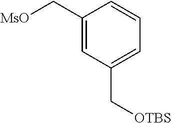

To a dichloromethane solution (0.1 M) of [3-({[tert-butyl(dimethyl)silyl]oxy}methyl)phenyl]methanol (1 eq.) was added triethylamine (1.2 eq.) and then methanesulfonyl chloride (1.1 eq.) at 0° C. The reaction mixture was stirred at 0° C. for 3 h before it was quenched with sat. aq. NaHCO₃. The aqueous layer was separated and back-extracted with EtOAc. The combined organic extracts were then dried over Na₂SO₄, filtered and the filtrate concentrated in vacuo to furnish the title compound as a colorless oil.

### Alkylation Reagent 13

### [2-(3-Methoxypropyl)-4-quinolinyl]methyl methanesulfonate

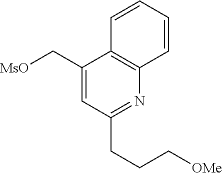

### Step 1

### Methyl 2-[(1E)-3-methoxy-1-propen-1-yl]-4-quinolinecarboxylate

To a DMF solution (0.15 M) of methyl 2-bromo-4-quinolinecarboxylate (1 eq., prepared according to procedure described in WO 2007/009250 A1) and 4,4,5,5-tetramethyl-2-[(1E)-3-(methyloxy)-1-propen-1-yl]-1,3,2-dioxaborolane (1.5 eq.) was added trans-bis(triphenylphosphine) palladium(II) bromide (0.1 eq.). The vessel was repeatedly evacuated and back-filled with nitrogen. Finally, Na₂CO₃ (2 M aq. solution, 3 eq.) was added and the resulting mixture was heated at 100° C. for 3 h. The now black suspension was cooled to RT, quenched with water and extracted with EtOAc. The combined organic extracts were washed further with water and brine, dried over Na₂SO₄, filtered and the filtrate concentrated in vacuo. Purification of the crude product by way of flash chromatography (SiO₂, 85:15 (v/v) Hex:EtOAc→3:7 (v/v) Hex:EtOAc) afforded the title compound as an orange oil.

### Step 2

### Methyl 2-(3-methoxypropyl)-4-quinolinecarboxylate

To a dichloromethane solution (0.2 M) of methyl 2-[(1E)-3-methoxy-1-propen-1-yl]-4-quinolinecarboxylate (1 eq.) from the previous step was added Crabtree's catalyst (0.03 eq.). The vessel was evacuated and back-filled with hydrogen. Under a balloon-maintained hydrogen atmosphere, the reaction mixture was stirred at RT for 18 h. The reaction mixture was then concentrated in vacuo to afford the title compound as an orange oil.

### Step 3

### [2-(3-Methoxypropyl)-4-quinolinyl]methanol

To an ether solution (0.15 M) of methyl 2-(3-methoxypropyl)-4-quinolinecarboxylate (1 eq.) from the previous step was added lithium aluminum hydride (4 eq.) at −78° C. The resulting mixture was then allowed to warm slowly to RT over 18 h. The reaction mixture was then carefully quenched with water and extracted with EtOAc. The combined organic extracts were dried over Na₂SO₄, filtered and the filtrate concentrated in vacuo. Purification of the crude product thus obtained via column chromatography (SiO₂, CH₂Cl₂→95:15 (v/v) CH₂Cl₂: 2.0 M NH₃ in MeOH) afforded the title compound as an orange oil.

### Step 4

### [2-(3-Methoxypropyl)-4-quinolinyl]methyl methanesulfonate

To a dichloromethane solution (0.2 M) of [2-(3-methoxypropyl)-4-quinolinyl]methanol (1 eq.) from the previous step was added triethylamine (1.2 eq.) and then methanesulfonyl chloride (1.1 eq.) at 0° C. The reaction mixture was stirred at 0° C. for 20 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound as a brown oil.

### Alkylation Reagent 14

### 3-cyanobenzyl methanesulfonate

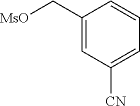

To a dichloromethane solution (0.2 M) of 3-cyanobenzyl alcohol (1 eq.) was added triethylamine (1.2 eq.) and then methanesulfonyl chloride (1.1 eq.) at 0° C. The reaction mixture was stirred at 0° C. for 20 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound as a colorless oil.

### Alkylation Reagent 15

### (6-Bromo-8-quinolinyl)methyl methanesulfonate

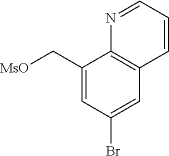

### Step 1

### Methyl 6-bromo-8-quinolinecarboxylate

A sulfuric acid solution (3 M) of 5-bromo-2-nitrobenzoic acid (1 eq.), 2-amino-5-bromobenzoic acid (2 eq.) and glycerol (6 eq.) was heated at reflux for 8 h. After cooling to RT, monomethylglycol was added to the reaction mixture and the crude quinoline acid was isolated via filtration as the corresponding sulfuric acid salt. This salt was then taken up in methanol (1 M). The reaction vessel was sealed and heated to 100° C. for 16 h. The reaction mixture was then cooled to RT and carefully quenched with sat. aq. NaHCO₃. Methanol was then removed in vacuo and the resulting aqueous suspension was extracted with EtOAc. The combined organic extracts were dried over Na₂SO₄, filtered and the filtrate concentrated in vacuo. Purification of the crude product thus obtained via column chromatography (SiO₂, 85:15 (v/v) Hex:EtOAc→2:3 (v/v) Hex:EtOAc) afforded the title compound as a yellow solid.

### Step 2

### (6-Bromo-8-quinolinyl)methanol

To an ether solution (0.15 M) of methyl 6-bromo-8-quinolinecarboxylate (1 eq.) from the previous step was added lithium aluminum hydride (3 eq.) at −78° C. The resulting mixture was then allowed to warm slowly to RT over 4 h. The reaction mixture was then carefully quenched with water and extracted with ether. The combined organic extracts were dried over Na₂SO₄, filtered and the filtrate concentrated in vacuo. Purification of the crude product thus obtained via column chromatography (SiO₂, 4:1 (v/v) Hex:EtOAc→1:4 (v/v) Hex:EtOAc) afforded the title compound as a pale yellow solid.

### Step 3

### (6-Bromo-8-quinolinyl)methyl methanesulfonate

To a dichloromethane solution (0.2 M) of (6-bromo-8-quinolinyl)methanol (1 eq.) was added triethylamine (1.2 eq.) and then methanesulfonyl chloride (1.1 eq.) at 0° C. The reaction mixture was stirred at 0° C. for 20 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound as a pale yellow oil.

### Alkylation Reagent 16

### 2-Chloro-3,5-bis(3-methoxypropyl)benzyl methanesulfonate

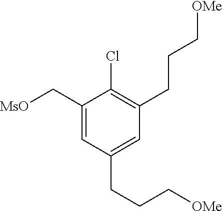

### Step 1

### 3,5-Dibromo-2-chlorobenzoic acid

To a mixture of 2-amino-3,5-dibromobenzoic acid (1 eq.) and HCl (10 M aq. solution, 20 eq.) was added sodium nitrite (4.6 M aq. solution, 1.35 eq.) dropwise at 0° C. Following the completion of addition, the resulting mixture was allowed to stir for 1 h before it was poured into a solution of copper(I) chloride (1 eq.) in concentrated HCl. The resulting foam was allowed to warm slowly to RT and before it was extracted with EtOAc. The combined organic extracts were washed further with water and brine, dried over Na₂SO₄ and filtered. Concentration of the filtrate thus obtained in vacuo afforded the title compound as a beige solid.

### Step 2

### Methyl 3,5-dibromo-2-chlorobenzoate

To a THF solution (1 M) of 3,5-dibromo-2-chlorobenzoic acid (1 eq.) from the previous step was added freshly prepared diazomethane (0.5 M ether solution) until no further gaseous evolution could be discerned and a persistent yellow color was obtained. The excess diazomethane was then quenched with a few drops of neat acetic acid. Removal of the volatiles in vacuo furnished the title compound as a peach solid.

### Step 3

### Methyl 2-chloro-3,5-bis[(1E)-3-methoxyprop-1-en-1-yl]benzoate

To a DMF solution (0.15 M) of methyl 3,5-dibromo-2-chlorobenzoate (1 eq.) and 4,4,5,5-tetramethyl-2-[(1E)-3-(methyloxy)-1-propen-1-yl]-1,3,2-dioxaborolane (2.5 eq.) was added trans-bis(triphenylphosphine) palladium(II) bromide (0.1 eq.). The vessel was repeatedly evacuated and back-filled with nitrogen. Finally, Na₂CO₃ (2 M aq. solution, 5 eq.) was added and the resulting mixture was heated at 100° C. for 16 h. The now black suspension was cooled to RT, diluted with water and extracted with ether. The combined organic extracts were washed further with water and brine, dried over Na₂SO₄, filtered and the filtrate concentrated in vacuo. Purification of the crude product by way of flash chromatography (SiO₂, 9:1 (v/v) Hex:EtOAc→3:7 (v/v) Hex:EtOAc) afforded the title compound as a yellow oil.

### Step 4

### Methyl 2-chloro-3,5-bis(3-methoxypropyl)benzoate

To a refluxing toluene solution (0.1 M) of methyl 2-chloro-3,5-bis[(1E)-3-methoxyprop-1-en-1-yl]benzoate (1 eq.) from the previous step was added portionwise benzenesulfonyl hydrazide (7 eq.) over 4 h. After heating at reflux for another 2 h, the now black reaction suspension was cooled to RT, diluted with EtOAc and washed sequentially with 10% aq. HCl, 1 N aq. NaOH, water and brine. The organic extract was then dried over Na₂SO₄, filtered and the filtrate concentrated in vacuo. Purification of the crude product by way of flash chromatography (SiO₂, 9:1 (v/v) Hex:EtOAc→3:7 (v/v) Hex:EtOAc) afforded the title compound.

### Step 5

### [2-Chloro-3,5-bis(3-methoxypropyl)phenyl]methanol

To an ether solution (0.15 M) of methyl 2-chloro-3,5-bis(3-methoxypropyl)benzoate (1 eq.) from the previous step was added lithium aluminum hydride (3 eq.) at −78° C. The resulting mixture was then allowed to warm slowly to RT over 4 h. The reaction mixture was then carefully quenched with water and extracted with ether. The combined organic extracts were dried over Na₂SO₄, filtered and the filtrate concentrated in vacuo. Purification of the crude product thus obtained via column chromatography (SiO₂, 4:1 (v/v) Hex:EtOAc→1:4 (v/v) Hex:EtOAc) afforded the title compound as a colorless oil.

### Step 6

### 2-Chloro-3,5-bis(3-methoxypropyl)benzyl methanesulfonate

To a dichloromethane solution (0.2 M) of [2-chloro-3,5-bis(3-methoxypropyl)phenyl]methanol (1 eq.) was added triethylamine (1.2 eq.) and then methanesulfonyl chloride (1.1 eq.) at 0° C. The reaction mixture was stirred at 0° C. for 40 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound as a pale yellow oil.

### Alkylation Reagent 17

### [6-(3-Methoxypropyl)-8-quinolinyl]methyl methanesulfonate

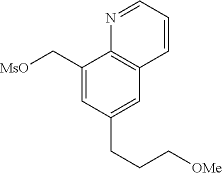

Prepared according to the procedure described in Alkylation Reagent 13 but using methyl 6-bromo-8-quinolinecarboxylate (1 eq., Alkylation Reagent 15, Step 1) in place of methyl 2-bromo-4-quinolinecarboxylate as the starting material in step 1.

### Alkylation Reagent 18

### 5-Bromo-2-methyl-3-(trifluoromethyl)benzyl methanesulfonate

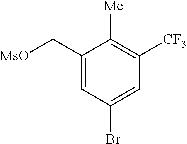

### Step 1

### 5-Bromo-2-methyl-3-(trifluoromethyl)benzoic acid

To an acetic acid solution (0.2 M) of 2-methyl-3-(trifluoromethyl)benzoic acid (1 eq.), nitric acid (10 eq.) and bromine (1.1 eq.) was added dropwise silver nitrate (2.5 M aq. solution, 1.3 eq.) over a period of 30 min. After 3 h of stirring at RT, the reaction mixture was diluted with water and extracted with EtOAc. The combined organic extracts were washed further with brine, dried over Na₂SO₄, filtered and the filtrate concentrated in vacuo. Concentration of the filtrate in vacuo and trituration of the crude product thus obtained in 9:1 (v/v) hexanes:ether afforded the title compound as an off-white solid.

### Step 2

### [5-Bromo-2-methyl-3-(trifluoromethyl)phenyl]methanol

To a refluxing THF solution (0.2 M) of 5-bromo-2-methyl-3-(trifluoromethyl)benzoic acid (1 eq.) from the previous step was added neat borane-methylsulfide complex (1.25 eq.) dropwise over 30 min. After 2 h of stirring at reflux, the mixture was cooled to RT and carefully quenched with 1 M aq. HCl. The aqueous layer was separated and back-extracted with ether. The combined organic extracts were washed further with brine, dried over Na₂SO₄, filtered and the filtrate concentrated in vacuo. Purification of the crude product thus obtained via column chromatography (SiO₂, Hex→1:9 (v/v) Hex:EtOAc) afforded the title compound as a white solid.

### Step 3

### 5-Bromo-2-methyl-3-(trifluoromethyl)benzyl methanesulfonate

To a dichloromethane solution (0.2 M) of [5-bromo-2-methyl-3-(trifluoromethyl)phenyl]methanol (1 eq.) was added triethylamine (1.2 eq.) and then methanesulfonyl chloride (1.1 eq.) at 0° C. The reaction mixture was stirred at 0° C. for 2 h before it was quenched with sat. aq. NaHCO₃. The aqueous layer was separated and back-extracted with ether. The combined organic extracts were then dried over Na₂SO₄, filtered and the filtrate concentrated in vacuo to furnish the title compound.

### Alkylation Reagent 19

### tert-Butyl 3-(chloromethyl)-4-fluoro-1H-indole-1-carboxylate

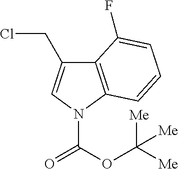

### Step 1

### 4-Fluoro-1H-indole-3-carbaldehyde

To a DMF solution (0.5 M) of 4-fluoro-1H-indole (1 eq.) was added phosphorous oxychloride (1.2 eq.) at 0° C. The resulting mixture was allowed to warm slowly to RT over 18 h. The reaction mixture was quenched with the addition of 2 M aq. NaOH and then extracted with ether. The combined organic extracts were washed further with water and brine, dried over Na₂SO₄ and filtered. Concentration of the filtrate in vacuo furnished the title compound.

### Step 2

### tert-Butyl 4-fluoro-3-formyl-1H-indole-1-carboxylate

To an acetonitrile solution (0.2 M) of 4-fluoro-1H-indole-3-carbaldehyde (1 eq.) from the previous step was added triethylamine (1.05 eq.), DMAP (0.2 eq.) and di-tert-butyl carbonate (1.2 eq.) at 0° C. The resulting mixture was allowed to warm slowly to RT over 18 h. The resulting mixture was then diluted with water and extracted with EtOAc. The combined organic extracts were washed further with sat. aq. NH₄Cl and brine, dried over Na₂SO₄ and filtered. Concentration of the filtrate in vacuo furnished the title compound as a brown solid.

### Step 3

### tert-Butyl 4-fluoro-3-(hydroxymethyl)-1H-indole-1-carboxylate

To a 10:1 (v/v) MeOH: THF solution (0.2 M) of tert-butyl 4-fluoro-3-formyl-1H-indole-1-carboxylate (1 eq.) from the previous step was added sodium borohydride (2 eq.) at 0° C. After 2 h of stirring at 0° C., the mixture was carefully quenched with sat. aq. NH₄Cl and then extracted with EtOAc. The combined organic extracts were washed further with brine, dried over Na₂SO₄, filtered and the filtrate concentrated in vacuo. Purification of the crude product thus obtained via column chromatography (SiO₂, CH₂Cl₂→9:1 (v/v) CH₂Cl₂:MeOH) afforded the title compound as a beige solid.

### Step 4

### tert-Butyl 3-(chloromethyl)-4-fluoro-1H-indole-1-carboxylate

To a dichloromethane solution (0.2 M) of tert-butyl 4-fluoro-3-(hydroxymethyl)-1H-indole-1-carboxylate (1 eq.) was added triethylamine (1.2 eq.) and then methanesulfonyl chloride (1.1 eq.) at 0° C. The reaction mixture was stirred at 0° C. for 2 h before it was quenched with sat. aq. NaHCO₃. The aqueous layer was separated and back-extracted with dichloromethane. The combined organic extracts were then dried over Na₂SO₄, filtered and the filtrate concentrated partially in vacuo. Dilution with THF furnished the title compound as a 0.1 M THF solution.

### Alkylation Reagent 20

### tert-Butyl 3-{[(methylsulfonyl)oxy]methyl}-1H-pyrrolo[2,3-b]pyridine-1-carboxylate

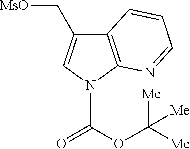

### Step 1

### tert-Butyl 3-formyl-1H-pyrrolo[2,3-b]pyridine-1-carboxylate

To an acetonitrile solution (0.2 M) of 1H-pyrrolo[2,3-b]pyridine-3-carbaldehyde (1 eq.) was added triethylamine (1.05 eq.), DMAP (0.3 eq.) and di-tert-butyl carbonate (1.2 eq.) at 0° C. The resulting mixture was allowed to warm slowly to RT over 18 h. The resulting mixture was then diluted with water and extracted with EtOAc. The combined organic extracts were washed further with sat. aq. NH₄Cl, sat. aq. NaHCO₃ and brine, dried over Na₂SO₄ and filtered. Concentration of the filtrate in vacuo furnished the title compound.

### Step 2

### tert-Butyl 3-(hydroxymethyl)-1H-pyrrolo[2,3-b]pyridine-1-carboxylate

To a 10:1 (v/v) MeOH: THF solution (0.2 M) of tert-butyl 3-formyl-1H-pyrrolo[2,3-b]pyridine-1-carboxylate (1 eq.) from the previous step was added sodium borohydride (2 eq.) at 0° C. After 3 h of stirring at 0° C., the mixture was carefully quenched with sat. aq. NH₄Cl and then extracted with EtOAc. The combined organic extracts were washed further with brine, dried over Na₂SO₄, filtered and the filtrate concentrated in vacuo. Purification of the crude product thus obtained via column chromatography (SiO₂, CH₂Cl₂→9:1 (v/v) CH₂Cl₂:MeOH) afforded the title compound as a pale yellow solid.

### Step 3

### tert-Butyl 3-{[(methylsulfonyl)oxy]methyl}-1H-pyrrolo[2,3-b]pyridine-1-carboxylate

To a dichloromethane solution (0.2 M) of tert-butyl 3-(hydroxymethyl)-1H-pyrrolo[2,3-b]pyridine-1-carboxylate (1 eq.) was added triethylamine (1.2 eq.) and then methanesulfonyl chloride (1.1 eq.) at 0° C. The reaction mixture was stirred at 0° C. for 2 h before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound.

### Example 1

### (cis-1,3′)-N-Cyclopropyl-N-[3-(2-methoxyethoxy)-5-(3-methoxypropyl)benzyl]-3-oxo-3H-spiro[2-benzofuran-1,4′-piperidine]-3′-carboxamide

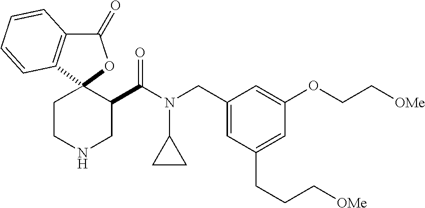

### Step 1

### tert-Butyl 3-({cyclopropyl[3-(2-methoxyethoxy)-5-(3-methoxypropyl)benzyl]amino}carbonyl)-4-oxo-1-piperidinecarboxylate

1-tert-Butyl 3-ethyl 4-oxo-1,3-piperidinedicarboxylate (1 eq.), N-[3-(2-methoxyethoxy)-5-(3-methoxypropyl)benzyl]cyclopropanamine (1 eq., prepared according to the procedure described in WO 2007/009250 A1) and DMAP (0.2 eq.) were heated at 140° C. for 5 h. Purification of the crude product thus obtained by way of column chromatography (SiO₂, 95:5 (v/v) Hex:EtOAc→3:7 (v/v) Hex:EtOAc) followed by swishing in 9:1 (v/v) Hex:Et₂O afforded the title compound as a white solid.

### Step 2

### tert-Butyl(cis-3,4)-3-({cyclopropyl[3-(2-methoxyethoxy)-5-(3-methoxypropyl)benzyl]amino}carbonyl)-4-[2-(1,3-dioxan-2-yl)phenyl]-4-hydroxy-1-piperidinecarboxylate

To a THF solution (0.05 M) of tert-butyl 3-({cyclopropyl[3-(2-methoxyethoxy)-5-(3-methoxypropyl)benzyl]amino}carbonyl)-4-oxo-1-piperidinecarboxylate (1 eq.) from the previous step was added 2-(1,3-dioxan-2-yl)phenyl magnesium bromide (0.25 M THF solution, 2.5 eq.) at RT over a period of 10 min. The resulting dark suspension was allowed to stir at RT for another 18 h. The reaction mixture was then diluted with ether and carefully quenched with 10% aq. HCl. The aqueous layer was separated and back-extracted with ether. The combined organic extracts were washed further with brine, dried over Na₂SO₄, filtered and the filtrate concentrated in vacuo. Purification of the crude product thus obtained by way of column chromatography (SiO₂, 9:1 (v/v) Hex:EtOAc→EtOAc) afforded the title compound as a white solid.

### Step 3

### tert-Butyl(cis-1,3′)-3′-({cyclopropyl[3-(2-methoxyethoxy)-5-(3-methoxypropyl)benzyl]amino}carbonyl)-3-hydroxy-1′H,3H-spiro[2-benzofuran-1,4′-piperidine]-1′-carboxylate

To a 10:1 (v/v) acetone: water solution (0.02 M) of tert-butyl(cis-3,4)-3-({cyclopropyl[3-(2-methoxyethoxy)-5-(3-methoxypropyl)benzyl]amino}carbonyl)-4-[2-(1,3-dioxan-2-yl)phenyl]-4-hydroxy-1-piperidinecarboxylate (1 eq.) from the previous step was added PPTS (0.3 eq.). The resulting solution was heated at 80° C. for 3 h. The volatiles were then removed in vacuo and the resulting residue was partitioned between ether and 1 N aq. NaOH. The aqueous wash was separated and back-extracted with ether. The combined organic extracts were washed further with water and brine, dried over Na₂SO₄ and filtered. Concentration of the filtrate thus obtained afforded the title compound as a white solid.

### Step 4

### tert-Butyl(cis-1,3′)-3′-({cyclopropyl[3-(2-methoxyethoxy)-5-(3-methoxypropyl)benzyl]amino}carbonyl)-3-oxo-1′H,3H-spiro[2-benzofuran-1,4′-piperidine]-1′-carboxylate

To a dichloromethane suspension (0.06 M) of tert-butyl(cis-1,3′)-3′-({cyclopropyl[3-(2-methoxyethoxy)-5-(3-methoxypropyl)benzyl]amino}carbonyl)-3-hydroxy-1′H,3H-spiro[2-benzofuran-1,4′-piperidine]-1-carboxylate (1 eq.) from the previous step, NMO (2.3 eq.) and 4 Å molecular sieves was added TPAP (0.05 eq.). After stirring at RT for 1 h, the reaction mixture was diluted with ether and filtered through a pad of celite. The filtrate was concentrated in vacuo and the crude product thus obtained was immediately subjected to purification by way of column chromatography (SiO₂, 9:1 (v/v) Hex:EtOAc→EtOAc) to afford the title compound as a white solid.

### Step 5

### (cis-1,3′)-N-Cyclopropyl-N-[3-(2-methoxyethoxy)-5-(3-methoxypropyl)benzyl]-3-oxo-3H-spiro[2-benzofuran-1,4′-piperidine]-3′-carboxamide

To a CH₂Cl₂ solution (0.05 M) of tert-butyl(cis-1,3′)-3′-({cyclopropyl[3-(2-methoxyethoxy)-5-(3-methoxypropyl)benzyl]amino}carbonyl)-3-oxo-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 95:5 (v/v) CH₂Cl₂: 2.0 M NH₃ in MeOH. Elution with the same solvent system furnished the title compound as a white solid. MS (ESI+, M+H): 523.3.

### Example 2

### (cis-1,3′)-N-Cyclopropyl-3-hydroxy-N-[3-(2-methoxyethoxy)-5-(3-methoxypropyl)benzyl]-3H-spiro[2-benzofuran-1,4′-piperidine]-3′-carboxamide

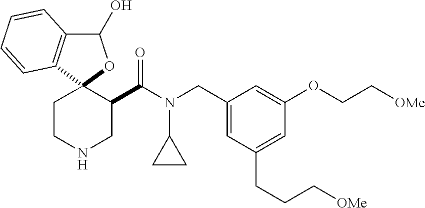

To a CH₂Cl₂ solution (0.05 M) of tert-butyl(cis-1,3′)-3′-({cyclopropyl[3-(2-methoxyethoxy)-5-(3-methoxypropyl)benzyl]amino}carbonyl)-3-hydroxy-1′H,3H-spiro[2-benzofuran-1,4′-piperidine]-1′-carboxylate (1 eq., Example 1, Step 3) was added HCl (4.0 M dioxane solution, 30 eq.). The resulting solution was stirred at RT for 3 h. Following the removal of the volatiles in vacuo, the resulting residue was directly loaded onto a SiO₂ column packed with 94:6 (v/v) CH₂Cl₂: 2.0 M NH₃ in MeOH. Elution with the same solvent system furnished the title compound as a white solid. MS (ESI+, M+H): 525.3.

### Example 3

### (cis-1,3′)-N-Cyclopropyl-N-[3-(2-methoxyethoxy)-5-(3-methoxypropyl)benzyl]-3H-spiro[2-benzofuran-1,4′-piperidine]-3′-carboxamide

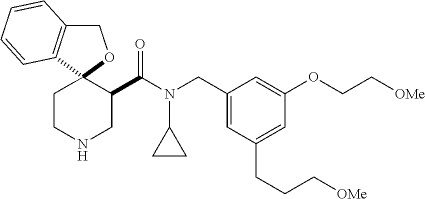

To a methanol suspension (0.1 M) of (cis-1,3′)-N-cyclopropyl-3-hydroxy-N-[3-(2-methoxyethoxy)-5-(3-methoxypropyl)benzyl]-3H-spiro[2-benzofuran-1,4′-piperidine]-3′-carboxamide (1 eq., Example 2) and palladium black (10% (w/w) over carbon, 0.1 eq.) was added trifluoroacetic acid (50 eq.). The vessel was then evacuated and back-filled with hydrogen. Under a balloon-filled hydrogen atmosphere, the resulting suspension was allowed to stir at RT for 4 days. The volatiles were then removed in vacuo and the resulting residue was partitioned between dichloromethane and 1 N aq. NaOH. The organic layer was separated, washed further with brine, dried over Na₂SO₄, filtered and the filtrate concentrated in vacuo. Purification of the crude product thus obtained by way of column chromatography (SiO₂, 95:5 (v/v) CH₂Cl₂: 2.0 M NH₃ in MeOH) afforded the title compound as a viscous oil. MS (ESI+, M+H): 509.4.

### Example 4

### (cis-1,3′)-N-[2-Chloro-5-(2-methoxyethyl)benzyl]-N-cyclopropyl-3-oxo-3H-spiro[2-benzofuran-1,4′-piperidine]-3′-carboxamide

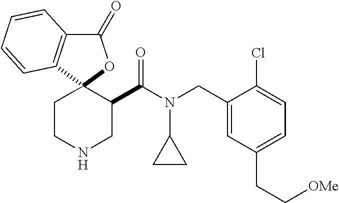

Prepared according to the procedure described in Example 1 but using N-[2-chloro-5-(2-methoxyethyl)benzyl]cyclopropanamine (1 eq., prepared according to the procedure described in WO 2007/009250 A1) in place of N-[3-(2-methoxyethoxy)-5-(3-methoxypropyl)benzyl]cyclopropanamine as the starting material in step 1. MS (ESI+, M+H): 469.2.

### Example 5

### (cis-1,3′)-N-Cyclopropyl-N-[3-(2-methoxyethoxy)-5-(3-methoxypropyl)benzyl]-3,4-dihydrospiro[isochromene-1,4′-piperidine]-3′-carboxamide

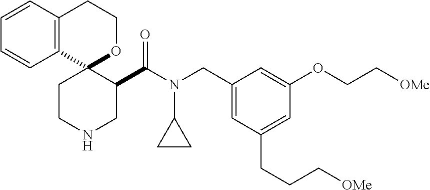

### Step 1

### tert-Butyl(cis-3,4)-3-({cyclopropyl[3-(2-methoxyethoxy)-5-(3-methoxypropyl)benzyl]amino}carbonyl)-4-hydroxy-4-{2-[2-(tetrahydro-2H-pyran-2-yloxy)ethyl]phenyl}-1-piperidinecarboxylate

To a THF solution (0.2 M) of tert-butyl 3-({cyclopropyl[3-(2-methoxyethoxy)-5-(3-methoxypropyl)benzyl]amino}carbonyl)-4-oxo-1-piperidinecarboxylate (1 eq., Example 1, Step 1) was added Grignard 1 (0.11 M THF solution, 2.3 eq.) at RT over 10 min. The resulting reaction mixture was allowed to stir at RT for 18 h before it was diluted with ether and carefully quenched with 10% aq. HCl. The aqueous layer was separated and back-extracted with ether. The combined organic extracts were washed further with brine, dried over Na₂SO₄, filtered and the filtrate concentrated in vacuo. Purification of the crude product thus obtained by way of column chromatography (SiO₂, 9:1 (v/v) Hex:EtOAc→EtOAc) afforded the title compound as a white solid.

### Step 2

### (cis-1,3′)-N-Cyclopropyl-N-[3-(2-methoxyethoxy)-5-(3-methoxypropyl)benzyl]-3,4-dihydro spiro[isochromene-1,4′-piperidine]-3′-carboxamide

To a THF solution (0.1 M) of tert-butyl(cis-3,4)-3-({cyclopropyl[3-(2-methoxyethoxy)-5-(3-methoxypropyl)benzyl]amino}carbonyl)-4-hydroxy-4-{2-[2-(tetrahydro-2H-pyran-2-yloxy)ethyl]phenyl}-1-piperidinecarboxylate (1 eq.) from the previous step was added HCl (6 M aq. solution, 30 eq.). The resulting solution was heated at reflux for 20 h. The reaction mixture was then cooled to RT, quenched with 2 N aq. NaOH and extracted with ether. The combined organic extracts were washed further with water and brine, dried over K₂CO₃, filtered and the filtrate concentrated in vacuo. Purification of the crude product thus obtained by way of column chromatography (SiO₂, 95:5 (v/v) CH₂Cl₂: 2.0 M NH₃ in MeOH) afforded the title compound as a pale yellow oil. MS (ESI+, M+H): 523.3.

### Example 6

### (cis-1,3′)-N-Cyclopropyl-5,6-difluoro-N-[3-(2-methoxyethoxy)-5-(3-methoxypropyl)benzyl]-3-oxo-3H-spiro[2-benzofuran-1,4′-piperidine]-3′-carboxamide

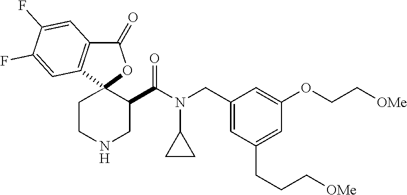

Prepared according to the procedure described in Example 1 but using Grignard 2 (1.5 eq.) in place of 2-(1,3-dioxan-2-yl)phenyl magnesium bromide as the starting material in step 2. MS (ESI+, M+H): 559.5.

### Example 7

### (cis-1,3′)-N-[2-Chloro-5-(2-methoxyethyl)benzyl]-N-cyclopropyl-5,6-difluoro-3-oxo-3H-spiro[2-benzofuran-1,4′-piperidine]-3′-carboxamide

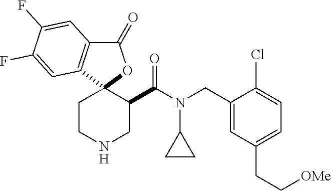

Prepared according to the procedure described in Example 4 but using Grignard 2 (1.5 eq.) in place of 2-(1,3-dioxan-2-yl)phenyl magnesium bromide as the starting material in step 2. MS (ESI+, M+H): 505.1.

### Example 8

### (cis-1,3′)-N-Cyclopropyl-5,6-difluoro-N-[3-(2-methoxyethoxy)-5-(3-methoxypropyl)benzyl]-3H-spiro[2-benzofuran-1,4′-piperidine]-3′-carboxamide

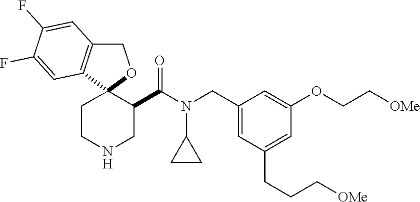

### Step 1

### tert-Butyl(cis-3,4)-3-({cyclopropyl[3-(2-methoxyethoxy)-5-(3-methoxypropyl)benzyl]amino}carbonyl)-4-[4,5-difluoro-2-(hydroxymethyl)phenyl]-4-hydroxy-1-piperidinecarboxylate

To a THF solution (0.11 M) of tert-butyl(cis-1,3′)-3′-({cyclopropyl[3-(2-methoxyethoxy)-5-(3-methoxypropyl)benzyl]amino}carbonyl)-5,6-difluoro-3-oxo-1′H,3H-spiro[2-benzofuran-1,4′-piperidine]-1′-carboxylate (1 eq., Example 6, Step 4) was added lithium borohydride (1.5 eq.). After stirring at RT for 48 h, the reaction mixture was carefully quenched with 10% aq. HCl and then extracted with ether. The combined organic extracts were washed further with 1 N aq. NaOH, water and brine, dried over Na₂SO₄, filtered and the filtrate concentrated in vacuo. Purification of the crude product thus obtained by way of column chromatography (SiO₂, 4:1 (v/v) Hex:EtOAc→EtOAc) afforded the title compound as a white solid.

### Step 2

### tert-Butyl(cis-1,3′)-3′-({cyclopropyl[3-(2-methoxyethoxy)-5-(3-methoxypropyl)benzyl]amino}carbonyl)-5,6-difluoro-1′H,3H-spiro[2-benzofuran-1,4′-piperidine]-1′-carboxylate

To a THF solution (0.05 M) of tert-butyl(cis-3,4)-3-({cyclopropyl[3-(2-methoxyethoxy)-5-(3-methoxypropyl)benzyl]amino}carbonyl)-4-[4,5-difluoro-2-(hydroxymethyl)phenyl]-4-hydroxy-1-piperidinecarboxylate (1 eq.) from the previous step and triethylamine (3 eq.) was added at 0° C. methanesulfonyl chloride (1.5 eq.). The resulting suspension was allowed to warm to RT over 3 h. The volatiles were then removed in vacuo and the residue thus obtained was partitioned between ether and 1 N aq. NaOH. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, 9:1 (v/v) Hex:EtOAc→EtOAc) afforded the title compound as a colorless oil.

### Step 3

### (cis-1,3′)-N-Cyclopropyl-5,6-difluoro-N-[3-(2-methoxyethoxy)-5-(3-methoxypropyl)benzyl]-3H-spiro[2-benzofuran-1,4′-piperidine]-3′-carboxamide

To a CH₂Cl₂ solution (0.05 M) of tert-butyl(cis-1,3′)-3′-({cyclopropyl[3-(2-methoxyethoxy)-5-(3-methoxypropyl)benzyl]amino}carbonyl)-5,6-difluoro-1′H,3H-spiro[2-benzofuran-1,4′-piperidine]-F-carboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 95:5 (v/v) CH₂Cl₂: 2.0 M NH₃ in MeOH. Elution with the same solvent system furnished the title compound as a white solid. MS (ESI+, M+H): 545.3.

### Example 9

### (cis-1,3′)-N-[2-Chloro-5-(2-methoxyethyl)benzyl]-N-cyclopropyl-5,6-difluoro-3H-spiro[2-benzofuran-1,4′-piperidine]-3′-carboxamide

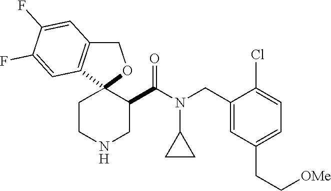

Prepared according to the procedure described in Example 8 but using tert-butyl (cis-1,3′)-3′-{[[2-chloro-5-(2-methoxyethyl)benzyl](cyclopropyl)amino]carbonyl}-5,6-difluoro-3-oxo-1′H,3H-spiro[2-benzofuran-1,4′-piperidine]-1-carboxylate (1 eq., Example 7, Step 4) in place of tert-butyl(cis-1,3′)-3′-({cyclopropyl[3-(2-methoxyethoxy)-5-(3-methoxypropyl)benzyl]amino}carbonyl)-5,6-difluoro-3-oxo-1′H,3H-spiro[2-benzofuran-1,4′-piperidine]-F-carboxylate as the starting material in step 1. MS (ESI+, M+H): 491.4.

### Example 10

### (cis-1,3′)-N-Cyclopropyl-N-[3-(2-methoxyethoxy)-5-(3-methoxypropyl)benzyl]spiro[isochromene-1,4′-piperidine]-3′-carboxamide

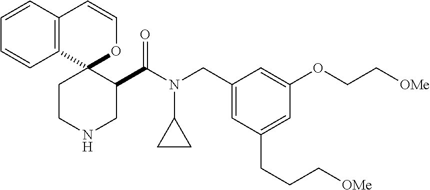

### Step 1

### tert-Butyl(cis-3,4)-3-({cyclopropyl[3-(2-methoxyethoxy)-5-(3-methoxypropyl)benzyl]amino}carbonyl)-4-[2-(1,3-dioxan-2-ylmethyl)phenyl]-4-hydroxy-1-piperidinecarboxylate

To a THF solution (0.05 M) of tert-butyl 3-({cyclopropyl[3-(2-methoxyethoxy)-5-(3-methoxypropyl)benzyl]amino}carbonyl)-4-oxo-1-piperidinecarboxylate (1 eq., Example 1, Step 1) was added Grignard 3 (1.7 eq.) at RT over a period of 10 min. The resulting dark suspension was allowed to stir at RT for another 18 h. The reaction mixture was then diluted with ether and carefully quenched with 10% aq. HCl. The aqueous layer was separated and back-extracted with ether. The combined organic extracts were washed further with brine, dried over Na₂SO₄, filtered and the filtrate concentrated in vacuo. Purification of the crude product thus obtained by way of column chromatography (SiO₂, 6:1 (v/v) Hex:EtOAc→EtOAc) afforded the title compound as a white solid.

### Step 2

### tert-Butyl(cis-1,3′)-3′-({cyclopropyl[3-(2-methoxyethoxy)-5-(3-methoxypropyl)benzyl]amino}carbonyl)-3-hydroxy-3,4-dihydro-1′H-spiro[isochromene-1,4′-piperidine]-1′-carboxylate

To a 10:1 (v/v) acetone: water solution (0.02 M) of tert-butyl(cis-3,4)-3-({cyclopropyl[3-(2-methoxyethoxy)-5-(3-methoxypropyl)benzyl]amino}carbonyl)-4-[2-(1,3-dioxan-2-ylmethyl)phenyl]-4-hydroxy-1-piperidinecarboxylate (1 eq.) from the previous step was added PPTS (0.3 eq.). The resulting solution was heated at 80° C. for 3 h. The volatiles were then removed in vacuo and the resulting residue was partitioned between ether and 1 N aq. NaOH. The aqueous wash was separated and back-extracted with ether. The combined organic extracts were washed further with water and brine, dried over Na₂SO₄ and filtered. Concentration of the filtrate thus obtained afforded the title compound as a white solid.

### Step 3

### tert-Butyl(cis-1,3′)-3′-({cyclopropyl[3-(2-methoxyethoxy)-5-(3-methoxypropyl)benzyl]amino}carbonyl)-1′H-spiro[isochromene-1,4′-piperidine]-1′-carboxylate

To a dichloromethane solution (0.06 M) of tert-butyl(cis-1,3′)-3′-({cyclopropyl[3-(2-methoxyethoxy)-5-(3-methoxypropyl)benzyl]amino}carbonyl)-3-hydroxy-3,4-dihydro-1′H-spiro[isochromene-1,4′-piperidine]-F-carboxylate (1 eq.) from the previous step and triethylamine (3 eq.) was added at 0 methanesulfonyl chloride (1.5 eq.). The resulting suspension was allowed to warm to RT over 3 h. The volatiles were then removed in vacuo and the residue thus obtained was partitioned between ether and 1 N aq. NaOH. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, 9:1 (v/v) Hex:EtOAc→EtOAc) afforded the title compound as a colorless oil.

### Step 4

### (cis-1,3′)-N-Cyclopropyl-N-[3-(2-methoxyethoxy)-5-(3-methoxypropyl)benzyl]spiro[isochromene-1,4′-piperidine]-3′-carboxamide

To a CH₂Cl₂ solution (0.05 M) of tert-butyl(cis-1,3′)-3′-({cyclopropyl[3-(2-methoxyethoxy)-5-(3-methoxypropyl)benzyl]amino}carbonyl)-1′H-spiro[isochromene-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 96:4 (v/v) CH₂Cl₂: 2.0 M NH₃ in MeOH. Elution with the same solvent system furnished the title compound as a white solid. MS (ESI+, M+H): 521.3.

### Example 11

### (cis-1,3′)-N-[2-Chloro-5-(2-methoxyethyl)benzyl]-N-cyclopropylspiro[isochromene-1,4′-piperidine]-3′-carboxamide

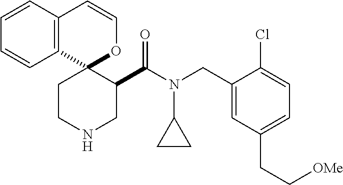

Prepared according to the procedure described in Example 10 but using tert-butyl 3-{[[2-chloro-5-(2-methoxyethyl)benzyl] (cyclopropyl)amino]carbonyl}-4-oxo-1-piperidinecarboxylate (1 eq., Example 4, Step 1) in place of tert-butyl 3-({cyclopropyl[3-(2-methoxyethoxy)-5-(3-methoxypropyl)benzyl]amino}carbonyl)-4-oxo-1-piperidinecarboxylate as the starting material in step 1. MS (ESI+, M+H): 467.2.

### Example 12

### (cis-1,3′)-N-Cyclopropyl-N-[3-(2-methoxyethoxy)-5-(3-methoxypropyl)benzyl]-3-oxo-3,4-dihydrospiro[isochromene-1,4′-piperidine]-3′-carboxamide

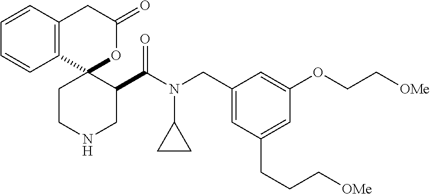

### Step 1

### tert-Butyl(cis-1,3′)-3′-({cyclopropyl[3-(2-methoxyethoxy)-5-(3-methoxypropyl)benzyl]amino}carbonyl)-3-oxo-3,4-dihydro-1′H-spiro[isochromene-1,4′-piperidine]-1′-carboxylate

To a dichloromethane suspension (0.05 M) of tert-butyl(cis-1,3′)-3′-({cyclopropyl[3-(2-methoxyethoxy)-5-(3-methoxypropyl)benzyl]amino}carbonyl)-3-hydroxy-3,4-dihydro-1′H-spiro[isochromene-1,4′-piperidine]-1′-carboxylate (1 eq., Example 10, Step 2), NMO (2.1 eq.) and 4 Å molecular sieves was added TPAP (0.1 eq.). After stirring at RT for 1 h, the reaction mixture was diluted with ether and filtered through a pad of celite. The filtrate was concentrated in vacuo and the crude product thus obtained was immediately subjected to purification by way of column chromatography (SiO₂, 9:1 (v/v) Hex:EtOAc→EtOAc) to finally the title compound as a viscous oil.

### Step 2

### (cis-1,3′)-N-Cyclopropyl-N-[3-(2-methoxyethoxy)-5-(3-methoxypropyl)benzyl]-3-oxo-3,4-dihydrospiro[isochromene-1,4′-piperidine]-3′-carboxamide

To a CH₂Cl₂ solution (0.05 M) of tert-butyl(cis-1,3′)-3′-({cyclopropyl[3-(2-methoxyethoxy)-5-(3-methoxypropyl)benzyl]amino}carbonyl)-3-oxo-3,4-dihydro-1′H-spiro[isochromene-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 96:4 (v/v) CH₂Cl₂: 2.0 M NH₃ in MeOH. Elution with the same solvent system furnished the title compound as a colorless oil. MS (ESI+, M+H): 537.3.

### Example 13

### (cis-1,3′)-N-[2-Chloro-5-(2-methoxyethyl)benzyl]-N-cyclopropyl-5-methyl-6-oxo-5,6-dihydro-3H-spiro[furo[3,4-c]pyridine-1,4′-piperidine]-3′-carboxamide

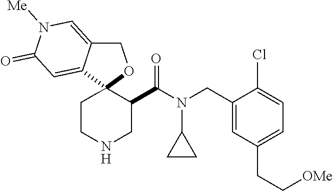

### Step 1

### (4-Iodo-6-methoxy-3-pyridinyl)methanol

To a THF solution (0.26 M) of trimethylethylenediamine (1.2 eq.) was added nBuLi (2.5 M cyclohexane solution, 1.1 eq.) dropwise at −78° C. After 15 min, 6-methoxynicotinaldehyde (1 eq.) was then added in one rapid portion. The resulting solution was stirred at −78° C. for another 15 min before nBuLi (2.5 M cyclohexane solution, 2 eq.) was added dropwise over 10 min. The reaction mixture was then allowed to warm slowly to −40° C. and allowed to stir at −40° C. for another 2 h. The reaction mixture was re-cooled to −78° C. before freshly re-crystallized 1,2-diiodoethane (2.1 eq.) was added. Finally, the cooling bath was removed and the resulting red suspension was allowed to stir at RT for 16 h. The reaction mixture was then diluted with ether and quenched with sat. aq. NaHSO₃. The organic layer was separated and washed further with 1 N aq. NaOH, water and brine. The organic extract was then dried over Na₂SO₄, filtered and the filtrate concentrated in vacuo. Purification of the crude product thus obtained by way of column chromatography (SiO₂, 9:1 (v/v) Hex:EtOAc→3:7 (v/v) Hex:EtOAc) afforded the title compound as a white solid.

### Step 2

### 4-Iodo-2-methoxy-5-{[(triisopropylsilyl)oxy]methyl}pyridine

To a DMF solution (0.2 M) of (4-iodo-6-methoxy-3-pyridinyl)methanol (1 eq.) from the previous step and imidazole (1.5 eq.) was added chlorotriisopropylsilane (1.2 eq.). The resulting solution was allowed to stir at RT for 24 h before it was diluted with ether and washed with water and brine. The organic extract was dried over Na₂SO₄, filtered and the filtrate concentrated in vacuo. Purification of the crude product thus obtained by way of column chromatography (SiO₂, 9:1 (v/v) Hex:EtOAc→3:7 (v/v) Hex:EtOAc) afforded the title compound as a colorless oil.

### Step 3

### tert-Butyl(cis-3,4)-3-{[[2-chloro-5-(2-methoxyethyl)benzyl](cyclopropyl)amino]carbonyl}-4-hydroxy-4-(2-methoxy-5-{[(triisopropylsilyl)oxy]methyl}-4-pyridinyl)-1-piperidinecarboxylate

To a THF solution (0.06 M) of 4-iodo-2-methoxy-5-{[(triisopropylsilyl)oxy]methyl}pyridine (1.8 eq.) from the previous step was added isopropylmagnesium chloride (2 M in THF, 1.9 eq.). The resulting solution was stirred at RT for 30 min before tert-butyl 3-{[[2-chloro-5-(2-methoxyethyl)benzyl](cyclopropyl)amino]carbonyl}-4-oxo-1-piperidinecarboxylate (1 eq., Example 4, Step 1) was added in one rapid portion. After stirring at RT for 18 h, the reaction mixture was diluted with ether and carefully quenched with 1 N aq. NaOH. The aqueous layer was separated and back-extracted with ether. The combined organic extracts were washed further with brine, dried over Na₂SO₄, filtered and the filtrate concentrated in vacuo. Purification of the crude product thus obtained by way of column chromatography (SiO₂, 9:1 (v/v) Hex:EtOAc→EtOAc) afforded the title compound as a colorless oil.

### Step 4

### tert-Butyl(cis-3,4)-3-{[[2-chloro-5-(2-methoxyethyl)benzyl] (cyclopropyl)amino]carbonyl}-4-hydroxy-4-[5-(hydroxymethyl)-2-methoxy-4-pyridinyl]-1-piperidinecarboxylate

To a THF solution (0.07 M) of tert-butyl(cis-3,4)-3-{[[2-chloro-5-(2-methoxyethyl)benzyl](cyclopropyl)amino]carbonyl}-4-hydroxy-4-(2-methoxy-5-{[(triisopropylsilyl)oxy]methyl}-4-pyridinyl)-1-piperidinecarboxylate (1 eq.) from the previous step was added TBAF (1 M THF solution, 1.5 eq.). After stirring at RT for 4 h, the crude reaction mixture was diluted with EtOAc and washed with water and brine. The organic extract was then dried over Na₂SO₄, filtered and the filtrate concentrated in vacuo. Purification of the crude product thus obtained by way of column chromatography (SiO₂, 6:1 (v/v) Hex:EtOAc→EtOAc) afforded the title compound as a white solid.

### Step 5

### tert-Butyl(cis-1,3′)-3′-{[[2-chloro-5-(2-methoxyethyl)benzyl] (cyclopropyl)amino]carbonyl}-6-methoxy-1′H,3H-spiro[furo[3,4-c]pyridine-1,4′-piperidine]-1′-carboxylate

To a THF solution (0.11 M) of tert-butyl(cis-3,4)-3-{[[2-chloro-5-(2-methoxyethyl)benzyl](cyclopropyl)amino]carbonyl}-4-hydroxy-4-[5-(hydroxymethyl)-2-methoxy-4-pyridinyl]-1-piperidinecarboxylate (1 eq.) from the previous step and triethylamine (3.2 eq.) was added at 0 methanesulfonyl chloride (1.4 eq.). The resulting suspension was allowed to warm to RT over 16 h. The volatiles were then removed in vacuo and the residue thus obtained was partitioned between ether and 1 N aq. NaOH. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, 8:1 (v/v) Hex:EtOAc→EtOAc) afforded the title compound as a colorless oil.

### Step 6

### tert-Butyl(cis-1,3′)-3′-{[[2-chloro-5-(2-methoxyethyl)benzyl] (cyclopropyl)amino]carbonyl}-5-methyl-6-oxo-5,6-dihydro-1′H,3H-spiro[furo[3,4-c]pyridine-1,4′-piperidine]-1′-carboxylate

In an acetonitrile solution (0.19 M) of tert-butyl(cis-1,3′)-3′-{[[2-chloro-5-(2-methoxyethyl)benzyl](cyclopropyl)amino]carbonyl}-6-methoxy-1′H,3H-spiro[furo[3,4-c]pyridine-1,4′-piperidine]-1′-carboxylate (1 eq.) from the previous step was added sodium iodide (3 eq.) and iodomethane (6 eq.). The reaction vessel was tightly sealed before the reaction mixture was heated at 45° C. for 3 days. The volatiles were then removed in vacuo and the resulting residue was partitioned between EtOAc and 10% aq. HCl. The aqueous layer was separated and back-extracted with EtOAc. The combined organic extracts were washed further with 1 N aq. NaOH, water and brine, dried over Na₂SO₄ and filtered. Concentration of the filtrate thus obtained in vacuo afforded the title compound as a white solid.

### Step 7

### (cis-1,3′)-N-[2-Chloro-5-(2-methoxyethyl)benzyl]-N-cyclopropyl-5-methyl-6-oxo-5,6-dihydro-3H-spiro[furo[3,4-c]pyridine-1,4′-piperidine]-3′-carboxamide

To a CH₂Cl₂ solution (0.05 M) of tert-butyl(cis-1,3′)-3′-{[[2-chloro-5-(2-methoxyethyl)benzyl](cyclopropyl)amino]carbonyl}-5-methyl-6-oxo-5,6-dihydro-1′H,3H-spiro[furo[3,4-c]pyridine-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 93:7 (v/v) CH₂Cl₂: 2.0 M NH₃ in MeOH. Elution with the same solvent system furnished the title compound as a white foam. MS (ESI+, M+H): 486.1.

### Example 14

### (cis-1,3′)-N-[2-Chloro-5-(2-methoxyethyl)benzyl]-N-cyclopropyl-6-methoxy-3H-spiro[furo[3,4-c]pyridine-1,4′-piperidine]-3′-carboxamide

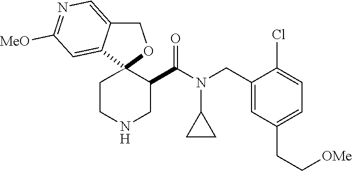

To a CH₂Cl₂ solution (0.05 M) of tert-butyl(cis-1,3′)-3′-{[[2-chloro-5-(2-methoxyethyl)benzyl] (cyclopropyl)amino]carbonyl}-6-methoxy-1′H,3H-spiro[furo[3,4-c]pyridine-1,4′-piperidine]-1′-carboxylate (1 eq., Example 13, Step 5) was added HCl (4.0 M dioxane solution, 30 eq.). The resulting solution was stirred at RT for 3 h. Following the removal of the volatiles in vacuo, the resulting residue was directly loaded onto a SiO₂ column packed with 95:5 (v/v) CH₂Cl₂: 2.0 M NH₃ in MeOH. Elution with the same solvent system furnished the title compound as a viscous oil. MS (ESI+, M+H): 486.1.

### Example 15

### (cis-1,3′)-N-Benzyl-N-cyclopropyl-5,6-difluoro-3H-spiro[2-benzofuran-1,4′-piperidine]-3-carboxamide

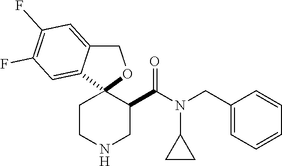

### Step 1

### tert-Butyl(cis-1,3′)-3′-{[benzyl(cyclopropyl)amino]carbonyl}-5,6-difluoro-1′H,3H-spiro[2-benzofuran-1,4′-piperidine]-1′-carboxylate

To a DMF solution (0.1 M) of Intermediate 1 (1 eq.) was added sodium hydride

(60% dispersion in oil, 1.5 eq.) and benzyl bromide (1.5 eq.). The resulting suspension was stirred at 60° C. for 2 h. After cooling to RT, the reaction mixture was carefully quenched with sat. aq. NH₄Cl and then extracted with EtOAc. The combined organic extracts were dried over Na₂SO₄, filtered and the filtrate concentrated in vacuo. Purification of the crude product thus obtained by way of column chromatography (SiO₂, 95:5 (v/v) Hex:EtOAc→EtOAc) afforded the title compound as a white solid.

### Step 2

### (cis-1,3′)-N-Benzyl-N-cyclopropyl-5,6-difluoro-3H-spiro[2-benzofuran-1,4′-piperidine]-3′-carboxamide

To a CH₂Cl₂ solution (0.1 M) of tert-butyl(cis-1,3′)-3′-{[benzyl(cyclopropyl)amino]carbonyl}-5,6-difluoro-1′H,3H-spiro[2-benzofuran-1,4′-piperidine]-F-carboxylate (1 eq.) from the previous step was added HCl (4.0 M dioxane solution, 30 eq.). The resulting solution was stirred at RT for 3 h. Following the removal of the volatiles in vacuo, the resulting residue was partitioned between EtOAc and 1 N aq. NaOH. The aqueous layer was separated and back-extracted with EtOAc. The combined organic extracts were then dried over Na₂SO₄, filtered and the filtrate concentrated in vacuo. Purification of the crude product thus obtained by way of column chromatography (SiO₂, 95:5 (v/v) CH₂Cl₂: 2.0 M NH₃ in MeOH→90:10 (v/v) CH₂Cl₂: 2.0 M NH₃ in MeOH) afforded the title compound as a white solid. MS (ESI+, M+H): 399.2.

### Example 16

### (cis-1,3′)-N-Cyclopropyl-N-(2,3-dichlorobenzyl)-5,6-difluoro-3H-spiro[2-benzofuran-1,4′-piperidine]-3′-carboxamide

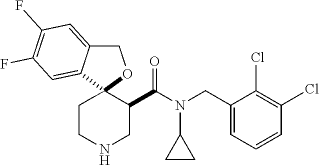

Prepared according to the procedure described in Example 15 but using 2,3-dichlorobenzyl bromide (1.5 eq.) in place of benzyl bromide as the starting material in step 1. MS (ESI+, M+H): 467.2.

### Example 17

### (cis-1,3′)-N-Cyclopropyl-N-(2,3-dimethylbenzyl)-5,6-difluoro-3H-spiro[2-benzofuran-1,4′-piperidine]-3′-carboxamide

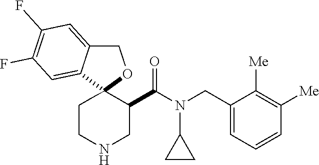

Prepared according to the procedure described in Example 15 but using Alkylation Reagent 1 (1.5 eq.) in place of benzyl bromide as the starting material in step 1. MS (ESI+, M+H): 427.3. ¹H NMR (500 MHz, CDCl₃): δ (ppm) 7.00 (d, J=7.8 Hz, 2H); 6.91-6.81 (m, 2H); 6.19 (d, J=7.2 Hz, 1H); 5.13 (d, J=12.2 Hz, 1H); 4.96 (d, J=12.2 Hz, 1H); 4.85 (d, J=15.4 Hz, 1H); 4.02 (d, J=15.4 Hz, 1H); 3.69 (d, J=9.8 Hz, 1H); 3.44 (t, J=11.4 Hz, 1H); 3.24 (br, 1H); 3.12-2.99 (m, 2H); 2.44 (s, 1H); 2.25 (s, 3H); 2.06 (s, 3H); 1.87 (s, 2H); 1.78 (br, 1H); 1.02 (br, 1H); 0.80 (s, 2H); 0.72 (s, 1H). Human Renin IC₅₀ (buffer): 0.1 nM. Human Renin IC₅₀ (plasma): 0.5 nM.

### Example 18

### (cis-1,3′)-N-Cyclopropyl-5,6-difluoro-N-(4-quinolinylmethyl)-3H-spiro[2-benzofuran-1,4′-piperidine]-3′-carboxamide

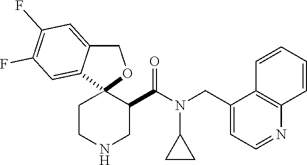

Prepared according to the procedure described in Example 15 but using Alkylation Reagent 2 (1.5 eq.) in place of benzyl bromide as the starting material in step 1. MS (ESI+, M+H): 450.3. ¹H NMR (500 MHz, CDCl₃): δ (ppm) 8.69 (d, J=4.4 Hz, 1H); 8.13 (d, J=8.5 Hz, 1H); 7.97 (d, J=8.4 Hz, 1H); 7.75-7.70 (m, 1H); 7.57-7.52 (m, 1H); 6.97 (dd, J=9.3, 6.8 Hz, 1H); 6.64-6.57 (m, 2H); 5.29 (d, J=15.6 Hz, 1H); 4.95 (d, J=12.2 Hz, 1H); 4.88 (d, J=12.2 Hz, 1H); 4.47 (d, J=15.6 Hz, 1H); 3.70 (dd, J=10.1, 3.8 Hz, 1H); 3.44 (dd, J=12.4, 10.2 Hz, 1H); 3.28-3.17 (m, 1H); 3.10 (dd, J=12.5, 3.7 Hz, 1H); 3.03 (dt, J=12.5, 4.0 Hz, 1H); 2.55-2.49 (m, 1H); 1.90-1.83 (m, 2H); 1.73 (br, 2H); 1.16-1.08 (m, 1H); 0.97-0.77 (m, 3H). Human Renin IC₅₀ (buffer): 3.1 nM. Human Renin IC₅₀ (plasma): 6.4 nM.

### Example 19

### (cis-1,3′)-N-[3-Bromo-5-(3-hydroxypropyl)-4-methylbenzyl]-N-cyclopropyl-5,6-difluoro-3H-spiro[2-benzofuran-1,4′-piperidine]-3′-carboxamide

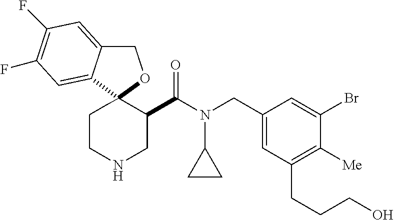

Prepared according to the procedure described in Example 15 but using Alkylation Reagent 3 (1.5 eq.) in place of benzyl bromide as the starting material in step 1. MS (ESI+, M+H): 549.2.

### Example 20

### (cis-1,3′)-N-[3-Bromo-5-(3-methoxypropyl)-4-methylbenzyl]-N-cyclopropyl-5,6-difluoro-3H-spiro[2-benzofuran-1,4′-piperidine]-3′-carboxamide

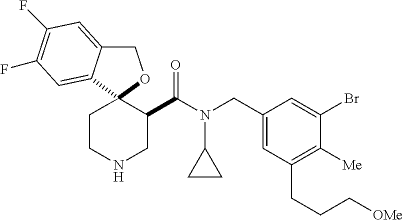

Prepared according to the procedure described in Example 15 but using Alkylation Reagent 4 (1.5 eq.) in place of benzyl bromide as the starting material in step 1. MS (ESI+, M+H): 563.2.

### Example 21

### (cis-1,3′)-N-Cyclopropyl-5,6-difluoro-N-(1-naphthylmethyl)-3H-spiro[2-benzofuran-1,4′-piperidine]-3′-carboxamide

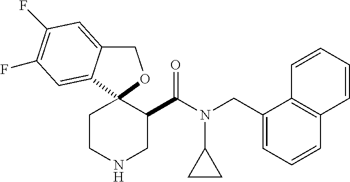

Prepared according to the procedure described in Example 15 but using 1-(bromomethyl)naphthalene (1.5 eq.) in place of benzyl bromide as the starting material in step 1. MS (ESI+, M+H): 449.3. ¹H NMR (500 MHz, CDCl₃): δ (ppm) 8.05 (dd, J=7.6, 2.1 Hz, 1H), 7.84 (dd, J=7.2, 2.4 Hz, 1H), 7.73 (d, J=8.2 Hz, 1H), 7.49-7.44 (m, 2H), 6.90-6.80 (m, 2H), 6.35-6.31 (m, 1H), 5.30 (d, J=14.8 Hz, 1H), 4.93 (d, J=12.1 Hz, 1H), 4.79 (d, J=12.0 Hz, 1H), 4.38 (d, J=14.8 Hz, 1H), 3.63 (dd, J=10.3, 3.7 Hz, 1H), 3.43 (dd, J=12.4, 10.6 Hz, 1H), 3.25-3.18 (m, 1H), 3.06-2.96 (m, 2H), 2.36-2.33 (m, 1H), 1.81-1.77 (m, 2H), 1.07-1.05 (m, 1H), 0.92-0.79 (m, 3H). Human Renin IC₅₀ (buffer): 0.2 nM. Human Renin IC₅₀ (plasma): 0.8 nM.

### Example 22

### (cis-1,3′)-N-Cyclopropyl-N-(3,4-dichlorobenzyl)-5,6-difluoro-3H-spiro[2-benzofuran-1,4′-piperidine]-3′-carboxamide

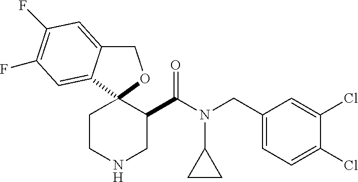

Prepared according to the procedure described in Example 15 but using 1,2-dichloro-4-(chloromethyl)benzene (1.5 eq.) in place of benzyl bromide as the starting material in step 1. MS (ESI+, M+H): 467.2.

### Example 23

### (cis-1,3′)-N-(5-Bromo-2-chlorobenzyl)-N-cyclopropyl-5,6-difluoro-3H-spiro[2-benzofuran-1,4′-piperidine]-3′-carboxamide

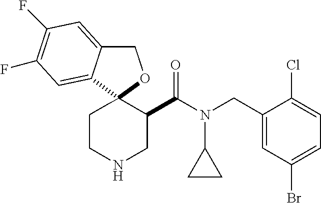

Prepared according to the procedure described in Example 15 but using Alkylation Reagent 5 (1.5 eq.) in place of benzyl bromide as the starting material in step 1. MS (ESI+, M+H): 513.2.

### Example 24

### (cis-1,3′)-N-Cyclopropyl-N-[3-(difluoromethoxy)benzyl]-5,6-difluoro-3H-spiro[2-benzofuran-1,4′-piperidine]-3′-carboxamide

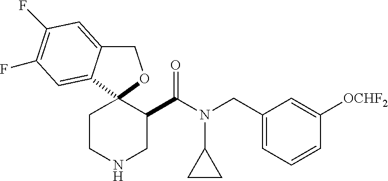

Prepared according to the procedure described in Example 15 but using 1-(bromomethyl)-3-(difluoromethoxy)benzene (1.5 eq.) in place of benzyl bromide as the starting material in step 1. MS (ESI+, M+H): 465.2.

### Example 25

### (cis-1,3′)-N-Cyclopropyl-5,6-difluoro-N-[(2-methyl-1-napthyl)methyl]-3H-spiro[2-benzofuran-1,4′-piperidine]-3′-carboxamide

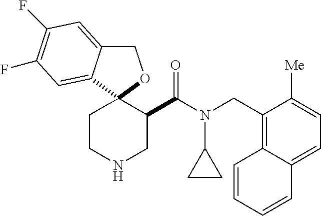

Prepared according to the procedure described in Example 15 but using 1-(bromomethyl)-2-methylnaphthalene (1.5 eq.) in place of benzyl bromide as the starting material in step 1. MS (ESI+, M+H): 463.3.

### Example 26

### (cis-1,3′)-N-Cyclopropyl-5,6-difluoro-N-(8-quinolinylmethyl)-3H-spiro[2-benzofuran-1,4′-piperidine]-3′-carboxamide

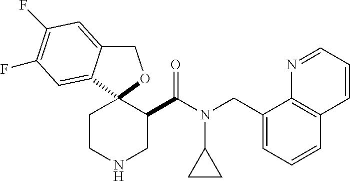

Prepared according to the procedure described in Example 15 but using 8-bromomethyl(quinoline) (1.5 eq.) in place of benzyl bromide as the starting material in step 1. MS (ESI+, M+H): 450.1. ¹H NMR (500 MHz, CDCl₃): δ (ppm) 8.85 (dd, J=4.2, 1.7 Hz, 1H); 8.14 (dd, J=8.3, 1.7 Hz, 1H); 7.68 (d, J=8.2 Hz, 1H); 7.41 (dd, J=8.3, 4.2 Hz, 1H); 7.34-7.27 (m, 1H); 7.01-6.93 (m, 1H); 6.85 (d, J=7.1 Hz, 1H); 6.73-6.65 (m, 1H); 5.23 (d, J=15.4 Hz, 1H); 5.05-4.86 (m, 3H); 3.72 (dd, J=10.6, 3.7 Hz, 1H); 3.49 (t, J=11.5 Hz, 1H); 3.24 (td, J=11.9, 3.2 Hz, 1H); 3.13 (dd, J=12.5, 3.7 Hz, 1H); 3.08-3.01 (m, 1H); 2.63-2.57 (m, 1H); 1.94-1.79 (m, 2H); 1.73 (br, 2H); 1.07-1.01 (m, 1H); 0.85-0.76 (m, 3H). Human Renin IC₅₀ (buffer): 0.7 nM. Human Renin IC₅₀ (plasma): 2.5 nM

### Example 27

### (cis-1,3′)-N-Cyclopropyl-N-(2,3-dimethoxybenzyl)-5,6-difluoro-3H-spiro[2-benzofuran-1,4′-piperidine]-3′-carboxamide

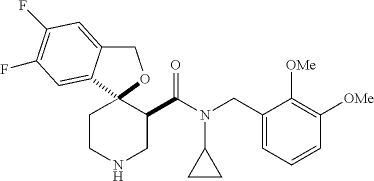

Prepared according to the procedure described in Example 15 but using Alkylation Reagent 6 (1.5 eq.) in place of benzyl bromide as the starting material in step 1. MS (ESI+, M+H): 459.3.

### Example 28

### (cis-1,3′)-N-Cyclopropyl-5,6-difluoro-N-[(2-methyl-3-pyridinyl)methyl]-3H-spiro[2-benzofuran-1,4′-piperidine]-3′-carboxamide

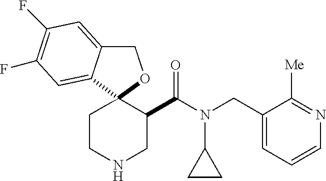

Prepared according to the procedure described in Example 15 but using Alkylation Reagent 7 (1.5 eq.) in place of benzyl bromide as the starting material in step 1. MS (ESI+, M+H): 414.1.

### Example 29

### (cis-1,3′)-N-Cyclopropyl-5,6-difluoro-N-[2-methoxy-5-(2-methoxyethyl)benzyl]-3H-spiro[2-benzofuran-1,4′-piperidine]-3′-carboxamide

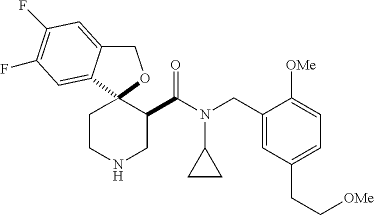

Prepared according to the procedure described in Example 15 but using Alkylation Reagent 8 (1.5 eq.) in place of benzyl bromide as the starting material in step 1. MS (ESI+, M+H): 487.2.

### Example 30

### (cis-1,3′)-N-Cyclopropyl-5,6-difluoro-N-(1 isoquinolinylmethyl)-3H-spiro[2-benzofuran-1,4′-piperidine]-3′-carboxamide

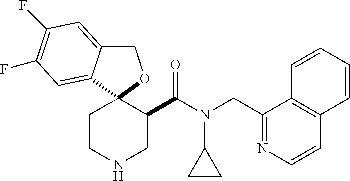

Prepared according to the procedure described in Example 15 but using 1-(bromomethyl)isoquinoline (1.5 eq.) in place of benzyl bromide as the starting material in step 1. MS (ESI+, M+H): 450.1.

### Example 31

### (cis-1,3′)-N-[(3-Bromo-1-naphthyl)methyl]-N-cyclopropyl-5,6-difluoro-3H-spiro[2-benzofuran-1,4′-piperidine]-3′-carboxamide

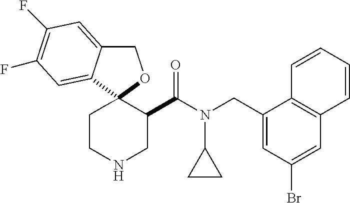

Prepared according to the procedure described in Example 15 but using Alkylation Reagent 9 (1.5 eq.) in place of benzyl bromide as the starting material in step 1. MS (ESI+, M+H): 527.1

### Example 32

### (cis-1,3′)-N-Cyclopropyl-5,6-difluoro-N-{[3-(3-methoxypropyl)-1-naphthyl]methyl}-3H-spiro[2-benzofuran-1,4′-piperidine]-3′-carboxamide

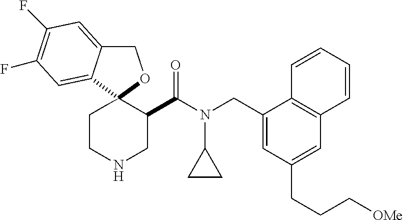

### Step 1

### tert-Butyl(cis-1,3′)-3′-{[cyclopropyl({3-[(1E)-3-methoxy-1-propen-1-yl]-1-naphthyl}methyl)amino]carbonyl}-5,6-difluoro-1′H,3H-spiro[2-benzofuran-1,4′-piperidine]-1′-carboxylate

To a DMF solution (0.1 M) of tert-butyl(cis-1,3′)-3′-{[[(3-bromo-1-naphthyl)methyl](cyclopropyl)amino]carbonyl}-5,6-difluoro-1′H,3H-spiro[2-benzofuran-1,4′-piperidine]-1′-carboxylate (1 eq., Example 31, Step 1) and 4,4,5,5-tetramethyl-2-[(1E)-3-(methyloxy)-1-propen-1-yl]-1,3,2-dioxaborolane (1.5 eq.) was added trans-bis(triphenylphosphine) palladium(II) bromide (0.1 eq.). The vessel was repeatedly evacuated and back-filled with nitrogen. Finally, Na₂CO₃ (2 M aq. solution, 3 eq.) was added and the resulting mixture was heated at 100° C. for 3 h. The now black suspension was cooled to RT, diluted with water and extracted with EtOAc. The combined organic extracts were washed further with brine, dried over Na₂SO₄, filtered and the filtrate concentrated in vacuo. Purification of the crude product by way of flash chromatography (SiO₂, 9:1 (v/v) Hex:EtOAc→EtOAc) afforded the title compound.

### Step 2

### tert-Butyl(cis-1,3′)-3′-[(cyclopropyl{[3-(3-methoxypropyl)-1-naphthyl]methyl}amino)carbonyl]-5,6-difluoro-1′H,3H-spiro[2-benzofuran-1,4′-piperidine]-1′-carboxylate

To an ethyl acetate solution (0.02 M) of tert-butyl(cis-1,3′)-3′-{[cyclopropyl({3-[(1E)-3-methoxy-1-propen-1-yl]-1-naphthyl}methyl)amino]carbonyl}-5,6-difluoro-1′H,3H-spiro[2-benzofuran-1,4′-piperidine]-1′-carboxylate (1 eq.) from the previous step was added palladium black (10% (w/w) over activated carbon, 0.3 eq.). The vessel was evacuated and back-filled with hydrogen. Under a balloon-maintained hydrogen atmosphere, the reaction suspension was stirred at RT for 3 h. The reaction mixture was then diluted with dichloromethane and filtered through a pad of celite. Concentration of the filtrate in vacuo afforded the title compound as a white foam.

### Step 3

### (cis-1,3′)-N-Cyclopropyl-5,6-difluoro-N-{[3-(3-methoxypropyl)-1-naphthyl]methyl}-3H-spiro[2-benzofuran-1,4′-piperidine]-3′-carboxamide

To a CH₂Cl₂ solution (0.1 M) of tert-butyl(cis-1,3′)-3′-[(cyclopropyl{[3-(3-methoxypropyl)-1-naphthyl]methyl}amino)carbonyl]-5,6-difluoro-1′H,3H-spiro[2-benzofuran-1,4′-piperidine]-F-carboxylate (1 eq.) from the previous step was added HCl (4.0 M dioxane solution, 20 eq.). The resulting solution was stirred at RT for 3 h. Following the removal of the volatiles in vacuo, the resulting residue was directly loaded onto a SiO₂ column packed with 95:5 (v/v) CH₂Cl₂: 2.0 M NH₃ in MeOH. Elution with the same solvent system furnished the title compound as a white foam. MS (ESI+, M+H): 521.3. ¹H NMR (500 MHz, CDCl₃): δ (ppm) 8.09 (d, J=8.4 Hz, 1H), 7.80 (d, J=8.1 Hz, 1H), 7.56 (s, 1H), 7.51-7.41 (m, 2H), 7.16 (s, 1H), 6.79 (t, J=8.0 Hz, 1H), 6.17 (t, J=8.1 Hz, 1H), 5.28 (d, J=14.6 Hz, 1H), 4.94 (d, J=12.1 Hz, 1H), 4.79 (d, J=12.1 Hz, 1H), 4.40 (d, J=14.6 Hz, 1H), 3.62 (dd, J=10.1, 3.8 Hz, 1H), 3.48-3.36 (m, 6H), 3.23-3.17 (m, 1H), 3.07 (dd, J=12.6, 3.7 Hz, 1H), 3.03-2.97 (m, 1H), 2.82-2.75 (m, 2H), 2.33-2.29 (m, 1H), 2.00-1.93 (m, 2H), 1.81-1.76 (m, 2H), 1.10-1.05 (m, 1H), 1.01-0.96 (m, 1H), 0.92-0.82 (m, 2H). Human Renin IC₅₀ (buffer): 0.1 nM. Human Renin IC₅₀ (plasma): 5.1 nM.

### Example 33

### (cis-1,3′)-N-Cyclopropyl-5,6-difluoro-N-[(6-methyl-2-pyridinyl)methyl]-3H-spiro[2-benzofuran-1,4′-piperidine]-3′-carboxamide

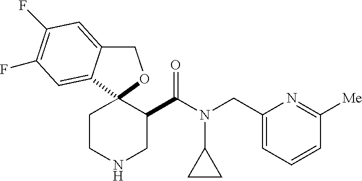

Prepared according to the procedure described in Example 15 but using 2-(bromomethyl)-6-methylpyridine (1.5 eq.) in place of benzyl bromide as the starting material in step 1. MS (ESI+, M+H): 414.4.

### Example 34

### (cis-1,3′)-N-Cyclopropyl-5,6-difluoro-N-[(3-methyl-2-pyridinyl)methyl]-3H-spiro[2-benzofuran-1,4′-piperidine]-3′-carboxamide

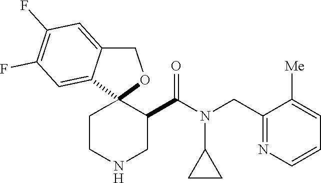

Prepared according to the procedure described in Example 15 but using 2-chloromethyl-3-methylpyridine (1.5 eq.) in place of benzyl bromide as the starting material in step 1. MS (ESI+, M+H): 414.4.

### Example 35

### (cis-1,3′)-N-(2-Chlorobenzyl)-N-cyclopropyl-5,6-difluoro-3H-spiro[2-benzofuran-1,4′-piperidine]-3′-carboxamide

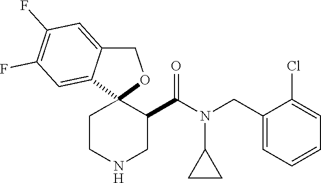

Prepared according to the procedure described in Example 15 but using 1-chloro-2-(chloromethyl)benzene (1.5 eq.) in place of benzyl bromide as the starting material in step 1. MS (ESI+, M+H): 433.1

### Example 36

### (cis-1,3′)-N-(2-Bromobenzyl)-N-cyclopropyl-5,6-difluoro-3H-spiro[2-benzofuran-1,4′-piperidine]-3′-carboxamide

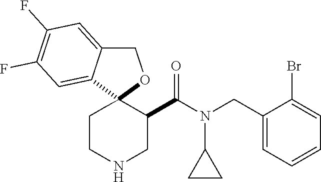

Prepared according to the procedure described in Example 15 but using 1-bromo-2-(bromomethyl)benzene (1.5 eq.) in place of benzyl bromide as the starting material in step 1. MS (ESI+, M+H): 477.2.

### Example 37

### (cis-1,3′)-N-Cyclopropyl-5,6-difluoro-N-[2-(trifluoromethyl)benzyl]-3H-spiro[2-benzofuran-1,4′-piperidine]-3′-carboxamide

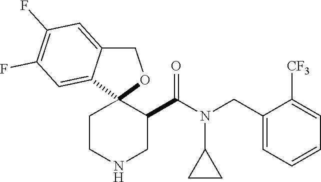

Prepared according to the procedure described in Example 15 but using 1-(bromomethyl)-2-(trifluoromethyl)benzene (1.5 eq.) in place of benzyl bromide as the starting material in step 1. MS (ESI+, M+H): 467.2.

### Example 38

### (cis-1,3′)-N-Cyclopropyl-5,6-difluoro-N-[2-methyl-3-(trifluoromethyl)benzyl]-3H-spiro[2-benzofuran-1,4′-piperidine]-3′-carboxamide

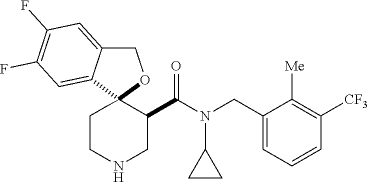

Prepared according to the procedure described in Example 15 but using 1-(bromomethyl)-2-methyl-3-(trifluoromethyl)benzene (1.5 eq.) in place of benzyl bromide as the starting material in step 1. MS (ESI+, M+H): 481.1. ¹H NMR (500 MHz, CDCl₃): δ (ppm) 8.57 (s, 1H), 7.50 (d, J=8.0 Hz, 1H), 7.21 (t, J=7.9 Hz, 1H), 7.03 (t, J=7.9 Hz, 1H), 6.93 (t, J=7.9 Hz, 1H); 6.46 (d, J=7.9 Hz, 1H), 5.17-4.86 (m, 3H), 4.24 (dd, J=11.1, 3.6 Hz, 1H), 3.98 (d, J=14.5, 1H), 3.70-3.60 (m, 1H), 3.45-3.28 (m, 3H), 2.52-2.37 (m, 2H), 2.30 (s, 3H), 1.93-1.87 (m, 1H), 1.25-1.17 (m, 1H), 0.92-0.68 (m, 3H). Human Renin IC₅₀ (buffer): 0.3 nM. Human Renin IC₅₀ (plasma): 5.4 nM.

### Example 39

### (cis-1,3′)-N-[(4-Bromo-1-naphthyl)methyl]-N-cyclopropyl-5,6-difluoro-3H-spiro[2-benzofuran-1,4′-piperidine]-3′-carboxamide

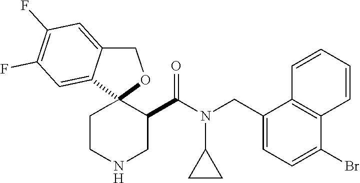

Prepared according to the procedure described in Example 15 but using 1-bromo-4-(bromomethyl)naphthalene (1.5 eq.) in place of benzyl bromide as the starting material in step 1. MS (ESI+, M+H): 527.1

### Example 40

### (cis-1,3′)-N-[3-Chloro-5-(trifluoromethyl)benzyl]-N-cyclopropyl-5,6-difluoro-3H-spiro[2-benzofuran-1,4′-piperidine]-3′-carboxamide

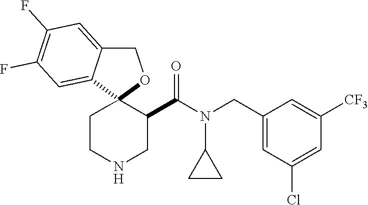

Prepared according to the procedure described in Example 15 but using 3-chloro-5-(trifluoromethyl)benzyl bromide (1.5 eq.) in place of benzyl bromide as the starting material in step 1. MS (ESI+, M+H): 501.2.

### Example 41

### (cis-1,3′)-N-(2-Chloro-4-fluorobenzyl)-N-cyclopropyl-5,6-difluoro-3H-spiro[2-benzofuran-1,4′-piperidine]-3′-carboxamide

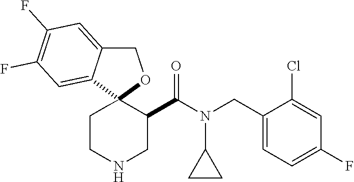

Prepared according to the procedure described in Example 15 but using 2-chloro-4-fluorobenzyl bromide (1.5 eq.) in place of benzyl bromide as the starting material in step 1. MS (ESI+, M+H): 451.3.

### Example 42

### Methyl 3-chloro-4-[(cyclopropyl{[(cis-1,3′)-5,6-difluoro-3H-spiro[2-benzofuran-1,4′-piperidin]-3′-yl]carbonyl}amino)methyl]benzoate

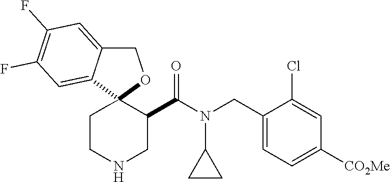

Prepared according to the procedure described in Example 15 but using methyl 4-(bromomethyl)-3-chlorobenzoate (1.5 eq.) in place of benzyl bromide as the starting material in step 1. MS (ESI+, M+H): 471.2.

### Example 43

### (cis-1,3′)-N-[2-Chloro-3-(trifluoromethyl)benzyl]-N-cyclopropyl-5,6-difluoro-3H-spiro[2-benzofuran-1,4′-piperidine]-3′-carboxamide

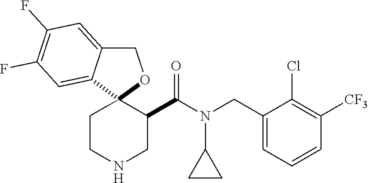

Prepared according to the procedure described in Example 15 but using 2-chloro-3-(trifluoromethyl)benzyl bromide (1.5 eq.) in place of benzyl bromide as the starting material in step 1. MS (ESI+, M+H): 501.2

### Example 44

### (cis-1,3′)-N-Cyclopropyl-5,6-difluoro-N-[2-methyl-5-(trifluoromethyl)benzyl]-3H-spiro[2-benzofuran-1,4′-piperidine]-3′-carboxamide

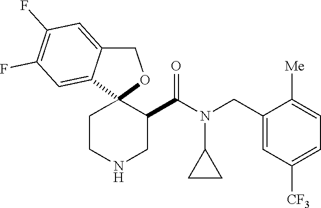

Prepared according to the procedure described in Example 15 but using 2-methyl-5-(trifluoromethyl)benzyl bromide (1.5 eq.) in place of benzyl bromide as the starting material in step 1. MS (ESI+, M+H): 481.2.

### Example 45

### (cis-1,3′)-N-Cyclopropyl-5,6-difluoro-N-[5-(2-methoxyethyl)-2,3-dimethylbenzyl]-3H-spiro[2-benzofuran-1,4′-piperidine]-3′-carboxamide

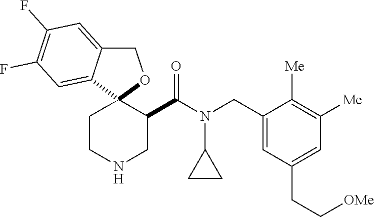

Prepared according to the procedure described in Example 15 but using Alkylation Reagent 10 (1.5 eq.) in place of benzyl bromide as the starting material in step 1. MS (ESI+, M+H): 485.4. ¹H NMR (500 MHz, CDCl₃): δ (ppm) 6.99 (dd, J=9.0, 7.0 Hz, 1H); 6.90 (s, 1H); 6.79 (t, J=9.0 Hz, 1H); 6.70 (s, 1H); 5.11 (d, J=12.0 Hz, 1H); 4.95 (d, J=12.0 Hz, 1H); 4.64 (d, J=15.0 Hz, 1H); 4.18 (d, J=14.5 Hz, 1H); 3.66 (dd, J=9.5, 4.0 Hz, 1H); 3.55 (t, J=7.0 Hz, 2H); 3.41 (dd, J=13.0, 9.5 Hz, 1H); 3.36 (s, 3H); 3.25-3.19 (m, 1H); 3.11 (dd, J=12.5, 3.5 Hz, 1H); 3.05-2.98 (m, 1H); 2.79-2.68 (m, 2H); 2.47-2.40 (m, 1H); 2.23 (s, 3H); 2.00 (s, 3H); 1.90-1.79 (m, 4H); 0.99-0.93 (m, 1H); 0.88-0.82 (m, 1H); 0.81-0.72 (m, 2H). Human Renin IC₅₀ (buffer): 0.1 nM. Human Renin IC₅₀ (plasma): 0.6 nM.

### Example 46

### (cis-1,3′)-N-Cyclopropyl-5,6-difluoro-N-[5-(3-methoxypropyl)-2,3-dimethylbenzyl]-3H-spiro[2-benzofuran-1,4′-piperidine]-3′-carboxamide

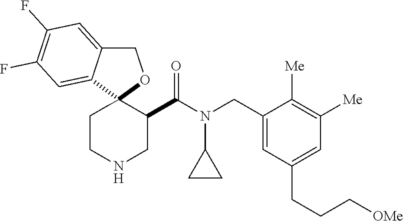

Prepared according to the procedure described in Example 15 but using Alkylation Reagent 11 (1.5 eq.) in place of benzyl bromide as the starting material in step 1. MS (ESI+, M+H): 499.4.

### Example 47

### (cis-1,3′)-N-{3-[2-(Acetylamino)ethyl]benzyl}-N-cyclopropyl-5,6-difluoro-3H-spiro[2-benzofuran-1,4′-piperidine]-3′-carboxamide

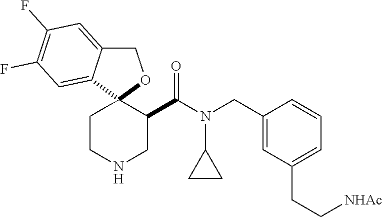

### Step 1

### tert-Butyl(cis-1,3′)-3′-{[[3-({[tert-butyl(dimethyl)silyl]oxy}methyl)benzyl](cyclopropyl)amino]carbonyl}-5,6-difluoro-1′H,3H-spiro[2-benzofuran-1,4′-piperidine]-1′-carboxylate

To a DMF solution (0.1 M) of Intermediate 1 (1 eq.) was added sodium hydride (60% (w/w) dispersion in oil, 1.5 eq.) and Alkylation Reagent 12 (1.5 eq.). The resulting suspension was stirred at RT for 6 h. The reaction mixture was then carefully quenched with sat. aq. NH₄Cl and then extracted with EtOAc. The combined organic extracts were dried over Na₂SO₄, filtered and the filtrate concentrated in vacuo. Purification of the crude product thus obtained by way of column chromatography (SiO₂, 9:1 (v/v) Hex:EtOAc→3:7 (v/v) Hex:EtOAc) afforded the title compound.

### Step 2

### tert-Butyl(cis-1,3′)-3′-({cyclopropyl[3-(hydroxymethyl)benzyl]amino}carbonyl)-5,6-difluoro-1′H,3H-spiro[2-benzofuran-1,4′-piperidine]-1′-carboxylate

To a THF solution (0.1 M) of tert-Butyl(cis-1,3′)-3′-{[[3-({[tert-butyl(dimethyl)silyl]oxy}methyl)benzyl] (cyclopropyl)amino]carbonyl}-5,6-difluoro-1′H,3H-spiro[2-benzofuran-1,4′-piperidine]-1′-carboxylate (1 eq.) from the previous step was added TBAF (1 M THF solution, 1.5 eq.). The resulting solution was stirred at RT for 1 h. The reaction mixture was then carefully quenched with sat. aq. NH₄Cl and then extracted with EtOAc. The combined organic extracts were washed further with brine, dried over Na₂SO₄, filtered and the filtrate concentrated in vacuo. Purification of the crude product thus obtained by way of column chromatography (SiO₂, 85:15 (v/v) Hex:EtOAc→1:9 (v/v) Hex:EtOAc) afforded the title compound.

### Step 3

### tert-Butyl(cis-1,3′)-3′-{[cyclopropyl(3-{[(methylsulfonyl)oxy]methyl}benzyl)amino]carbonyl)-5,6-difluoro-1′H,3H-spiro[2-benzofuran-1,4′-piperidine]-1′-carboxylate

To a dichloromethane solution (0.1 M) of tert-butyl(cis-1,3′)-3′-({cyclopropyl[3-(hydroxymethyl)benzyl]amino}carbonyl)-5,6-difluoro-1′H,3H-spiro[2-benzofuran-1,4′-piperidine]-1′-carboxylate (1 eq.) from the previous step was added triethylamine (1.2 eq.) and then methanesulfonyl chloride (1.1 eq.) at 0° C. The reaction mixture was stirred at 0° C. for 30 min before it was quenched with sat. aq. NaHCO₃. The aqueous layer was separated and back-extracted with dichloromethane. The combined organic extracts were then dried over Na₂SO₄, filtered and the filtrate concentrated in vacuo to furnish the title compound.

### Step 4

### tert-Butyl(cis-1,3′)-3′-{[[3-(cyanomethyl)benzyl](cyclopropyl)amino]carbonyl}-5,6-difluoro-1′H,3H-spiro[2-benzofuran-1,4′-piperidine]-1′-carboxylate

To a DMF solution (0.25 M) of tert-butyl(cis-1,3′)-3′-{[cyclopropyl(3-{[(methylsulfonyl)oxy]methyl}benzyl)amino]carbonyl)-5,6-difluoro-1′H,3H-spiro[2-benzofuran-1,4′-piperidine]-1′-carboxylate (1 eq.) from the previous step was added sodium cyanide (1.5 eq.). The reaction mixture was stirred at RT for 16 h before it was quenched with water and then extracted with EtOAc. The combined organic extracts were then dried over Na₂SO₄, filtered and the filtrate concentrated in vacuo. Purification of the crude product thus obtained by way of column chromatography (SiO₂, 4:1 (v/v) Hex:EtOAc→3:7 (v/v) Hex:EtOAc) afforded the title compound as a white foam.

### Step 5

### tert-Butyl(cis-1,3′)-3′-{[{3-[2-(acetylamino)ethyl]benzyl}(cyclopropyl)amino]carbonyl}-5,6-difluoro-1′H,3H-spiro[2-benzofuran-1,4′-piperidine]-1′-carboxylate

To a methanol solution (0.1 M) of tert-butyl(cis-1,3′)-3′-{[[3-(cyanomethyl)benzyl](cyclopropyl)amino]carbonyl}-5,6-difluoro-1′H,3H-spiro[2-benzofuran-1,4′-piperidine]-1′-carboxylate (1 eq.) from the previous step and cobalt(II) chloride hexahydrate (2 eq.) was added portionwise sodium borohydride (10 eq.). The reaction mixture was stirred at RT for 2.5 h before it was quenched with 1 N aq. NaOH and then diluted with dichloromethane. The insolubles were subsequently removed via filtration through a pad of celite and the filtrate was transferred to a separatory funnel. The aqueous layer was separated and back-extracted with dichloromethane. The combined organic extracts were dried over Na₂SO₄, filtered and the filtrate concentrated in vacuo. The crude amine thus obtained was then taken up in dichloromethane (0.1 M) and added Hünig's base (1.5 eq.) and acetyl chloride (1.2 eq.). The resulting solution was allowed to stir at RT for another 16 h. The reaction mixture was diluted with dichloromethane and washed sequentially with 1 N aq. NaOH and brine. The organic extract was then dried over Na₂SO₄, filtered and the filtrate concentrated in vacuo. Further purification by way of column chromatography (SiO₂, 4:1 (v/v) Hex:EtOAc→EtOAc) afforded the title compound.

### Step 6

### (cis-1,3′)-N-{3-[2-(Acetylamino)ethyl]benzyl}-N-cyclopropyl-5,6-difluoro-3H-spiro[2-benzofuran-1,4′-piperidine]-3′-carboxamide

To a CH₂Cl₂ solution (0.1 M) of tert-butyl(cis-1,3′)-3′-{[{3-[2-(acetylamino)ethyl]benzyl}(cyclopropyl)amino]carbonyl}-5,6-difluoro-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was then diluted with methanol and 10 N aq. NaOH. After 15 min, methanol was removed in vacuo and the aqueous layer was back-extracted with dichloromethane. The combined organic extracts were then dried over Na₂SO₄, filtered and the filtrate concentrated in vacuo. Purification of the crude product thus obtained by way of column chromatography (SiO₂, 95:5 (v/v) CH₂Cl₂: 2.0 M NH₃ in MeOH→4:1 (v/v) CH₂Cl₂: 2.0 M NH₃ in MeOH) afforded the title compound as a viscous gum. MS (ESI+, M+H): 484.2.

### Example 48

### (cis-1,3′)-N-Cyclopropyl-5,6-difluoro-N-{[2-(3-methoxypropyl)-4-quinolinyl]methyl}-3H-spiro[2-benzofuran-1,4′-piperidine]-3′-carboxamide

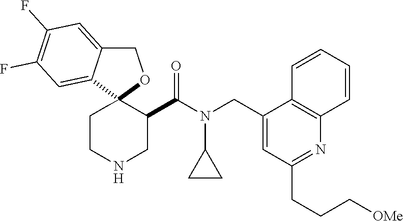

Prepared according to the procedure described in Example 15 but using Alkylation Reagent 13 (1.5 eq.) in place of benzyl bromide as the starting material in step 1. MS (ESI+, M+H): 522.4. ¹H NMR (500 MHz, CDCl₃): δ (ppm) 8.07 (d, J=8.5 Hz, 1H); 7.99 (d, J=8.4 Hz, 1H); 7.70 (t, J=7.7 Hz, 1H); 7.49 (t, J=7.6 Hz, 1H); 7.01 (s, 1H); 6.92 (t, J=8.0 Hz, 1H); 6.44 (t, J=8.0 Hz, 1H); 5.18 (d, J=15.2 Hz, 1H); 4.95 (d, J=12.2 Hz, 1H); 4.86 (d, J=12.2 Hz, 1H); 4.49 (d, J=15.2 Hz, 1H); 3.69-3.64 (m, 1H); 3.50 (t, J=6.7 Hz, 2H); 3.43 (dd, J=12.6, 9.5 Hz, 1H); 3.39 (s, 3H); 3.25-3.18 (m, 1H); 3.12 (dd, J=12.6, 3.7 Hz, 1H); 3.04-2.91 (m, 3H); 2.50-2.45 (m, 1H); 2.15-2.03 (m, 2H); 1.92-1.76 (m, 2H); 1.73 (br, 2H); 1.11-1.04 (m, 1H); 1.02-0.94 (m, 1H); 0.90-0.79 (m, 2H). Human Renin IC₅₀ (buffer): 0.7 nM. Human Renin IC₅₀ (plasma): 2.3 nM.

### Example 49

### (cis-1,3′)-N-{3-[(Acetylamino)methyl]benzyl}-N-cyclopropyl-5,6-difluoro-3H-spiro[2-benzofuran-1,4′-piperidine]-3′-carboxamide

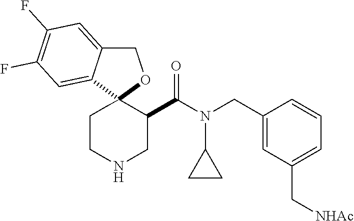

### Step 1

### tert-Butyl(cis-1,3′)-3′-{[(3-cyanobenzyl)(cyclopropyl)amino]carbonyl}-5,6-difluoro-1′H,3H-spiro[2-benzofuran-1,4′-piperidine]-1′-carboxylate

To a DMF solution (0.1 M) of Intermediate 1 (1 eq.) was added sodium hydride (60% (w/w) dispersion in oil, 1.5 eq.) and Alkylation Reagent 14 (1.5 eq.). The resulting suspension was stirred at RT for 6 h. The reaction mixture was then carefully quenched with sat. aq. NH₄Cl and then extracted with EtOAc. The combined organic extracts were dried over Na₂SO₄, filtered and the filtrate concentrated in vacuo. Purification of the crude product thus obtained by way of column chromatography (SiO₂, 4:1 (v/v) Hex:EtOAc→3:7 (v/v) Hex:EtOAc) afforded the title compound as a white foam.

### Step 2

### tert-Butyl(cis-1,3′)-3′-{[{3-[(acetylamino)methyl]benzyl}(cyclopropyl)amino]carbonyl}-5,6-difluoro-1′H,3H-spiro[2-benzofuran-1,4′-piperidine]-1′-carboxylate

To a methanol solution (0.05 M) of tert-butyl(cis-1,3′)-3′-{[(3-cyanobenzyl)(cyclopropyl)amino]carbonyl}-5,6-difluoro-1′H,3H-spiro[2-benzofuran-1,4′-piperidine]-1′-carboxylate (1 eq.) from the previous step and cobalt(II) chloride hexahydrate (2 eq.) was added portionwise sodium borohydride (10 eq.). The reaction mixture was stirred at RT for 3 h before it was quenched with 1 N aq. NaOH and then diluted with dichloromethane. The insolubles were subsequently removed via filtration through a pad of celite and the filtrate was transferred to a separatory funnel. The aqueous layer was separated and back-extracted with dichloromethane. The combined organic extracts were dried over Na₂SO₄, filtered and the filtrate concentrated in vacuo. The crude amine thus obtained was then taken up in dichloromethane (0.1 M) and added Hünig's base (1.5 eq.) and acetyl chloride (1.2 eq.). The resulting solution was allowed to stir at RT for another 16 h. The reaction mixture was diluted with dichloromethane and washed sequentially with sat. aq. NaHCO₃, sat. aq. NH₄Cl and brine. The organic extract was then dried over Na₂SO₄, filtered and the filtrate concentrated in vacuo to furnish the title compound as a pale green glass.

### Step 3

### (cis-1,3′)-N-{3-[(Acetylamino)methyl]benzyl}-N-cyclopropyl-5,6-difluoro-3H-spiro[2-benzofuran-1,4′-piperidine]-3′-carboxamide

To a CH₂Cl₂ solution (0.1 M) of tert-Butyl(cis-1,3′)-3′-{[{3-[(acetylamino)methyl]benzyl}(cyclopropyl)amino]carbonyl}-5,6-difluoro-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 2 h. Following the removal of the volatiles in vacuo, the resulting residue was then diluted with dichloromethane and 1 N aq. NaOH. The organic extract was then separated, dried over Na₂SO₄, filtered and the filtrate concentrated in vacuo. Purification of the crude product thus obtained by way of column chromatography (SiO₂, CH₂Cl₂→5:1 (v/v) CH₂Cl₂: 2.0 M NH₃ in MeOH) afforded the title compound as a white foam. MS (ESI+, M+H): 470.2.

### Example 50

### (cis-1,3′)-N-[(6-Bromo-8-quinolinyl)methyl]-N-cyclopropyl-5,6-difluoro-3H-spiro[2-benzofuran-1,4′-piperidine]-3′-carboxamide

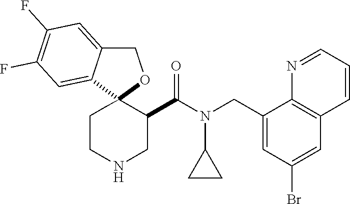

Prepared according to the procedure described in Example 15 but using Alkylation Reagent 15 (1.5 eq.) in place of benzyl bromide as the starting material in step 1. MS (ESI+, M+H): 528.1.

### Example 51

### (cis-1,3′)-N-[(2-Bromo-3-pyridinyl)methyl]-N-cyclopropyl-5,6-difluoro-3H-spiro[2-benzofuran-1,4′-piperidine]-3′-carboxamide

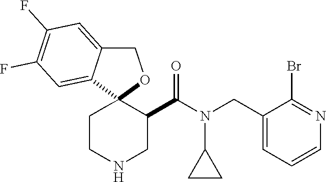

Prepared according to the procedure described in Example 15 but using 2-bromo-3-(bromomethyl)pyridine (1.5 eq.) in place of benzyl bromide as the starting material in step 1. MS (ESI+, M+H): 478.1.

### Example 52

### (cis-1,3′)-N-[(6-Chloro-3-pyridinyl)methyl]-N-cyclopropyl-5,6-difluoro-3H-spiro[2-benzofuran-1,4′-piperidine]-3′-carboxamide

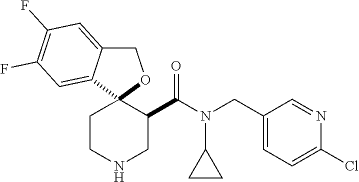

Prepared according to the procedure described in Example 15 but using 2-chloro-5-(chloromethyl)pyridine (1.5 eq.) in place of benzyl bromide as the starting material in step 1. MS (ESI+, M+H): 434.2.

### Example 53

### (cis-1,3′)-N-[(2-Chloro-3,5-bis(3-methoxypropyl)benzyl]-N-cyclopropyl-5,6-difluoro-3H-spiro[2-benzofuran-1,4′-piperidine]-3′-carboxamide

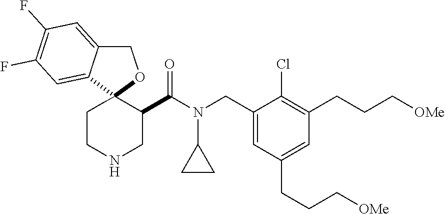

Prepared according to the procedure described in Example 15 but using Alkylation Reagent 16 (1.5 eq.) in place of benzyl bromide as the starting material in step 1. MS (ESI+, M+H): 577.3. ¹H NMR (500 MHz, CDCl₃): δ (ppm) 7.10-6.95 (m, 1H); 6.94 (s, 1 H); 6.80 (t, J=8.1 Hz, 1H); 6.66 (d, J=8.7 Hz, 1H); 5.09 (d, J=12.1 Hz, 1H); 4.95 (d, J=12.3 Hz, 1H); 4.68 (d, J=15.5 Hz, 1H); 4.31 (d, J=15.4 Hz, 1H); 3.68 (dd, J=9.3, 3.7 Hz, 1H); 3.45-3.33 (m, 11H); 3.26-3.19 (m, 1H); 3.13 (dd, J=12.7, 3.9 Hz, 1H); 3.08-2.98 (m, 1 H); 2.83-2.68 (m, 2H); 2.61-2.48 (m, 3H); 1.96-1.76 (m, 6H); 1.73 (br, 2H); 1.00-0.90 (m, 1H); 0.91-0.80 (m, 1H); 0.82-0.68 (m, 2H). Human Renin IC₅₀ (buffer): 0.1 nM. Human Renin IC₅₀ (plasma): 1.3 nM

### Example 54

### (cis-1,3′)-N-Cyclopropyl-5,6-difluoro-N-{[6-(3-methoxypropyl)-8-quinolinyl]methyl}-3H-spiro[2-benzofuran-1,4′-piperidine]-3′-carboxamide

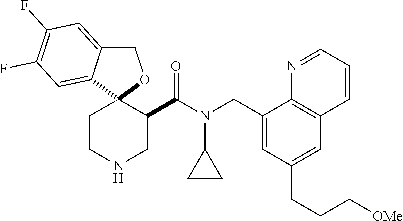

Prepared according to the procedure described in Example 15 but using Alkylation Reagent 17 (1.5 eq.) in place of benzyl bromide as the starting material in step 1. MS (ESI+, M+H): 522.4. NMR (500 MHz, CDCl₃): δ (ppm) 8.76 (dd, J=4.2, 1.7 Hz, 1H); 8.05 (dd, J=8.2, 1.7 Hz, 1H); 7.47 (s, 1H); 7.36 (dd, J=8.2, 4.2 Hz, 1H); 7.24 (s, 1H); 6.91 (dd, J=9.5, 6.8 Hz, 1H); 6.49 (t, J=8.1 Hz, 1H); 5.04 (t, J=14.9 Hz, 1H); 4.94 (t, J=13.3 Hz, 2H); 4.83 (d, J=12.1 Hz, 1H); 3.74-3.62 (m, 1H); 3.46-3.39 (m, 3H); 3.38 (s, 3H); 3.25-3.15 (m, 1H); 3.16-3.09 (m, 1H); 3.04-2.89 (m, 1H); 2.87-2.70 (m, 2H); 2.69-2.63 (m, 1 H); 1.99-1.88 (m, 2H); 1.81 (br, 2H); 1.01-0.94 (m, 1H); 0.91-0.85 (m, 1H); 0.83-0.71 (m, 2H). Human Renin IC₅₀ (buffer): 0.1 nM. Human Renin IC₅₀ (plasma): 3.5 nM

### Example 55

### (cis-1,3′)-N-[5-Bromo-2-methyl-3-(trifluoromethyl)benzyl]-N-cyclopropyl-5,6-difluoro-3H-spiro[2-benzofuran-1,4′-piperidine]-3′-carboxamide

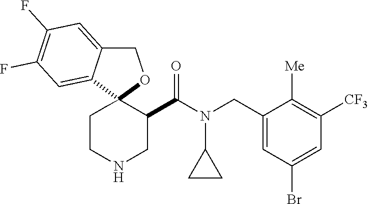

Prepared according to the procedure described in Example 15 but using Alkylation Reagent 18 (1.5 eq.) in place of benzyl bromide as the starting material in step 1. MS (ESI+, M+H): 559.2.

### Example 56

### (cis-1,3′)-N-cyclopropyl-5,6-difluoro-N-[(4-fluoro-1H-indol-3-yl)methyl]-3H-spiro[2-benzofuran-1,4′-piperidine]-3′-carboxamide

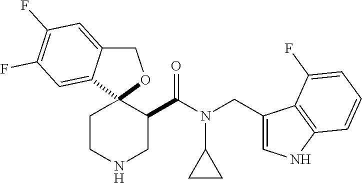

### Step 1

### tert-Butyl(cis-1,3′)-3′-{[{[1-(tert-butoxycarbonyl)-4-fluoro-1H-indol-3-yl]methyl}(cyclopropyl)amino]carbonyl}-5,6-difluoro-1′H,3H-spiro[2-benzofuran-1,4′-piperidine]-1′-carboxylate

To a DMF solution (0.1 M) of Intermediate 1 (1 eq.) was added sodium hydride

(60% (w/w) dispersion in oil, 1.5 eq.) and Alkylation Reagent 19 (0.1 M THF solution, 1.5 eq.). The resulting suspension was stirred at RT for 16 h. The reaction mixture was then carefully quenched with sat. aq. NH₄Cl and then extracted with EtOAc. The combined organic extracts were washed with brine, dried over Na₂SO₄, filtered and the filtrate concentrated in vacuo. Purification of the crude product thus obtained by way of column chromatography (SiO₂, 95:5 (v/v) Hex:EtOAc→EtOAc) afforded the title compound.

### Step 2

### (cis-1,3′)-N-Cyclopropyl-5,6-difluoro-N-[(4-fluoro-1H-indol-3-yl)methyl]-3H-spiro[2-benzofuran-1,4′-piperidine]-3′-carboxamide

To a dichloromethane (0.2 M) solution of tert-butyl(cis-1,3′)-3′-{[{[1-(tert-butoxycarbonyl)-4-fluoro-1H-indol-3-yl]methyl}(cyclopropyl)amino]carbonyl}-5,6-difluoro-1′H,3H-spiro[2-benzofuran-1,4′-piperidine]-1′-carboxylate (1 eq.) from the previous step was added zinc chloride (1 M ether solution, 3 eq.). The reaction mixture was allowed to stir at RT for 3 h after which 1 N aq. NaOH was added. The aqueous layer was separated and back-extracted with CH₂Cl₂. The combined organic extracts were washed further with brine, dried over Na₂SO₄, filtered and the filtrate concentrated in vacuo. The residue thus obtained was then taken up in 1:1 (v/v) THF:MeOH (0.14 M). To this was added lithium hydroxide (2 M aq. solution, 2 eq.) and the resulting mixture was heated at 85° C. for 10 min. After the reaction mixture was cooled to RT, it was diluted with water and then extracted with EtOAc. The combined organic extracts were washed further with brine, dried over Na₂SO₄, filtered and the filtrate concentrated in vacuo. Purification of the crude product thus obtained by way of column chromatography (SiO₂, 95:5 (v/v) CH₂Cl₂: 2.0 M NH₃ in MeOH) afforded the title compound as a white solid. MS (ESI+, M+H): 456.2. ¹H NMR (500 MHz, CDCl₃): δ (ppm) 8.88 (s, 1H), 7.19 (d, J=8.1 Hz, 1H), 7.09 (td, J=8.0, 4.9 Hz, 1H), 6.99 (s, 1H), 6.91 (t, J=8.0 Hz, 1H), 6.73 (dd, J=11.3, 7.8 Hz, 1H), 6.19 (t, J=8.0 Hz, 1H), 4.95 (d, J=14.5 Hz, 1H), 4.71 (d, J=12.0 Hz, 1H), 4.64 (d, J=12.0 Hz, 1H), 4.29 (d, J=14.5 Hz, 1H), 3.63 (dd, J=10.7, 3.8 Hz, 1H), 3.44 (t, J=11.7 Hz, 1H), 3.26-3.15 (m, 1H), 3.07-3.02 (m, 2H), 2.39 (s, 1H), 1.88-1.74 (m, 2H), 1.14-1.05 (m, 1H), 0.96-0.89 (m, 1H), 0.84-0.73 (m, 2H). Human Renin IC₅₀ (buffer): 1.2 nM. Human Renin IC₅₀ (plasma): 4.5 nM.

### Example 57

### (cis-1,3′)-N-Cyclopropyl-5,6-difluoro-N-(1H-pyrrolo[2,3-b]pyridine-3-ylmethyl)-3H-spiro[2-benzofuran-1,4′-piperidine]-3′-carboxamide

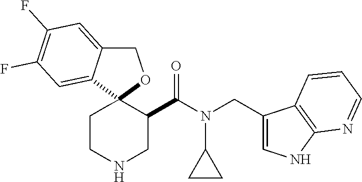

### Step 1

### tert-Butyl(cis-1,3′)-3′-{[{[1-(tert-butoxycarbonyl)-1H-pyrrolo[2,3-b]pyridin-3-yl]methyl}(cyclopropyl)amino]carbonyl}-5,6-difluoro-1′H,3H-spiro[2-benzofuran-1,4′-piperidine]-1′-carboxylate

To a DMF solution (0.1 M) of Intermediate 1 (1 eq.) was added sodium hydride (60% (w/w) dispersion in oil, 1.5 eq.) and Alkylation Reagent 20 (1.5 eq.). The resulting suspension was stirred at RT for 14 h. The reaction mixture was then carefully quenched with sat. aq. NH₄Cl and then extracted with EtOAc. The combined organic extracts were washed with brine, dried over Na₂SO₄, filtered and the filtrate concentrated in vacuo. Purification of the crude product thus obtained by way of column chromatography (SiO₂, 95:5 (v/v) Hex:EtOAc→EtOAc) afforded the title compound.

### Step 2

### (cis-1,3′)-N-Cyclopropyl-5,6-difluoro-N-(1H-pyrrolo[2,3-b]pyridine-3-ylmethyl)-3H-spiro[2-benzofuran-1,4′-piperidine]-3′-carboxamide

To a dichloromethane (0.2 M) solution of tert-butyl(cis-1,3′)-3′-{[{[1-(tert-butoxycarbonyl)-1H-pyrrolo[2,3-b]pyridin-3-yl]methyl}(cyclopropyl)amino]carbonyl}-5,6-difluoro-1′H,3H-spiro[2-benzofuran-1,4′-piperidine]-1′-carboxylate (1 eq.) from the previous step was added zinc chloride (1 M ether solution, 3 eq.). The reaction mixture was allowed to stir at RT for 15 h after which 1 N aq. NaOH was added. The aqueous layer was separated and back-extracted with CH₂Cl₂. The combined organic extracts were washed further with brine, dried over Na₂SO₄, filtered and the filtrate concentrated in vacuo. Purification of the crude product thus obtained by way of column chromatography (SiO₂, CH₂Cl₂→5:1 (v/v) CH₂Cl₂: 2.0 M NH₃ in MeOH) afforded the title compound. MS (ESI+, M+H): 439.2.

### Example 58

### (cis-1,3′)-N-Cyclopropyl-5,6-difluoro-N-[(4-fluoro-1-naphthyl)methyl]-3H-spiro[2-benzofuran-1,4′-piperidine]-3′-carboxamide

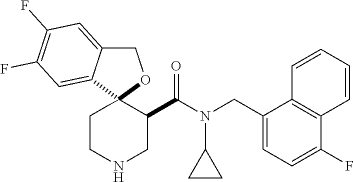

### Step 1

### 4-Fluoro-1-naphthaldehyde

To a dichloromethane solution (0.2 M) of (4-fluoro-1-naphthyl)methanol (1 eq.) was added DMP (1.2 eq.) at 0° C. The resulting mixture was allowed to stir at 0° C. for 1 h after which sat. aq. NaHCO₃ and 1 M aq. NaHSO₃ were added. The aqueous layer was separated and back-extracted with EtOAc. The combined organic extracts were washed further with brine, dried over Na₂SO₄, filtered and the filtrate concentrated in vacuo to furnish the title compound as a white solid.

### Step 2

### N-[(4-Fluoro-1-naphthyl)methyl]cyclopropanamine

To a dichloromethane solution (0.2 M) of 4-fluoro-1-naphthaldehyde (1 eq.) from the previous step was added magnesium sulfate (1.5 eq.) and cyclopropylamine (2 eq.). The resulting suspension was stirred at RT for 18 h after which sodium borohydride (2 eq.) and methanol were added. After 1 h of stirring at RT, the reaction mixture was quenched with sat. aq. NaHCO₃. The aqueous layer was separated and back-extracted with EtOAc. The combined organic extracts were washed further with brine, dried over Na₂SO₄, filtered and the filtrate concentrated in vacuo. Purification of the crude product thus obtained by way of column chromatography (SiO₂, 95:5 (v/v) Hex:EtOAc→EtOAc) afforded the title compound as a yellow oil.

### Step 3

### tert-Butyl(cis-1,3′)-3′-({cyclopropyl[(4-fluoro-1-naphthyl)methyl]amino}carbonyl)-5,6-difluoro-1′H,3H-spiro[2-benzofuran-1,4′-piperidine]-1;-carboxylate

To a THF solution (0.15 M) of 1′-tert-butyl 3′-ethyl (1R,3′S)-5,6-difluoro-1′H,3H-spiro[2-benzofuran-1,4′-piperidine]-1′,3′-dicarboxylate (1 eq., Intermediate 1, Step 4) and N-[(4-fluoro-1-naphthyl)methyl]cyclopropanamine (2.7 eq.) from the previous step was added isopropyl magnesium chloride (2 M THF solution, 3.2 eq.) at −40° C. dropwise. Following the completion of addition, the reaction mixture was allowed to warm to RT over 2 h after which it was quenched with sat. aq. NH₄Cl. The aqueous layer was separated and back-extracted with EtOAc. The combined organic extracts were washed further with water and brine, dried over Na₂SO₄, filtered and the filtrate concentrated in vacuo. Purification of the crude product thus obtained by way of column chromatography (SiO₂, 95:5 (v/v) Hex:EtOAc→EtOAc) afforded the title compound.

### Step 4

### (cis-1,3′)-N-Cyclopropyl-5,6-difluoro-N-[(4-fluoro-1-naphthyl)methyl]-3H-spiro[2-benzofuran-1,4′-piperidine]-3′-carboxamide

To a CH₂Cl₂ solution (0.1 M) of tert-butyl(cis-1,3′)-3′-({cyclopropyl[(4-fluoro-1-naphthyl)methyl]amino}carbonyl)-5,6-difluoro-1′H,3H-spiro[2-benzofuran-1,4′-piperidine]-1;-carboxylate (1 eq.) from the previous step was added HCl (4.0 M dioxane solution, 30 eq.). The resulting solution was stirred at RT for 2 h. Following the removal of the volatiles in vacuo, the resulting residue was then diluted with dichloromethane and 1 N aq. NaOH. The organic extract was then separated, dried over Na₂SO₄, filtered and the filtrate concentrated in vacuo. Purification of the crude product thus obtained by way of column chromatography (SiO₂, CH₂Cl₂→4:1 (v/v) CH₂Cl₂: 2.0 M NH₃ in MeOH) afforded the title compound. MS (ESI+, M+H): 467.6.

### Example 59

### (cis-1,3′)-N-[2-Chloro-3-(trifluoromethyl)benzyl]-N-cyclopropyl-5,6-difluoro-3-oxo-3H-spiro[2-benzofuran-1,4′-piperidine]-3′-carboxamide

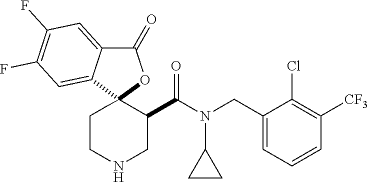

### Step 1

### tert-Butyl(cis-1,3′)-3′-{[[2-chloro-3-(trifluoromethyl)benzyl](cyclopropyl)amino]carbonyl}-5,6-difluoro-3-oxo-1′H,3H-spiro[2-benzofuran-1,4′-piperidine]-1′-carboxylate

To a DMF solution (0.1 M) of Intermediate 2 (1 eq.) was added sodium hydride (60% (w/w) dispersion in oil, 1.2 eq.) and 2-chloro-3-(trifluoromethyl)benzyl bromide (1.2 eq.). The resulting suspension was stirred at 45° C. for 12 h. After cooling to RT, the reaction mixture was diluted with ether and washed sequentially with 10% aq. HCl, water and brine. The organic extract was then dried over Na₂SO₄, filtered and the filtrate concentrated in vacuo. Purification of the crude product thus obtained by way of column chromatography (SiO₂, 9:1 (v/v) Hex:EtOAc→3:7 (v/v) Hex:EtOAc) afforded the title compound as a white solid.

### Step 2

### (cis-1,3′)-N-[2-Chloro-3-(trifluoromethyl)benzyl]-N-cyclopropyl-5,6-difluoro-3-oxo-3H-spiro[2-benzofuran-1,4′-piperidine]-3′-carboxamide

To a CH₂Cl₂ solution (0.06 M) of tert-butyl(cis-1,3′)-3′-{[[2-chloro-3-(trifluoromethyl)benzyl](cyclopropyl)amino]carbonyl}-5,6-difluoro-3-oxo-1′H,3H-spiro[2-benzofuran-1,4′-piperidine]-F-carboxylate (1 eq.) from the previous step was added HCl (4.0 M dioxane solution, 30 eq.). The resulting solution was stirred at RT for 3 h. Following the removal of the volatiles in vacuo, the resulting residue was directly loaded onto a SiO₂ column packed with 95:5 (v/v) CH₂Cl₂: 2.0 M NH₃ in MeOH. Elution with the same solvent system furnished the title compound as a white solid. MS (ESI+, M+H): 515.1.

### Example 60

### (cis-1,3′)-N-Cyclopropyl-5,6-difluoro-N-[2-methyl-3-(trifluoromethyl)benzyl]-3-oxo-3H-spiro[2-benzofuran-1,4′-piperidine]-3′-carboxamide

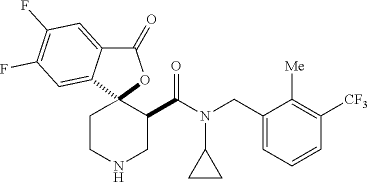

Prepared according to the procedure described in Example 59 but using 2-methyl-3-(trifluoromethyl)benzyl bromide (1.2 eq.) in place of 2-chloro-3-(trifluoromethyl)benzyl bromide as the starting material in step 1. MS (ESI+, M+H): 495.2.

### Example 61

### (cis-1,3′)-N-Cyclopropyl-5,6-difluoro-3-oxo-N-[2-(trifluoromethyl)benzyl]-3H-spiro[2-benzofuran-1,4′-piperidine]-3′-carboxamide

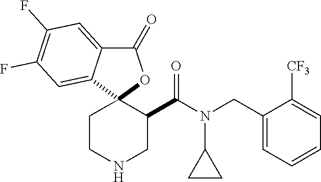

Prepared according to the procedure described in Example 59 but using 2-(trifluoromethyl)benzyl bromide (1.2 eq.) in place of 2-chloro-3-(trifluoromethyl)benzyl bromide as the starting material in step 1. MS (ESI+, M+H): 481.3.

### Example 62

### Assays Demonstrating Biological Activity

### Inhibition of Human Recombinant Renin

Human recombinant renin (Proteos) in 50 mM MOPS pH 7.4, 100 mM NaCl, 0.002% Tween 20 at a final concentration of 100 μM is incubated with inhibitors from a 50 fold concentrated DMSO solution and 6 μM of an internally-quench fluorescent peptide: DNP-Lys-His-Pro-Phe-His-Leu-Val-Ile-His-D,L-Amp (SEQ ID NO: 1); Paschalidou K. et al., Biochem J., 2004, 382, 1031). The reactions take place in a Costar 384 well black plate (#3573) at 37° C. for 3 hours. Fluorescence is measured at times 0 and 3 hours with a SpectraMax Gemini EM reader set at an excitation wavelength of 328 nm and at an emission wavelength of 388 nm. Background fluorescence at t=0 is subtracted from the measurement at t=3 hours. Inhibitory activity of the compounds is expressed as IC₅₀.

### Inhibition of Renin in Human Plasma

Human EDTA-collected plasma is rapidly thawed in warm water and centrifuged at 2900 g for 15 minutes at 4° C. The supernatant is collected and recombinant renin (Proteos) is added at a final concentration of 1 nM. The plasma is transferred to a Costar black 384 well plate (#3573). Renin inhibitors are added from a 17.5 fold concentrated DMSO solution and pre-incubated at 37° C. for 10 minutes. The internally-quench fluorescent peptide QXL520™-Lys-His-Pro-Phe-His-Leu-Val-Ile-His-Lys (5-FAM) (Anaspec) is diluted in 3M Tris pH 7.2, 200 mM EDTA and added to the plasma. The final concentrations are: 6 μM substrate, 342 mM Tris, 23 mM EDTA. The plate is incubated at 37° C. for 1 hour. The plate is read in a SpectraMax Gemini EM reader set at an excitation wavelength of 490 nm and an emission wavelength of 520 nM at times 0 and 1 hour. Background fluorescence at t=0 is subtracted from the measurement at t=1 hour. Inhibitory activity of the compounds is expressed as IC₅₀.

### In Vivo Animal Model

Female double transgenic rats were purchased from RCC Ltd, Füllingsdorf, Switzerland. All animals were maintained under identical conditions and had free access to normal pelleted rat chow and water. Rats were initially treated with enalapril (1 mg/kg/day) during 2 months. After approximately two weeks following cessation of enalapril treatment the double transgenic rats become hypertensive and reach mean arterial blood pressures in the range of 160-170 mmHg.

Transmitter implantation—The rats were anaesthetized with a mixture of 90 mg/kg ketamine-HCl (Ketavet, Parke-Davis, Berlin FRG) and 10 mg/kg xylazine (Rompun, Bayer, Leverkusen, FRG) i.p. The pressure transmitter was implanted under aseptic conditions into the peritoneal cavity with the sensing catheter placed in the descending aorta below the renal arteries pointing upstream. The transmitter was sutured to the abdominal musculature and the skin closed.

Telemetry-System—Telemetry units were obtained from Data Sciences (St. Paul, Minn.). The implanted sensor consisted of a fluid-filled catheter (0.7 mm diameter, 8 cm long; model TA11PA-C40) connected to a highly stable low-conductance strain-gauge pressure transducer, which measured the absolute arterial pressure relative to a vacuum, and a radio-frequency transmitter. The tip of the catheter was filled with a viscous gel that prevents blood reflux and was coated with an antithrombogenic film to inhibit thrombus formation. The implants (length=2.5 cm, diameter=1.2 cm) weighted 9 g and have a typical battery life of 6 months. A receiver platform (RPC-1, Data Sciences) connected the radio signal to digitized input that was sent to a dedicated personal computer (Compaq, deskpro). Arterial pressures were calibrated by using an input from an ambient-pressure reference (APR-1, Data Sciences). Systolic, mean and diastolic blood pressure was expressed in millimeter of mercury (mmHg).

Hemodynamic measurements—Double transgenic rats with implanted pressure transmitters were dosed by oral gavage with vehicle or 10 mg/kg of the test substance (n=6 per group) and the mean arterial blood pressure was continuously monitored. The effect of the test substance is expressed as maximal decrease of mean arterial pressure (MAP) in the treated group versus the control group.

### RESULTS

Compounds in accordance herewith were active, exhibiting an IC50<1 μM in both renin buffer and plasma assays. Data for some representative compounds can be found in Examples 17, 18, 21, 26, 32, 38, 45, 48, 53, 54, and 56 above.

1-8. (canceled)

9. A compound of structural formula I: 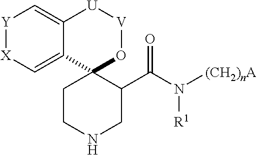
and pharmaceutically acceptable salts thereof, wherein:
n, for each instance in which it occurs, is independently 0, 1, or 2;
R¹ is hydrogen, C₁₋₆-alkyl or C₃₋₆-cycloalkyl, wherein said C₁₋₆-alkyl or C₃₋₆-cycloalkyl group is unsubstituted or independently substituted with 1-3 halogens;
A is (i) a five- or six-membered saturated or unsaturated heterocyclic or carbocyclic monocyclic ring or (ii) a five- or six-membered saturated or unsaturated heterocyclic or carbocyclic ring which is fused to another five- or six-membered saturated or unsaturated heterocyclic or carbocyclic ring,
wherein the heterocyclic rings of (i) or (ii) contain from 1-3 heteroatoms which are independently N, O or S, wherein each N is optionally in the form of an oxide and each S is optionally in the form of an oxide selected from the group consisting of S(═O) and S(═O)₂,
wherein the heterocyclic or carbocyclic rings of (i) or (ii) are unsubstituted or substituted by 1-4 radicals independently selected:
(1) halogen,
(2) cyano,
(3) C₁₋₆ alkyl,
(4) Cl₁₋₆ alkanoyl,
(5) C₁₋₆ alkoxy,
(6) C₂₋₆ alkenyl,
(7) C₃₋₆ cycloalkyl,
(8) carboxy-C₁₋₆ alkyl, or
(9) carboxy-C₃₋₆ cycloalkyl,
wherein substituents (3)-(9) are unsubstituted or substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or W, wherein W is morpholine, oxomorpholine, pyrrolidine, succinimide, acylmorpholine, or thiomorpholine 1,1-dioxide;
R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group is unsubstituted or independently substituted with 1-3 halogens;
R³ is hydrogen, C₁₋₄ alkyl or C₃₋₆ cycloalkyl, wherein said C₁₋₄ alkyl or C₃₋₆ cycloalkyl group is unsubstituted or independently substituted with 1-3 halogens;
V is a bond or —(C═O)—, —CH(OH)—, —CH₂— or ═CH—;
U is a bond or —CH₂—, or for the case when V is ═CH—, U is —CH═;
X is ═CH—, ═CF—, ═C(OR³)—, or —C═O—; and
Y is ═CH—, ═CF—, ═N—, or for the case when X is —C═O—, Y is —N(R³)—.

10. The compound of claim 9 having formula Ia: 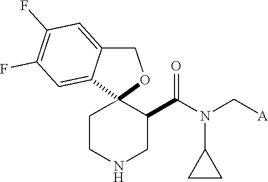
wherein A is (i) a five- or six-membered saturated or unsaturated heterocyclic or carbocyclic monocyclic ring or (ii) a five- or six-membered saturated or unsaturated heterocyclic or carbocyclic ring which is fused to another five- or six-membered saturated or unsaturated heterocyclic or carbocyclic ring,
wherein the heterocyclic rings of (i) or (ii) contain from 1-3 heteroatoms, independently selected from N, O and S, wherein each N is optionally in the form of an oxide and each S is optionally in the form of an oxide selected from the group consisting of S(═O) and S(═O)₂,
wherein the heterocyclic or carbocyclic rings of (i) or (ii) is unsubstituted or substituted by 1-4 radicals independently selected from:
(1) halogen,
(2) cyano,
(3) C₁₋₆ alkyl,
(4) C₁₋₆ alkanoyl,
(5) C₁₋₆ alkoxy,
(6) C₂₋₆ alkenyl,
(7) C₃₋₆ cycloalkyl,
(8) carboxy-C₁₋₆ alkyl, or
(9) carboxy-C₃₋₆ cycloalkyl,
wherein substituents (3)-(9) are unsubstituted or independently substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or W, wherein W is morpholine, oxomorpholine, pyrrolidine, succinimide, acylmorpholine, or thiomorpholine 1,1-dioxide;
R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group is unsubstituted or independently substituted with 1-3 halogens;
R³ is hydrogen, C₁₋₄ alkyl or C₃₋₆ cycloalkyl, wherein said C₁₋₄ alkyl or C₃₋₆ cycloalkyl group is unsubstituted or independently substituted with 1-3 halogens.

11. The compound of claim 9 wherein A is
(1) substituted or unsubstituted benzyl, or substituted or unsubstituted naphthyl; and
(2) substituted or unsubstituted quinolinyl, substituted or unsubstituted isoquinolinyl, substituted or unsubstituted pyridinyl, substituted or unsubstituted benzopyridinyl, or substituted or unsubstituted indolyl.

12. The compound of claim 9 wherein A is unsubstituted or substituted benzyl or benzoate, with one or more substituents selected from halo, C₁₋₆ alkyl, C₁₋₆haloalkyl, C₁₋₆ alkoxy, C₁₋₆haloalkoxy, or acetylamino.

13. A compound of claim 9, which is
(cis-1,3′)-N-Cyclopropyl-N-[3-(2-methoxyethoxy)-5-(3-methoxypropyl)benzyl]-3-oxo-3H-spiro[2-benzofuran-1,4′-piperidine]-3′-carboxamide;
(cis-1,3′)-N-Cyclopropyl-3-hydroxy-N-[3-(2-methoxyethoxy)-5-(3-methoxypropyl)benzyl]-3H-spiro[2-benzofuran-1,4′-piperidine]-3′-carboxamide;
(cis-1,3′)-N-Cyclopropyl-N-[3-(2-methoxyethoxy)-5-(3-methoxypropyl)benzyl]-3H-spiro[2-benzofuran-1,4′-piperidine]-3′-carboxamide;
(cis-1,3′)-N-[2-Chloro-5-(2-methoxyethyl)benzyl]-N-cyclopropyl-3-oxo-3H-spiro[2-benzofuran-1,4′-piperidine]-3′-carboxamide;
(cis-1,3′)-N-Cyclopropyl-N-[3-(2-methoxyethoxy)-5-(3-methoxypropyl)benzyl]-3,4-dihydrospiro[isochromene-1,4′-piperidine]-3′-carboxamide;
(cis-1,3′)-N-Cyclopropyl-5,6-difluoro-N-[3-(2-methoxyethoxy)-5-(3-methoxypropyl)benzyl]-3-oxo-3H-spiro[2-benzofuran-1,4′-piperidine]-3′-carboxamide;
(cis-1,3′)-N-[2-Chloro-5-(2-methoxyethyl)benzyl]-N-cyclopropyl-5,6-difluoro-3-oxo-3H-spiro[2-benzofuran-1,4′-piperidine]-3′-carboxamide;
(cis-1,3′)-N-Cyclopropyl-5,6-difluoro-N-[3-(2-methoxyethoxy)-5-(3-methoxypropyl)benzyl]-3H-spiro[2-benzofuran-1,4′-piperidine]-3′-carboxamide;
(cis-1,3′)-N-[2-Chloro-5-(2-methoxyethyl)benzyl]-N-cyclopropyl-5,6-difluoro-3H-spiro[2-benzofuran-1,4′-piperidine]-3′-carboxamide;
(cis-1,3′)-N-Cyclopropyl-N-[3-(2-methoxyethoxy)-5-(3-methoxypropyl)benzyl]spiro[isochromene-1,4′-piperidine]-3′-carboxamide;
(cis-1,3′)-N-[2-Chloro-5-(2-methoxyethyl)benzyl]-N-cyclopropylspiro[isochromene-1,4′-piperidine]-3′-carboxamide;
(cis-1,3′)-N-Cyclopropyl-N-[3-(2-methoxyethoxy)-5-(3-methoxypropyl)benzyl]-3-oxo-3,4-dihydrospiro[isochromene-1,4′-piperidine]-3′-carboxamide;
(cis-1,3′)-N-[2-Chloro-5-(2-methoxyethyl)benzyl]-N-cyclopropyl-5-methyl-6-oxo-5,6-dihydro-3H-spiro[furo[3,4-c]pyridine-1,4′-piperidine]-3′-carboxamide;
(cis-1,3′)-N-[2-Chloro-5-(2-methoxyethyl)benzyl]-N-cyclopropyl-6-methoxy-3H-spiro[furo[3,4-c]pyridine-1,4′-piperidine]-3′-carboxamide;
(cis-1,3′)-N-Benzyl-N-cyclopropyl-5,6-difluoro-3H-spiro[2-benzofuran-1,4′-piperidine]-3′-carboxamide;
(cis-1,3′)-N-Cyclopropyl-N-(2,3-dichlorobenzyl)-5,6-difluoro-3H-spiro[2-benzofuran-1,4′-piperidine]-3′-carboxamide;
(cis-1,3′)-N-Cyclopropyl-N-(2,3-dimethylbenzyl)-5,6-difluoro-3H-spiro[2-benzofuran-1,4′-piperidine]-3′-carboxamide;
(cis-1,3′)-N-Cyclopropyl-5,6-difluoro-N-(4-quinolinylmethyl)-3H-spiro[2-benzofuran-1,4′-piperidine]-3′-carboxamide;
(cis-1,3′)-N-[3-Bromo-5-(3-hydroxypropyl)-4-methylbenzyl]-N-cyclopropyl-5,6-difluoro-3H-spiro[2-benzofuran-1,4′-piperidine]-3′-carboxamide;
(cis-1,3′)-N-[3-Bromo-5-(3-methoxypropyl)-4-methylbenzyl]-N-cyclopropyl-5,6-difluoro-3H-spiro[2-benzofuran-1,4′-piperidine]-3′-carboxamide;
(cis-1,3′)-N-Cyclopropyl-5,6-difluoro-N-(1-naphthylmethyl)-3H-spiro[2-benzofuran-1,4′-piperidine]-3′-carboxamide;
(cis-1,3′)-N-Cyclopropyl-N-(3,4-dichlorobenzyl)-5,6-difluoro-3H-spiro[2-benzofuran-1,4′-piperidine]-3′-carboxamide;
(cis-1,3′)-N-(5-Bromo-2-chlorobenzyl)-N-cyclopropyl-5,6-difluoro-3H-spiro[2-benzofuran-1,4′-piperidine]-3′-carboxamide;
(cis-1,3′)-N-Cyclopropyl-N-[3-(difluoromethoxy)benzyl]-5,6-difluoro-3H-spiro[2-benzofuran-1,4′-piperidine]-3′-carboxamide;
(cis-1,3′)-N-Cyclopropyl-5,6-difluoro-N-[(2-methyl-1-napthyl)methyl]-3H-spiro[2-benzofuran-1,4′-piperidine]-3′-carboxamide;
(cis-1,3′)-N-Cyclopropyl-5,6-difluoro-N-(8-quinolinylmethyl)-3H-spiro[2-benzofuran-1,4′-piperidine]-3′-carboxamide;
(cis-1,3′)-N-Cyclopropyl-N-(2,3-dimethoxybenzyl)-5,6-difluoro-3H-spiro[2-benzofuran-1,4′-piperidine]-3′-carboxamide;
(cis-1,3′)-N-Cyclopropyl-5,6-difluoro-N-[(2-methyl-3-pyridinyl)methyl]-3H-spiro[2-benzofuran-1,4′-piperidine]-3′-carboxamide;
(cis-1,3′)-N-Cyclopropyl-5,6-difluoro-N-[2-methoxy-5-(2-methoxyethyl)benzyl]-3H-spiro[2-benzofuran-1,4′-piperidine]-3′-carboxamide;
(cis-1,3′)-N-Cyclopropyl-5,6-difluoro-N-(1-isoquinolinylmethyl)-3H-spiro[2-benzofuran-1,4′-piperidine]-3′-carboxamide;
(cis-1,3′)-N-[(3-Bromo-1-naphthyl)methyl]-N-cyclopropyl-5,6-difluoro-3H-spiro[2-benzofuran-1,4′-piperidine]-3′-carboxamide;
(cis-1,3′)-N-Cyclopropyl-5,6-difluoro-N-{[3-(3-methoxypropyl)-1-naphthyl]methyl}-3H-spiro[2-benzofuran-1,4′-piperidine]-3′-carboxamide;
(cis-1,3′)-N-Cyclopropyl-5,6-difluoro-N-[(6-methyl-2-pyridinyl)methyl]-3H-spiro[2-benzofuran-1,4′-piperidine]-3′-carboxamide;
(cis-1,3′)-N-Cyclopropyl-5,6-difluoro-N-[(3-methyl-2-pyridinyl)methyl]-3H-spiro[2-benzofuran-1,4′-piperidine]-3′-carboxamide;
(cis-1,3′)-N-(2-Chlorobenzyl)-N-cyclopropyl-5,6-difluoro-3H-spiro[2-benzofuran-1,4′-piperidine]-3′-carboxamide;
(cis-1,3′)-N-(2-Bromobenzyl)-N-cyclopropyl-5,6-difluoro-3H-spiro[2-benzofuran-1,4′-piperidine]-3′-carboxamide;
(cis-1,3′)-N-Cyclopropyl-5,6-difluoro-N-[2-(trifluoromethyl)benzyl]-3H-spiro[2-benzofuran-1,4′-piperidine]-3′-carboxamide;
(cis-1,3′)-N-Cyclopropyl-5,6-difluoro-N-[2-methyl-3-(trifluoromethyl)benzyl]-3H-spiro[2-benzofuran-1,4′-piperidine]-3′-carboxamide;
(cis-1,3′)-N-[(4-Bromo-1-naphthyl)methyl]-N-cyclopropyl-5,6-difluoro-3H-spiro[2-benzofuran-1,4′-piperidine]-3′-carboxamide;
(cis-1,3′)-N-[3-Chloro-5-(trifluoromethyl)benzyl]-N-cyclopropyl-5,6-difluoro-3H-spiro[2-benzofuran-1,4′-piperidine]-3′-carboxamide;
(cis-1,3′)-N-(2-Chloro-4-fluorobenzyl)-N-cyclopropyl-5,6-difluoro-3H-spiro[2-benzofuran-1,4′-piperidine]-3′-carboxamide;
methyl 3-chloro-4-[(cyclopropyl{[(cis-1,3′)-5,6-difluoro-3H-spiro[2-benzofuran-1,4′-piperidin]-3′-yl]carbonyl}amino)methyl]benzoate;
(cis-1,3′)-N-[2-Chloro-3-(trifluoromethyl)benzyl]-N-cyclopropyl-5,6-difluoro-3H-spiro[2-benzofuran-1,4′-piperidine]-3′-carboxamide;
(cis-1,3′)-N-Cyclopropyl-5,6-difluoro-N-[2-methyl-5-(trifluoromethyl)benzyl]-3H-spiro[2-benzofuran-1,4′-piperidine]-3′-carboxamide;
(cis-1,3′)-N-Cyclopropyl-5,6-difluoro-N-[5-(2-methoxyethyl)-2,3-dimethylbenzyl]-3H-spiro[2-benzofuran-1,4′-piperidine]-3′-carboxamide;
(cis-1,3′)-N-Cyclopropyl-5,6-difluoro-N-[5-(3-methoxypropyl)-2,3-dimethylbenzyl]-3H-spiro[2-benzofuran-1,4′-piperidine]-3′-carboxamide;
(cis-1,3′)-N-{3-[2-(Acetylamino)ethyl]benzyl}-N-cyclopropyl-5,6-difluoro-3H-spiro[2-benzofuran-1,4′-piperidine]-3′-carboxamide;
(cis-1,3′)-N-Cyclopropyl-5,6-difluoro-N-{[2-(3-methoxypropyl)-4-quinolinyl]methyl}-3H-spiro[2-benzofuran-1,4′-piperidine]-3′-carboxamide;
(cis-1,3′)-N-{3-[(Acetylamino)methyl]benzyl}-N-cyclopropyl-5,6-difluoro-3H-spiro[2-benzofuran-1,4′-piperidine]-3′-carboxamide;
(cis-1,3′)-N-[(6-Bromo-8-quinolinyl)methyl]-N-cyclopropyl-5,6-difluoro-3H-spiro[2-benzofuran-1,4′-piperidine]-3′-carboxamide;
(cis-1,3′)-N-[(2-Bromo-3-pyridinyl)methyl]-N-cyclopropyl-5,6-difluoro-3H-spiro[2-benzofuran-1,4′-piperidine]-3′-carboxamide;
(cis-1,3′)-N-[(6-Chloro-3-pyridinyl)methyl]-N-cyclopropyl-5,6-difluoro-3H-spiro[2-benzofuran-1,4′-piperidine]-3′-carboxamide;
(cis-1,3′)-N-[(2-Chloro-3,5-bis(3-methoxypropyl)benzyl]-N-cyclopropyl-5,6-difluoro-3H-spiro[2-benzofuran-1,4′-piperidine]-3′-carboxamide;
(cis-1,3′)-N-Cyclopropyl-5,6-difluoro-N-{[6-(3-methoxypropyl)-8-quinolinyl]methyl}-3H-spiro[2-benzofuran-1,4′-piperidine]-3′-carboxamide;
(cis-1,3′)-N-[5-Bromo-2-methyl-3-(trifluoromethyl)benzyl]-N-cyclopropyl-5,6-difluoro-3H-spiro[2-benzofuran-1,4′-piperidine]-3′-carboxamide;
(cis-1,3′)-N-cyclopropyl-5,6-difluoro-N-[(4-fluoro-1H-indol-3-yl)methyl]-3H-spiro[2-benzofuran-1,4′-piperidine]-3′-carboxamide;
(cis-1,3′)-N-Cyclopropyl-5,6-difluoro-N-(1H-pyrrolo[2,3-b]pyridine-3-ylmethyl)-3H-spiro[2-benzofuran-1,4′-piperidine]-3′-carboxamide;
(cis-1,3′)-N-Cyclopropyl-5,6-difluoro-N-[(4-fluoro-1-naphthyl)methyl]-3H-spiro[2-benzofuran-1,4′-piperidine]-3′-carboxamide;
(cis-1,3′)-N-[2-Chloro-3-(trifluoromethyl)benzyl]-N-cyclopropyl-5,6-difluoro-3-oxo-3H-spiro[2-benzofuran-1,4′-piperidine]-3′-carboxamide;
(cis-1,3′)-N-Cyclopropyl-5,6-difluoro-N-[2-methyl-3-(trifluoromethyl)benzyl]-3-oxo-3H-spiro[2-benzofuran-1,4′-piperidine]-3′-carboxamide; or
(cis-1,3′)-N-Cyclopropyl-5,6-difluoro-3-oxo-N-[2-(trifluoromethyl)benzyl]-3H-spiro[2-benzofuran-1,4′-piperidine]-3′-carboxamide.

14. A method of inhibiting renin activity, said method comprising the step of administering a compound according to claim 9 in an amount sufficient to provide an effective amount for renin inhibition in a patient.

15. A pharmaceutical composition comprising a compound of claim 9 or a pharmaceutically acceptable salt thereof, and one or more pharmaceutical excipients.

16. A method of treating hypertension, congestive heart failure, cardiac hypertrophy, cardiac fibrosis, postinfarction cardiomyopathy, nephropathy, vasculopathy, neuropathy, restenosis following angioplasty, raised intra-ocular pressure, glaucoma, abnormal vascular growth, hyperaldosteronism or anxiety states, comprising the step of administering a therapeutically effective amount of a compound according to claim 9 or a pharmaceutically acceptable salt thereof to a patient in need of such treatment.
